(19) United States
(12) Patent Application Publication
(10) Pub. No.: US 2008/0148432 A1
(43) Pub. Date: Jun. 19, 2008

(54) Transgenic plants with enhanced agronomic traits

(76) Inventor: Mark Scott Abad, Webster Grove, MO (US)

Correspondence Address: MONSANTO COMPANY, 800 N. LINDBERGH BLVD., ATTENTION: GAIL P. WUELLNER, IP PARALEGAL, (E2NA), ST. LOUIS, MO, 63167, US

(21) Appl. No.: 11/374,300

(22) Filed: Dec. 21, 2005

(51) Int. Cl. A01H 5/00; C12N 15/82; C12N 5/04

(52) U.S. Cl. 800/279; 800/281; 435/419; 435/468; 800/320.1

(57) ABSTRACT

This invention provides transgenic plant cells with recombinant DNA for expression of proteins that are useful for imparting enhanced agronomic trait(s) to transgenic crop plants. This invention also provides transgenic plants and progeny seed comprising the transgenic plant cells where the plants are selected for having an enhanced trait selected from the group of traits consisting of enhanced water use efficiency, enhanced cold tolerance, increased yield, enhanced nitrogen use efficiency, enhanced seed protein and enhanced seed oil. Also disclosed are methods for manufacturing transgenic seed and plants with enhanced traits.

### CROSS REFERENCE TO RELATED APPLICATIONS

This application claims benefit under 35USC § 119(e) of U.S. provisional application Ser. No. 60/638,099, filed Dec. 21, 2004, herein incorporated by reference.

### INCORPORATION OF SEQUENCE LISTING

Two copies of the sequence listing (Copy 1 and Copy 2) and a computer readable form (CRF) of the sequence listing, all on CD-Rs, each containing the text file named 38-21(53720)D_seqListing.txt, which is 192,434,176 bytes (measured in MS-WINDOWS) and was created on Dec. 21, 2005, are herein incorporated by reference.

### INCORPORATION OF COMPUTER PROGRAM LISTING

Computer Program Listing folders hmmer-2.3.2 and 347pfamDir are contained on a compact disc and is hereby incorporated herein by reference in their entirety. Folder hmmer-2.3.2 contains the source code and other associated file for implementing the HMMer software for Pfam analysis. Folder 347pfamDir contains 347 Pfam Hidden Markov Models. Both folders were created on the disk on Dec. 21, 2005, having a total size of 53,825,536 bytes (measured in MS-WINDOWS).

### INCORPORATION OF TABLE

Two copies of Tables 2 on CD-Rs, each containing the file named 38-21(53720)D_table2.txt, which is 24,334,336 bytes (measured in MS-WINDOWS), was created on Dec. 21, 2005, and comprises 132 pages when viewed in MS Word, are herein incorporated by reference.

### FIELD OF THE INVENTION

Disclosed herein are inventions in the field of plant genetics and developmental biology. More specifically, the present inventions provide plant cells with recombinant DNA for providing an enhanced trait in a transgenic plant, plants comprising such cells, seed and pollen derived from such plants, methods of making and using such cells, plants, seeds and pollen.

### BACKGROUND OF THE INVENTION

Transgenic plants with improved agronomic traits such as yield, environmental stress tolerance, pest resistance, herbicide tolerance, improved seed compositions, and the like are desired by both farmers and consumers. Although considerable efforts in plant breeding have provided significant gains in desired traits, the ability to introduce specific DNA into plant genomes provides further opportunities for generation of plants with improved and/or unique traits. Merely introducing recombinant DNA into a plant genome doesn't always produce a transgenic plant with an enhanced agronomic trait. Methods to select individual transgenic events from a population are required to identify those transgenic events that are characterized by the enhanced agronomic trait.

### SUMMARY OF THE INVENTION

This invention employs recombinant DNA for expression of proteins that are useful for imparting enhanced agronomic traits to the transgenic plants. Recombinant DNA in this invention is provided in a construct comprising a promoter that is functional in plant cells and that is operably linked to DNA that encodes a protein having at least one amino acid domain in a sequence that exceeds the Pfam gathering cutoff for amino acid sequence alignment with a protein domain family identified by a Pfam name in the group of Pfam domain names as identified in Table 12. In more specific embodiments of the invention the protein expressed in plant cells has an amino acid sequence with at least 90% identity to a consensus amino acid sequence in the group of consensus amino acid sequences consisting of the consensus amino acid sequence constructed for SEQ ID NO: 742 and homologs thereof listed in Table 2 through the consensus amino acid sequence constructed for SEQ ID NO: 1482 and homologs thereof listed in Table 2. In even more specific embodiments of the invention the protein expressed in plant cells is a protein selected from the group of proteins identified in Table 1.

Other aspects of the invention are specifically directed to transgenic plant cells comprising the recombinant DNA of the invention, transgenic plants comprising a plurality of such plant cells, progeny transgenic seed, embryo and transgenic pollen from such plants. Such plant cells are selected from a population of transgenic plants regenerated from plant cells transformed with recombinant DNA and that express the protein by screening transgenic plants in the population for an enhanced trait as compared to control plants that do not have said recombinant DNA, where the enhanced trait is selected from group of enhanced traits consisting of enhanced water use efficiency, enhanced cold tolerance, increased yield, enhanced nitrogen use efficiency, enhanced seed protein and enhanced seed oil.

In yet another aspect of the invention the plant cells, plants, seeds, embryo and pollen further comprise DNA expressing a protein that provides tolerance from exposure to an herbicide applied at levels that are lethal to a wild type of said plant cell. Such tolerance is especially useful not only as a advantageous trait in such plants but is also useful in a selection step in the methods of the invention. In aspects of the invention the agent of such herbicide is a glyphosate, dicamba, or glufosinate compound.

Yet other aspects of the invention provide transgenic plants which are homozygous for the recombinant DNA and transgenic seed of the invention from corn, soybean, cotton, canola, alfalfa, wheat or rice plants. In other important embodiments for practice of various aspects of the invention in Argentina the recombinant DNA is provided in plant cells derived from corn lines that that are and maintain resistance to the Mal de Rio Cuarto virus or the Puccina sorghi fungus or both.

This invention also provides methods for manufacturing non-natural, transgenic seed that can be used to produce a crop of transgenic plants with an enhanced trait resulting from expression of stably-integrated, recombinant DNA for expressing a protein having at least one domain of amino acids in a sequence that exceeds the Pfam gathering cutoff for amino acid sequence alignment with a protein domain family identified by a Pfam name in the group of Pfam names identified in Table 12. More specifically the method comprises (a) screening a population of plants for an enhanced trait and a recombinant DNA, where individual plants in the population can exhibit the trait at a level less than, essentially the same as or greater than the level that the trait is exhibited in control plants which do not express the recombinant DNA, (b) selecting from the population one or more plants that exhibit the trait at a level greater than the level that said trait is exhibited in control plants, (c) verifying that the recombinant DNA is stably integrated in said selected plants, (d) analyzing tissue of a selected plant to determine the production of a protein having the function of a protein encoded by nucleotides in a sequence of one of SEQ ID NO: 1-741; and (e) collecting seed from a selected plant. In one aspect of the invention the plants in the population further comprise DNA expressing a protein that provides tolerance to exposure to an herbicide applied at levels that are lethal to wild type plant cells and the selecting is effected by treating the population with the herbicide, e.g. a glyphosate, dicamba, or glufosinate compound. In another aspect of the invention the plants are selected by identifying plants with the enhanced trait. The methods are especially useful for manufacturing corn, soybean, cotton, alfalfa, wheat or rice seed.

Another aspect of the invention provides a method of producing hybrid corn seed comprising acquiring hybrid corn seed from a herbicide tolerant corn plant which also has stably-integrated, recombinant DNA comprising a promoter that is (a) functional in plant cells and (b) is operably linked to DNA that encodes a protein having at least one domain of amino acids in a sequence that exceeds the Pfam gathering cutoff for amino acid sequence alignment with a protein domain family identified by a Pfam name in the group of Pfam names identified in Table 12. The methods further comprise producing corn plants from said hybrid corn seed, wherein a fraction of the plants produced from said hybrid corn seed is homozygous for said recombinant DNA, a fraction of the plants produced from said hybrid corn seed is hemizygous for said recombinant DNA, and a fraction of the plants produced from said hybrid corn seed has none of said recombinant DNA; selecting corn plants which are homozygous and hemizygous for said recombinant DNA by treating with an herbicide; collecting seed from herbicide-treated-surviving corn plants and planting said seed to produce further progeny corn plants; repeating the selecting and collecting steps at least once to produce an inbred corn line; and crossing the inbred corn line with a second corn line to produce hybrid seed.

Another aspect of the invention provides a method of selecting a plant comprising plant cells of the invention by using an immunoreactive antibody to detect the presence of protein expressed by recombinant DNA in seed or plant tissue. Yet another aspect of the invention provides anti-counterfeit milled seed having, as an indication of origin, a plant cells of this invention.

Still other aspects of this invention relate to transgenic plants with enhanced water use efficiency or enhanced nitrogen use efficiency. For instance, this invention provides methods of growing a corn, cotton or soybean crop without irrigation water comprising planting seed having plant cells of the invention which are selected for enhanced water use efficiency. Alternatively methods comprise applying reduced irrigation water, e.g. providing up to 300 millimeters of ground water during the production of a corn crop. This invention also provides methods of growing a corn, cotton or soybean crop without added nitrogen fertilizer comprising planting seed having plant cells of the invention which are selected for enhanced nitrogen use efficiency.

### DETAILED DESCRIPTION OF THE INVENTION

As used herein a “plant cell” means a plant cell that is transformed with stably-integrated, non-natural, recombinant DNA, e.g. by Agrobacterium-mediated transformation or by baombardment using microparticles coated with recombinant DNA or other means. A plant cell of this invention can be an originally-transformed plant cell that exists as a microorganism or as a progeny plant cell that is regenerated into differentiated tissue, e.g. into a transgenic plant with stably-integrated, non-natural recombinant DNA, or seed or pollen derived from a progeny transgenic plant.

As used herein a “transgenic plant” means a plant whose genome has been altered by the stable integration of recombinant DNA. A transgenic plant includes a plant regenerated from an originally-transformed plant cell and progeny transgenic plants from later generations or crosses of a transformed plant.

As used herein “recombinant DNA” means DNA which has been a genetically engineered and constructed outside of a cell including DNA containing naturally occurring DNA or cDNA or synthetic DNA.

As used herein “consensus sequence” means an artificial sequence of amino acids in a conserved region of an alignment of amino acid sequences of homologous proteins, e.g. as determined by a CLUSTALW alignment of amino acid sequence of homolog proteins.

As used herein “homolog” means a protein in a group of proteins that perform the same biological function, e.g. proteins that belong to the same Pfam protein family and that provide a common enhanced trait in transgenic plants of this invention. Homologs are expressed by homologous genes. Homologous genes include naturally occurring alleles and artificially-created variants. Degeneracy of the genetic code provides the possibility to substitute at least one base of the protein encoding sequence of a gene with a different base without causing the amino acid sequence of the polypeptide produced from the gene to be changed. Hence, a polynucleotide useful in the present invention may have any base sequence that has been changed from SEQ ID NO: 1 through SEQ ID NO: 741 substitution in accordance with degeneracy of the genetic code. Homologs are proteins that, when optimally aligned, have at least 60% identity, more preferably about 70% or higher, more preferably at least 80% and even more preferably at least 90% identity over the full length of a protein identified as being associated with imparting an enhanced trait when expressed in plant cells. Homologs include proteins with an amino acid sequence that has at least 90% identity to a consensus amino acid sequence of proteins and homologs disclosed herein.

Homologs are be identified by comparison of amino acid sequence, e.g. manually or by use of a computer-based tool using known homology-based search algorithms such as those commonly known and referred to as BLAST, FASTA, and Smith-Waterman. A local sequence alignment program, e.g. BLAST, can be used to search a database of sequences to find similar sequences, and the summary Expectation value (E-value) used to measure the sequence base similarity. As a protein hit with the best E-value for a particular organism may not necessarily be an ortholog or the only ortholog, a reciprocal query is used in the present invention to filter hit sequences with significant E-values for ortholog identification. The reciprocal query entails search of the significant hits against a database of amino acid sequences from the base organism that are similar to the sequence of the query protein. A hit is a likely ortholog, when the reciprocal query's best hit is the query protein itself or a protein encoded by a duplicated gene after speciation. A further aspect of the invention comprises functional homolog proteins that differ in one or more amino acids from those of disclosed protein as the result of conservative amino acid substitutions, for example substitutions are among: acidic (negatively charged) amino acids such as aspartic acid and glutamic acid; basic (positively charged) amino acids such as arginine, histidine, and lysine; neutral polar amino acids such as glycine, serine, threonine, cysteine, tyrosine, asparagine, and glutamine; neutral nonpolar (hydrophobic) amino acids such as alanine, leucine, isoleucine, valine, proline, phenylalanine, tryptophan, and methionine; amino acids having aliphatic side chains such as glycine, alanine, valine, leucine, and isoleucine; amino acids having aliphatic-hydroxyl side chains such as serine and threonine; amino acids having amide-containing side chains such as asparagine and glutamine; amino acids having aromatic side chains such as phenylalanine, tyrosine, and tryptophan; amino acids having basic side chains such as lysine, arginine, and histidine; amino acids having sulfur-containing side chains such as cysteine and methionine; naturally conservative amino acids such as valine-leucine, valine-isoleucine, phenylalanine-tyrosine, lysine-arginine, alanine-valine, aspartic acid-glutamic acid, and asparagine-glutamine. A further aspect of the homologs encoded by DNA useful in the transgenic plants of the invention are those proteins that differ from a disclosed protein as the result of deletion or insertion of one or more amino acids in a native sequence.

As used herein, “percent identity” means the extent to which two optimally aligned DNA or protein segments are invariant throughout a window of alignment of components, for example nucleotide sequence or amino acid sequence. An “identity fraction” for aligned segments of a test sequence and a reference sequence is the number of identical components that are shared by sequences of the two aligned segments divided by the total number of sequence components in the reference segment over a window of alignment which is the smaller of the full test sequence or the full reference sequence. “Percent identity” (“% identity”) is the identity fraction times 100.

As used herein “Pfam” refers to a large collection of multiple sequence alignments and hidden Markov models covering many common protein families, e.g. Pfam version 18.0 (August 2005) contains alignments and models for 7973 protein families and is based on the Swissprot 47.0 and SP-TrEMBL 30.0 protein sequence databases. See S. R. Eddy, “Profile Hidden Markov Models”, Bioinformatics 14:755-763, 1998. Pfam is currently maintained and updated by a Pfam Consortium. The alignments represent some evolutionary conserved structure that has implications for the protein's function. Profile hidden Markov models (profile HMMs) built from the Pfam alignments are useful for automatically recognizing that a new protein belongs to an existing protein family even if the homology by alignment appears to be low. Once one DNA is identified as encoding a protein which imparts an enhanced trait when expressed in transgenic plants, other DNA encoding proteins in the same protein family are identified by querying the amino acid sequence of protein encoded by candidate DNA against the Hidden Markov Model which characterizes the Pfam domain using HMMER software, a current version of which is provided in the appended computer listing. Candidate proteins meeting the gathering cutoff for the alignment of a particular Pfam are in the protein family and have cognate DNA that is useful in constructing recombinant DNA for the use in the plant cells of this invention. Hidden Markov Model databases for use with HMMER software in identifying DNA expressing protein in a common Pfam for recombinant DNA in the plant cells of this invention are also included in the appended computer listing. The HMMER software and Pfam databases are version 18.0 and were used to identify known domains in the proteins corresponding to amino acid sequence of SEQ ID NO: 742 through SEQ ID NO: 1482. All DNA encoding proteins that have scores higher than the gathering cutoff disclosed in Table 12 by Pfam analysis disclosed herein can be used in recombinant DNA of the plant cells of this invention, e.g. for selecting transgenic plants having enhanced agronomic traits. The relevant Pfams for use in this invention, as more specifically disclosed below, are bZIP_{—}1, bZIP_{—}2, Meth_synt_{—}1, Homeobox, Succ_DH_flav_C, RWP-RK, Meth_synt_{—}2, CTP_synth_N, WD40, Sigma70_r2, Sigma70_r3, Fer4, Sigma70_r4, Sigma70_r1_{—}2, CMAS, Sugar_tr, Rubrerythrin, Pro_dh, Ldh_{—}1_C, START, HATPase_c, Cpn10, Glycos_transf_{—}1, Glycos_transf_{—}2, Pkinase, KH_{—}1, cobW, Ldh_{—}1_N, DUF393, SecY, PCI, SRF-TF, IF4E, Lectin_legA, MatE, Dehydrin, Lectin_legB, Ank, 2-Hacid_dh_C, Tic22, Chal_sti_synt_C, AA_kinase, ELFV_dehydrog_N, HLH, Ribonuclease_T2, HEM4, AT_hook, Peptidase_A22B, tRNA-synt_{—}2b, Suc_Fer-like, Glyco_transf_{—}20, MFS_{—}1, HMA, Ketoacyl-synt_C, Steroid_dh, Hydrolase, Peptidase_C1, Ion_trans, Aa_trans, peroxidase, GAF, Cu-oxidase, ABC1, PMSR, B12D, Chromo, Lipase_GDSL, Ran_BP1, DUF125, Lig_chan, GAT, Tub, NPH3, BAH, GFO_IDH_MocA, DUF6, Orn_DAP_Arg_deC, F-box, 3_{—}5_exonuc, NUDIX, Cyclin_C, Trehalase_Ca-bi, Acyltransferase, MtN3_slv, zf-B_box, PUA, AMPKBI, Peptidase_M20, Transaldolase, ketoacyl-synt, Cyclin_N, HisKA, Ribosomal_L7Ae, Methyltransf_{—}11, Methyltransf_{—}12, Hexapep, Ribosomal_S2, Jacalin, ERp29, MFMR, Usp, DUF641, Pyr_redox_dim, Auxin_resp, Inhibitor_{—}129, Transferase, cNMP_binding, BURP, Epimerase, Ribosomal_L39, Metallothio_{—}2, Pyr_redox_{—}2, WRKY, GSHPx, Kelch_{—}1, Kelch_{—}2, Aminotran_{—}1_{—}2, ABC_tran, UDPGT, Cystatin, YL1, AMP-binding, NTP_transferase, HALZ, Kunitz_legume, HSP20, DUF581, FGGY_N, Aminotran_{—}3, PHD, B56, Aminotran_{—}5, PSI_PsaF, malic, zf-C2H2, HEAT, UPF0057, Asn_synthase, K-box, HAMP, PTR2, SapB_{—}1, Ammonium_transp, SapB_{—}2, GATase, Pyr_redox, Cu-oxidase_{—}2, Cu-oxidase_{—}3, Cyclotide, Asp, M20_dimer, PA, Thiolase_C, FHA, YjeF_N, Citrate_synt, GTP_EFTU_D2, GTP_EFTU_D3, PK, GATA, Thiolase_N, Glycogen_syn, WHEP-TRS, B3, EF1_GNE, FAD_binding_{—}3, ComA, Remorin_C, FAD_binding_{—}7, RmlD_sub_bind, CBS, ELFV_dehydrog, YL1_C, zf-Dof, Ribosomal_S11, ArfGap, GRAS, Metallophos, Annexin, Ras, NAC, Acetyltransf_{—}1, Ribosomal_S17, NAF, DUF246, GST_C, CN_hydrolase, Na_Ca_ex, DUF1423, Ubie_methyltran, p450, PP2C, NAM, Histone, GST_N, Tubulin, 2-Hacid_dh, Ribosomal_L19e, CCT, Malic_M, PK_C, VHS, IPK, HSF_DNA-bind, Tubulin_C, Sina, JmjC, CH, Catalase, DUF250, HMG_box, PfkB, Yippee, DSPc, Pkinase_C, UbiA, Ribosomal_S27, ADH_zinc_N, Zip, Globin, JmjN, Cys_Met_Meta_PP, H10933_like, GH3, Bromodomain, ERO1, DAO, DUF760, Methyltransf_{—}2, Gp_dh_C, HGTP_anticodon, Methyltransf 3, Aldo_ket_red, Thioredoxin, NmrA, SelR, LEA_{—}5, Orn_Arg_deC_N, Polysacc_synt_{—}2, Gp_dh_N, NifU_N, GFO_IDH_MocA_C, Gamma-thionin, FBA_{—}1, H_PPase, ADH_N, Heme_oxygenase, AUX_IAA, NAD_binding_{—}4, Auxin_inducible, LIM, Response_reg, Dirigent, E2F_TDP, Di19, Alpha_adaptinC2, efhand, ICL, Rieske, GTP_EFTU, ARID, adh_short, Transket_pyr, AA_permease, TPP_enzyme_C, NDK, RRM_{—}1, Trypsin, Pro_CA, Hexokinase_{—}1, CBFD_NFYB_HMF, Glyco_hydro_{—}38C, TPP_enzyme_M, TPP_enzyme_N, Hexokinase_{—}2, 3Beta_HSD, DUF788, Wzy_C, E1_dh, Glycolytic, RuBisCO_small, ZF-HD_dimer, DUF1530, PARP, Pyridoxal_deC, IlvC, Ribosomal_L1, Alpha-amylase, EB1, CorA, Sucrose_synth, PGAM, IlvN, MAP1_LC3, DNA_photolyase, PAD_porph, Abhydrolase_{—}1, Glyco_hydro_{—}16, NTF2, CobW_C, GATase_{—}2, Cation_efflux, Gln-synt_C, VQ, DUF296, W2, SAM_{—}1, SAM_{—}2, Gln-synt_N, Transketolase_C, PEPcase, GRIM-19, Pkinase_Tyr, DnaJ, MIP, PRA1, Trehalose_PPase, Transketolase_N, LRR_{—}2, KA1, Mpv17_PMP22, Reticulon, Trp_syntA, YTH, Aldedh, zf-C3HC4, GIDA, Trp_Tyr_perm, UBA, PB1, PAS, Carb_kinase, zf-LSD1, CAF1, Xan_ur_permease, Hist_deacetyl, Cpn60_TCP1, XET_C, Ribosomal_L10e, Trehalase, ubiquitin, Glyco_hydro_{—}38, AP2, Myb_DNA-binding, APS_kinase, PBD, FAE_{—}3-kCoA_syn1, the databases for which are included in the appended computer listing.

As used herein “promoter” means regulatory DNA for initializing transcription. A “plant promoter” is a promoter capable of initiating transcription in plant cells whether or not its origin is a plant cell, e.g. is it well known that Agrobacterium promoters are functional in plant cells. Thus, plant promoters include promoter DNA obtained from plants, plant viruses and bacteria such as Agrobacterium and Bradyrhizobium bacteria. Examples of promoters under developmental control include promoters that preferentially initiate transcription in certain tissues, such as leaves, roots, or seeds. Such promoters are referred to as “tissue preferred”. Promoters that initiate transcription only in certain tissues are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions, or certain chemicals, or the presence of light. Tissue specific, tissue preferred, cell type specific, and inducible promoters constitute the class of “non-constitutive” promoters. A “constitutive” promoter is a promoter which is active under most conditions.

As used herein “operably linked” means the association of two or more DNA fragments in a DNA construct so that the function of one, e.g. protein-encoding DNA, is controlled by the other, e.g. a promoter.

As used herein “expressed” means produced, e.g. a protein is expressed in a plant cell when its cognate DNA is transcribed to mRNA that is translated to the protein.

As used herein a “control plant” means a plant that does not contain the recombinant DNA that expressed a protein that impart an enhanced trait. A control plant is to identify and select a transgenic plant that has an enhance trait. A suitable control plant can be a non-transgenic plant of the parental line used to generate a transgenic plant, i.e. devoid of recombinant DNA. A suitable control plant may in some cases be a progeny of a hemizygous transgenic plant line that is does not contain the recombinant DNA, known as a negative segregant.

As used herein an “enhanced trait” means a characteristic of a transgenic plant that includes, but is not limited to, an enhance agronomic trait characterized by enhanced plant morphology, physiology, growth and development, yield, nutritional enhancement, disease or pest resistance, or environmental or chemical tolerance. In more specific aspects of this invention enhanced trait is selected from group of enhanced traits consisting of enhanced water use efficiency, enhanced cold tolerance, increased yield, enhanced nitrogen use efficiency, enhanced seed protein and enhanced seed oil. In an important aspect of the invention the enhanced trait is enhanced yield including increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also affected by efficiency of germination (including germination in stressed conditions), growth rate (including growth rate in stressed conditions), ear number, seed number per ear, seed size, composition of seed (starch, oil, protein) and characteristics of seed fill.

Increased yield of a transgenic plant of the present invention can be measured in a number of ways, including test weight, seed number per plant, seed weight, seed number per unit area (i.e. seeds, or weight of seeds, per acre), bushels per acre, tonnes per acre, tons per acre, kilo per hectare. For example, maize yield may be measured as production of shelled corn kernels per unit of production area, for example in bushels per acre or metric tons per hectare, often reported on a moisture adjusted basis, for example at 15.5 percent moisture. Increased yield may result from improved utilization of key biochemical compounds, such as nitrogen, phosphorous and carbohydrate, or from improved responses to environmental stresses, such as cold, heat, drought, salt, and attack by pests or pathogens. Recombinant DNA used in this invention can also be used to provide plants having improved growth and development, and ultimately increased yield, as the result of modified expression of plant growth regulators or modification of cell cycle or photosynthesis pathways. Also of interest is the generation of transgenic plants that demonstrate enhanced yield with respect to a seed component that may or may not correspond to an increase in overall plant yield. Such properties include enhancements in seed oil, seed molecules such as tocopherol, protein and starch, or oil particular oil components as may be manifest by an alterations in the ratios of seed components.

A subset of the nucleic molecules of this invention includes fragments of the disclosed recombinant DNA consisting of oligonucleotides of at least 15, preferably at least 16 or 17, more preferably at least 18 or 19, and even more preferably at least 20 or more, consecutive nucleotides. Such oligonucleotides are fragments of the larger molecules having a sequence selected from the group consisting of SEQ ID NO: 1 through SEQ ID NO: 741, and find use, for example as probes and primers for detection of the polynucleotides of the present invention.

In some embodiments of the present invention, a dominant negative mutant of a native gene is generated to achieve the desired effect. As used herein, “dominant negative mutant” means a mutant gene whose gene product adversely affects the normal, wild-type gene product within the same cell, usually by dimerizing (combining) with it. In cases of polymeric molecules, such as collagen, dominant negative mutations are often more deleterious than mutations causing the production of no gene product (null mutations or null alleles). For example, SEQ ID NO: 839-842 are constructed to encode agl11 protein with K-box deleted or C-terminal domain deleted. AGL11 or MADS box protein can be considered as having three functional domains. There is an N-terminal DNA-binding domain (the MADS box), a more distal dimerization domain (the K-box) and a C-terminal domain that is usually involved in interactions with other proteins. In plants the region between the MADS box and the K-box has been shown to be important for DNA binding in some proteins and is often referred to as the 1-box (Fan et al., 1997). Several different classes of dominant negative constructs are considered. Deletion or inactivation of the DNA-binding domain can create proteins that are able to dimerize with their native full length counterparts as well as other natural dimerization partners. Likewise, removal of the C-terminal domain can allow dimerization with both the native protein and it's natural dimerization partners. In both cases these types of constructs disable both the target protein and any other protein capable of interacting with the K-box.

In other embodiments of the invention a constitutively active mutant is constructed to achieve the desired effect. For example, SEQ ID NO: 831 and SEQ ID NO: 832 encode only the kinase domain from a calcium-dependent protein kinase (CDPK). CDPK1 has a domain structure similar to other calcium-dependant protein kinases in which the protein kinase domain is separated from four efhand domains by 42 amino acid “spacer” region. Calcium-dependant protein kinases are thought to be activated by a calcium-induced conformational change that results in movement of an autoinhibitory domain away from the protein kinase active site (Yokokura et al., 1995). Thus, constitutively active proteins can be made by over expressing the protein kinase domain alone.

DNA constructs are assembled using methods well known to persons of ordinary skill in the art and typically comprise a promoter operably linked to DNA, the expression of which provides the enhanced agronomic trait. Other construct components may include additional regulatory elements, such as 5′ leasders and introns for enhancing transcription, 3′ untranslated regions (such as polyadenylation signals and sites), DNA for transit or signal peptides.

Numerous promoters that are active in plant cells have been described in the literature. These include promoters present in plant genomes as well as promoters from other sources, including nopaline synthase (NOS) promoter and octopine synthase (OCS) promoters carried on tumor-inducing plasmids of Agrobacterium tumefaciens, caulimovirus promoters such as the cauliflower mosaic virus. For instance, see U.S. Pat. Nos. 5,858,742 and 5,322,938, which disclose versions of the constitutive promoter derived from cauliflower mosaic virus (CaMV35S), U.S. Pat. No. 5,641,876, which discloses a rice actin promoter, U.S. Patent Application Publication 2002/0192813A1, which discloses 5′, 3′ and intron elements useful in the design of effective plant expression vectors, U.S. patent application Ser. No. 09/757,089, which discloses a maize chloroplast aldolase promoter, U.S. patent application Ser. No. 08/706,946, which discloses a rice glutelin promoter, U.S. patent application Ser. No. 09/757,089, which discloses a maize aldolase (FDA) promoter, and U.S. patent application Ser. No. 60/310,370, which discloses a maize nicotianamine synthase promoter, all of which are incorporated herein by reference. These and numerous other promoters that function in plant cells are known to those skilled in the art and available for use in recombinant polynucleotides of the present invention to provide for expression of desired genes in transgenic plant cells.

In other aspects of the invention, preferential expression in plant green tissues is desired. Promoters of interest for such uses include those from genes such as Arabidopsis thaliana ribulose-1,5-bisphosphate carboxylase (Rubisco) small subunit (Fischhoff et al. (1992) Plant Mol Biol. 20:81-93), aldolase and pyruvate orthophosphate dikinase (PPDK) (Taniguchi et al. (2000) Plant Cell Physiol. 41(11):42-48).

Furthermore, the promoters may be altered to contain multiple “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted upstream (5′) or downstream (3′) to the coding sequence. In some instances, these 5′ enhancing elements are introns. Particularly useful as enhancers are the 5′ introns of the rice actin 1 (see U.S. Pat. No. 5,641,876) and rice actin 2 genes, the maize alcohol dehydrogenase gene intron, the maize heat shock protein 70 gene intron (U.S. Pat. No. 5,593,874) and the maize shrunken 1 gene.

In other aspects of the invention, sufficient expression in plant seed tissues is desired to effect improvements in seed composition. Exemplary promoters for use for seed composition modification include promoters from seed genes such as napin (U.S. Pat. No. 5,420,034), maize L3 oleosin (U.S. Pat. No. 6,433,252), zein Z27 (Russell et al. (1997) Transgenic Res. 6(2): 157-166), globulin 1 (Belanger et al (1991) Genetics 129:863-872), glutelin 1 (Russell (1997) supra), and peroxiredoxin antioxidant (Per1) (Stacy et al. (1996) Plant Mol Biol. 31(6): 1205-1216).

Recombinant DNA constructs prepared in accordance with the invention will also generally include a 3′ element that typically contains a polyadenylation signal and site. Well-known 3′ elements include those from Agrobacterium tumefaciens genes such as nos 3′, tml 3′, tmr 3′, tms 3, ocs 3′, tr7 3′, for example disclosed in U.S. Pat. No. 6,090,627, incorporated herein by reference; 3′ elements from plant genes such as wheat (Triticum aesevitum) heat shock protein 17 (Hsp17 3′), a wheat ubiquitin gene, a wheat fructose-1,6-biphosphatase gene, a rice glutelin gene a rice lactate dehydrogenase gene and a rice beta-tubulin gene, all of which are disclosed in U.S. published patent application 2002/0192813 A1, incorporated herein by reference; and the pea (Pisum sativum) ribulose biphosphate carboxylase gene (rbs 3), and 3′ elements from the genes within the host plant.

Constructs and vectors may also include a transit peptide for targeting of a gene target to a plant organelle, particularly to a chloroplast, leucoplast or other plastid organelle. For descriptions of the use of chloroplast transit peptides see U.S. Pat. No. 5,188,642 and U.S. Pat. No. 5,728,925, incorporated herein by reference. For description of the transit peptide region of an Arabidopsis EPSPS gene useful in the present invention, see Klee, H. J. et al (MGG (1987) 210:437-442).

Transgenic plants comprising or derived from plant cells of this invention transformed with recombinant DNA can be further enhanced with stacked traits, e.g. a crop plant having an enhanced trait resulting from expression of DNA disclosed herein in combination with herbicide and/or pest resistance traits. For example, genes of the current invention can be stacked with other traits of agronomic interest, such as a trait providing herbicide resistance, or insect resistance, such as using a gene from Bacillus thuringensis to provide resistance against lepidopteran, coliopteran, homopteran, hemiopteran, and other insects. Herbicides for which transgenic plant tolerance has been demonstrated and the method of the present invention can be applied include, but are not limited to, glyphosate, dicamba, glufosinate, sulfonylurea, bromoxynil and norflurazon herbicides. Polynucleotide molecules encoding proteins involved in herbicide tolerance are well-known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Patent Application publication 2003/0083480 A1 also for imparting glyphosate tolerance; dicamba monooxygenase disclosed in U.S. Patent Application publication 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al, (1993) Plant J 4:833-840 and Misawa et al, (1994) Plant J 6:481-489 for norflurazon tolerance; a polynucleotide molecule encoding acetohydroxyacid synthase (AHAS, aka ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance; polynucleotide molecules disclosed in U.S. Patent Application Publication 2003/010609 A1 for imparting N-amino methyl phosphonic acid tolerance; polynucleotide molecules disclosed in U.S. Pat. No. 6,107,549 for imparting pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and U.S. Patent Application Publication 2002/0112260, all of said U.S. patents and patent application Publications are incorporated herein by reference. Molecules and methods for imparting insect/nematode/virus resistance is disclosed in U.S. Pat. Nos. 5,250,515; 5,880,275; 6,506,599; 5,986,175 and U.S. Patent Application Publication 2003/0150017 A1, all of which are incorporated herein by reference.

In particular embodiments, the inventors contemplate the use of antibodies, either monoclonal or polyclonal which bind to the proteins disclosed herein. Means for preparing and characterizing antibodies are well known in the art (See, e.g., Antibodies: A Laboratory Manual, Cold Spring Harbor Laboratory, 1988; incorporated herein by reference). The methods for generating monoclonal antibodies (mAbs) generally begin along the same lines as those for preparing polyclonal antibodies. Briefly, a polyclonal antibody is prepared by immunizing an animal with an immunogenic composition in accordance with the present invention and collecting antisera from that immunized animal. A wide range of animal species can be used for the production of antisera. Typically the animal used for production of anti-antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include using glutaraldehyde, m-maleimidobencoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified antifungal protein, polypeptide or peptide. The immunizing composition is administered in a manner effective to stimulate antibody producing cells. Rodents such as mice and rats are preferred animals, however, the use of rabbit, sheep, or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

Following immunization, somatic cells with the potential for producing antibodies, specifically B lymphocytes (B cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10 to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render them incapable of growing in certain selective media which support the growth of only the desired fused cells (hybridomas).

Any one of a number of myeloma cells may be used, as are known to those of skill in the art (Goding, 1986, pp. 65-66; Campbell, 1984, pp. 75-83). For example, where the immunized animal is a mouse, one may use P3-X63/Ag8, X63-Ag8.653, NS1/1.Ag 41, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Spend virus have been described (Kohler and Milstein, 1975; 1976), and those using polyethylene glycol (PEG), such as 37% (v/v) PEG, (Gefter et al., 1977). The use of electrically induced fusion methods is also appropriate (Goding, 1986, pp. 71-74).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azasenne blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

### Plant Cell Transformation Methods

Numerous methods for transforming plant cells with recombinant DNA are known in the art and may be used in the present invention. Two commonly used methods for plant transformation are Agrobacterium-mediated transformation and microprojectile bombardment. Microprojectile bombardment methods are illustrated in U.S. Pat. Nos. 5,015,580 (soybean); 5,550,318 (corn); 5,538,880 (corn); 5,914,451 (soybean); 6,160,208 (corn); 6,399,861 (corn) and 6,153,812 (wheat) and Agrobacterium-mediated transformation is described in U.S. Pat. Nos. 5,159,135 (cotton); 5,824,877 (soybean); 5,591,616 (corn); and 6,384,301 (soybean), all of which are incorporated herein by reference. For Agrobacterium tumefaciens based plant transformation system, additional elements present on transformation constructs will include T-DNA left and right border sequences to facilitate incorporation of the recombinant polynucleotide into the plant genome.

In general it is useful to introduce recombinant DNA randomly, i.e. at a non-specific location, in the genome of a target plant line. In special cases it may be useful to target recombinant DNA insertion in order to achieve site-specific integration, for example to replace an existing gene in the genome, to use an existing promoter in the plant genome, or to insert a recombinant polynucleotide at a predetermined site known to be active for gene expression. Several site specific recombination systems exist which are known to function implants include cre-lox as disclosed in U.S. Pat. No. 4,959,317 and FLP-FRT as disclosed in U.S. Pat. No. 5,527,695, both incorporated herein by reference.

Transformation methods of this invention are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Cells capable of proliferating as callus are also recipient cells for genetic transformation. Practical transformation methods and materials for making transgenic plants of this invention, for example various media and recipient target cells, transformation of immature embryo cells and subsequent regeneration of fertile transgenic plants are disclosed in U.S. Pat. Nos. 6,194,636 and 6,232,526, which are incorporated herein by reference.

The seeds of transgenic plants can be harvested from fertile transgenic plants and be used to grow progeny generations of transformed plants of this invention including hybrid plants line for selection of plants having an enhanced trait. In addition to direct transformation of a plant with a recombinant DNA, transgenic plants can be prepared by crossing a first plant having a recombinant DNA with a second plant lacking the DNA. For example, recombinant DNA can be introduced into first plant line that is amenable to transformation to produce a transgenic plant which can be crossed with a second plant line to introgress the recombinant DNA into the second plant line. A transgenic plant with recombinant DNA providing an enhanced trait, e.g. enhanced yield, can be crossed with transgenic plant line having other recombinant DNA that confers another trait, for example herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA, e.g. marker identification by analysis for recombinant DNA or, in the case where a selectable marker is linked to the recombinant, by application of the selecting agent such as a herbicide for use with a herbicide tolerance marker, or by selection for the enhanced trait. Progeny plants carrying DNA for both parental traits can be crossed back into the female parent line multiple times, for example usually 6 to 8 generations, to produce a progeny plant with substantially the same genotype as one original transgenic parental line but for the recombinant DNA of the other transgenic parental line

In the practice of transformation DNA is typically introduced into only a small percentage of target plant cells in any one transformation experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (nptII), hygromycin B (aph IV) and gentamycin (aac3 and aacC4) or resistance to herbicides such as glufosinate (bar or pat) and glyphosate (aroA or EPSPS). Examples of such selectable are illustrated in U.S. Pat. Nos. 5,550,318; 5,633,435; 5,780,708 and 6,118,047, all of which are incorporated herein by reference. Selectable markers which provide an ability to visually identify transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO₂, and 25-250 microeinsteins m⁻² s⁻¹ of light, prior to transfer to a greenhouse or growth chamber for maturation. Plants are regenerated from about 6 weeks to 10 months after a transformant is identified, depending on the initial tissue. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of enhanced agronomic trait.

### Transgenic Plants and Seeds

Transgenic plants derived from the plant cells of this invention are grown to generate transgenic plants having an enhanced trait as compared to a control plant and produce transgenic seed and haploid pollen of this invention. Such plants with enhanced traits are identified by selection of transformed plants or progeny seed for the enhanced trait. For efficiency a selection method is designed to evaluate multiple transgenic plants (events) comprising the recombinant DNA, for example multiple plants from 2 to 20 or more transgenic events. Transgenic plants grown from transgenic seed provided herein demonstrate improved agronomic traits that contribute to increased yield or other trait that provides increased plant value, including, for example, improved seed quality. Of particular interest are plants having enhanced water use efficiency, enhanced cold tolerance, increased yield, enhanced nitrogen use efficiency, enhanced seed protein and enhanced seed oil.

Table 1 provides a list of protein encoding DNA (“genes”) that are useful as recombinant DNA for production of transgenic plants with enhanced agronomic trait, the elements of Table 1 are described by reference to:

“PEP SEQ” which identifies an amino acid sequence from SEQ ID NO: 742 to 1482.
“NUC SEQ” which identifies a DNA sequence from SEQ ID NO: 1 to 741.
“Base Vector” is a reference to the identifying number in Table 4 of base vectors used for construction of the transformation vectors of the recombinant DNA. Construction of plant transformation constructs is illustrated in Example 1.
“PROTEIN NAME” which is a common name for protein encoded by the recombinant DNA.

TABLE 1

| PEP | NUC | | | |
|---|---|---|---|---|
| SEQ | SEQ | | | |
| ID | ID | | Base | |
| NO | NO | GENE ID | Vector | PPROTEIN NAME |
| 742 | 1 | PHE0001089_1179 | 1 | Arabidopsis NAC domain transcription factor |
| 743 | 2 | PHE0001133_1223 | 1 | yeast aspartate aminotransferase (AAT2) |
| 744 | 3 | PHE0001134_1224 | 1 | yeast aspartate aminotransferase (AAT1)- |
| | | | | CAA97550 |
| 745 | 4 | PHE0001135_1225 | 1 | E. coli aspC-1651442 |
| 746 | 5 | PHE0001181_1271 | 1 | rice IAA1-like 1-AJ251791 |
| 747 | 6 | PHE0001227_1317 | 1 | yeast CTT1-NP_011602 |
| 748 | 7 | PHE0001228_1318 | 1 | yeast STE20-AAB69747 |
| 749 | 8 | PHE0001247_1338 | 1 | Arabidopsis cyclin D3-like-CGPG 710 |
| 750 | 9 | PHE0001252_1343 | 1 | Arabidopsis arabinogalactan-protein 1 |
| 751 | 10 | PHE0001267_1358 | 1 | Arabidopsis hypothetical protein |
| 752 | 11 | PHE0001275_1336 | 1 | Arabidopsis CIP8 |
| 753 | 12 | PHE0001287_1377 | 1 | Arabidopsis hypothetical protein |
| 754 | 13 | PHE0001382_1474 | 1 | rice Short-Root SHR-like transcriptional |
| | | | | factor 1 sequence- |
| 755 | 14 | PHE0001531_1622 | 1 | Bacillus subtilis GDH |
| 756 | 15 | PHE0001532_1623 | 1 | Saccharomyces cerevisiae GDH-1431821 |
| 757 | 16 | PHE0001535_1626 | 1 | Pseudomonas fluorescens GDH |
| 758 | 17 | PHE0001536_1627 | 1 | Bacillus halodurans GDH |
| 759 | 18 | PHE0001538_1629 | 1 | Synechocystis sp. 6803 GDH |
| 760 | 19 | PHE0001593_1699 | 5 | yeast transketolase |
| 761 | 20 | PHE0001593_1700 | 1 | yeast transketolase |
| 762 | 21 | PHE0002070_2180 | 1 | rice G664-like 2 |
| 763 | 22 | PHE0002128_2236 | 1 | E. coli phosphoenolpyruvate carboxylase |
| 764 | 23 | PHE0002258_69 | 2 | corn nphI-S851D |
| 765 | 24 | PHE0002259_68 | 2 | corn nphI-S849D |
| 766 | 25 | PHE0002260_70 | 2 | corn nphI-S849D-S851D |
| 767 | 26 | PHE0002645_2764 | 1 | Arabidopsis GH3 protein 3 |
| 768 | 27 | PHE0002976_3126 | 1 | Yeast Serine/Threonine Protein Kinase |
| 769 | 28 | PHE0002999_3149 | 1 | Yeast Stereospecific (2R,3R)-2,3-butanediol |
| | | | | dehydrogenase with similarity to |
| | | | | alcohol/sorbitol dehydrogenases |
| 770 | 29 | PHE0003000_3150 | 1 | Yeast Car2p: Ornithine aminotransferase |
| 771 | 30 | PHE0003001_3151 | 1 | Yeast alpha-Mannosidase |
| 772 | 31 | PHE0003002_3152 | 1 | Yeast Putative indole-3-pyruvate |
| | | | | decarboxylase |
| 773 | 32 | PHE0003004_3154 | 1 | Yeast Citrate synthase |
| 774 | 33 | PHE0003006_3156 | 1 | Yeast Galactose-induced protein with strong |
| | | | | similarity to crystallin protein of vertebrate |
| | | | | eye lens |
| 775 | 34 | PHE0003007_3157 | 1 | Yeast Phosphoglycerate mutase |
| 776 | 35 | PHE0003008_3158 | 1 | Yeast Heat shock protein of 26 kDa, |
| | | | | expressed during entry to stationary phase and |
| | | | | induced by osmostress |
| 777 | 36 | PHE0003012_3162 | 1 | Yeast Neutral trehalase (alpha, alpha- |
| | | | | trehalase), catalyzes the conversion of |
| | | | | intracellular trehalose to glucose |
| 778 | 37 | PHE0003014_3164 | 1 | YEAST P16547 MITOCHONDRIAL |
| | | | | OUTER MEMBRANE 45 KD PROTEIN |
| 779 | 38 | PHE0003022_3172 | 1 | Yeast Protein of unknown function, has |
| | | | | strong similarity to Tal1p (Transaldolase |
| | | | | PFAM domain) |
| 780 | 39 | PHE0003031_3181 | 1 | Yeast Long-chain fatty acid CoA ligase (fatty |
| | | | | acid activator 1) |
| 781 | 40 | PHE0003035_3185 | 1 | Yeast Glucokinase, specific for aldohexoses |
| 782 | 41 | PHE0003037_3187 | 1 | Yeast Aldose reductase with NADPH |
| | | | | specificity; induced by osmotic stress |
| 783 | 42 | PHE0003044_3194 | 1 | Yeast Calcium/calmodulin-dependent |
| | | | | serine/threonine protein kinase (CaM kinase) |
| | | | | type II |
| 784 | 43 | PHE0003056_3206 | 1 | yeast CBK1 |
| 785 | 44 | PHE0003166_3368 | 6 | Agrobacterium tumefaciens str. C58 sucrose |
| | | | | phosphorylase |
| 786 | 45 | PHE0003191_3390 | 1 | rice G1225-like 1 |
| 787 | 46 | PHE0003266_3485 | 1 | Arabidopsis unknown protein |
| 788 | 47 | PHE0003295_3515 | 6 | rice unknown protein |
| 789 | 48 | PHE0003358_3581 | 16 | rice dwarf4-like |
| 790 | 49 | PHE0003416_3656 | 16 | Arabidopsis homogentisate |
| | | | | phytylprenyltransferase |
| 791 | 50 | PHE0003457_3688 | 1 | rice G1660 like 1 |
| 792 | 51 | PHE0003502_3748 | 4 | rice G2239 like1 |
| 793 | 52 | PHE0003506_3752 | 1 | rice G2317 like2 |
| 794 | 53 | PHE0003522_3768 | 1 | rice G2536 like1 |
| 795 | 54 | PHE0003592_3838 | 1 | rice G864 like1 |
| 796 | 55 | PHE0003595_3841 | 1 | rice G46 like1 |
| 797 | 56 | PHE0003633_3891 | 1 | rice G2930 like 1 |
| 798 | 57 | PHE0003635_3893 | 1 | rice G2969 like2 |
| 799 | 58 | PHE0003663_3921 | 1 | rice G1108 like2 |
| 800 | 59 | PHE0003670_3928 | 1 | rice G1792 like3 |
| 801 | 60 | PHE0003672_3930 | 4 | rice G1013 like2 |
| 802 | 61 | PHE0003675_3933 | 1 | rice G1493 like3 |
| 803 | 62 | PHE0003945_4522 | 4 | Arabidopsis CGPG2571 |
| 804 | 63 | PHE0001066_1156 | 1 | yeast SIP1-AAB64887 |
| 805 | 64 | PHE0001471_1563 | 1 | rice NH4-uniport AMT1-like sequence- |
| 806 | 65 | PHE0001530_1621 | 1 | Emericella nidulans GDH |
| 807 | 66 | PHE0002940_3090 | 1 | Yeast Proline oxidase (proline |
| | | | | dehydrogenase) |
| 808 | 67 | PHE0002957_3107 | 1 | Yeast MIP (member of major intrinsic protein |
| | | | | family of transmembrane channels) |
| 809 | 68 | PHE0002961_3111 | 1 | Yeast protein with 3 RRM domains (RNA |
| | | | | recongnition motifs) |
| 810 | 69 | PHE0002962_3112 | 1 | Yeast Putative Dual Specificity Phosphatase |
| 811 | 70 | PHE0003003_3153 | 1 | Yeast Aromatic amino acid aminotransferase |
| | | | | II |
| 812 | 71 | PHE0003024_3174 | 1 | Yeast protein of unknown function |
| 813 | 72 | PHE0003042_3192 | 1 | Yeast Possible 6-phosphogluconolactonase |
| 814 | 73 | PHE0003236_3451 | 1 | Arabidopsis unknown protein |
| 815 | 74 | PHE0003280_3499 | 1 | Arabidopsis DUF6 |
| 816 | 75 | PHE0000035_61 | 1 | corn lip19 |
| 817 | 76 | PHE0000036_62 | 1 | corn EF1-alpha |
| 818 | 77 | PHE0000130_220 | 2 | b-glucosidase-aggregating factor |
| 819 | 78 | PHE0000134_224 | 2 | LEA protein EMB5 |
| 820 | 79 | PHE0000135_225 | 2 | PP2C |
| 821 | 80 | PHE0000136_226 | 2 | histone deacetylase |
| 822 | 81 | PHE0000139_229 | 2 | ascorbate peroxidase |
| 823 | 82 | PHE0000141_231 | 1 | cp drought-induced stress protein |
| 824 | 83 | PHE0000142_232 | 2 | rab7a |
| 825 | 84 | PHE0000146_236 | 1 | rab11d |
| 826 | 85 | PHE0000148_238 | 2 | protein kinase |
| 827 | 86 | PHE0000149_239 | 2 | EREbp-like AP2 domain TF |
| 828 | 87 | PHE0000150_240 | 2 | protein kinase |
| 829 | 88 | PHE0000189_282 | 1 | soy HSP20 (HS11) |
| 830 | 89 | PHE0000200_293 | 1 | protein phosphatase 2C-like |
| 831 | 90 | PHE0000211_304 | 2 | CDPK kinase domain |
| 832 | 91 | PHE0000213_306 | 2 | CDPK kinase domain |
| 833 | 92 | PHE0000368_459 | 1 | rice mlip19 |
| 834 | 93 | PHE0000369_460 | 1 | OSK5 |
| 835 | 94 | PHE0000370_461 | 1 | abscisic acid-inducible protein kinase |
| 836 | 95 | PHE0000780_853 | 1 | Arabidopsis agl11 |
| 837 | 96 | PHE0000782_855 | 4 | corn agl11-like 1 |
| 838 | 97 | PHE0000783_856 | 1 | rice MADS3-L37528 |
| 839 | 98 | PHE0000785_858 | 1 | Arabidopsis agl11 delta C-terminus |
| 840 | 99 | PHE0000791_864 | 1 | soy agl11-like 1 delta C-terminus |
| 841 | 100 | PHE0000792_865 | 1 | soy agl11-like 1 delta K-box |
| 842 | 101 | PHE0000794_867 | 1 | rice MADS3 delta C-terminus-L37528 |
| 843 | 102 | PHE0000821_896 | 1 | corn duf6 10 |
| 844 | 103 | PHE0001051_1141 | 1 | corn zinc finger protein |
| 845 | 104 | PHE0001053_4292 | 11 | corn NAM-like protein |
| 846 | 105 | PHE0001054_1144 | 1 | corn QKI-like RNA-binding protein |
| 847 | 106 | PHE0001055_1145 | 1 | corn putative kinase regulatory subunit |
| 848 | 107 | PHE0001056_1146 | 1 | corn unknown protein |
| 849 | 108 | PHE0001057_1147 | 1 | corn thionin-like protease inhibitor |
| 850 | 109 | PHE0001064_1154 | 1 | yeast GAL83-Q04739 |
| 851 | 110 | PHE0001071_1161 | 1 | Arabidopsis AP2 domain transcription factor |
| 852 | 111 | PHE0001073_1163 | 1 | Arabidopsis myb domain transcription factor |
| 853 | 112 | PHE0001075_1165 | 1 | Arabidopsis myb domain transcription factor |
| 854 | 113 | PHE0001077_1167 | 1 | Arabidopsis GARP domain transcription |
| | | | | factor |
| 855 | 114 | PHE0001083_1173 | 1 | Arabidopsis AP2 domain transcription factor |
| 856 | 115 | PHE0001095_1185 | 1 | Arabidopsis bZIP domain transcription factor |
| 857 | 116 | PHE0001096_1186 | 1 | Arabidopsis bZIP domain transcription factor |
| 858 | 117 | PHE0001097_1187 | 1 | Arabidopsis bZIP/TGA domain transcription |
| | | | | factor- |
| 859 | 118 | PHE0001098_1188 | 1 | Arabidopsis GATA domain transcription |
| | | | | factor |
| 860 | 119 | PHE0001099_1189 | 1 | Arabidopsis bZIP domain transcription factor |
| 861 | 120 | PHE0001101_1191 | 1 | Arabidopsis GATA domain transcription |
| | | | | factor |
| 862 | 121 | PHE0001107_1197 | 1 | rice CCA1-like 1 |
| 863 | 122 | PHE0001108_1198 | 1 | rice alanine aminotransferase 2-AAK52114 |
| 864 | 123 | PHE0001109_1199 | 1 | corn alanine aminotransferase 1 |
| 865 | 124 | PHE0001110_1200 | 1 | corn alanine aminotransferase 4 |
| 866 | 125 | PHE0001115_1205 | 5 | yeast 5-aminolevulinic acid synthase mature |
| | | | | form-P09950 |
| 867 | 126 | PHE0001120_1210 | 4 | corn G1820-like 1 |
| 868 | 127 | PHE0001123_1213 | 1 | corn G1820-like 2 |
| 869 | 128 | PHE0001125_1215 | 1 | soy G1820 like 2 |
| 870 | 129 | PHE0001126_1216 | 1 | corn putative myb-related transcription factor |
| 871 | 130 | PHE0001127_1217 | 1 | corn putative dehydration-responsive protein |
| | | | | RD22 precursor- |
| 872 | 131 | PHE0001128_1218 | 1 | corn homeodomain leucine zipper protein |
| 873 | 132 | PHE0001130_1220 | 5 | corn acetohydroxyacid reductoisomerase, |
| | | | | chloroplast precursor- |
| 874 | 133 | PHE0001132_1222 | 1 | corn cytosolic aspartate transaminase |
| 875 | 134 | PHE0001138_1228 | 1 | corn hypothetical protein |
| 876 | 135 | PHE0001145_1235 | 1 | corn unknown protein |
| 877 | 136 | PHE0001146_1236 | 1 | corn MAP kinase kinase |
| 878 | 137 | PHE0001147_1237 | 1 | corn hypothetical protein |
| 879 | 138 | PHE0001148_1238 | 1 | corn transfactor-like protein |
| 880 | 139 | PHE0001151_1241 | 1 | corn dehydration-responsive protein RD22 |
| | | | | precursor- |
| 881 | 140 | PHE0001152_1242 | 1 | corn histone H3 |
| 882 | 141 | PHE0001153_1243 | 1 | corn calcium-dependent protein kinase |
| 883 | 142 | PHE0001154_1244 | 1 | corn histone H2B |
| 884 | 143 | PHE0001156_1246 | 1 | corn ATFP4-like |
| 885 | 144 | PHE0001157_1247 | 1 | corn unknown protein |
| 886 | 145 | PHE0001158_1248 | 1 | corn glucose-6-phosphate/phosphate- |
| | | | | translocator precursor- |
| 887 | 146 | PHE0001159_1249 | 1 | corn DnaJ-like protein |
| 888 | 147 | PHE0001167_1257 | 1 | rem1-Mu |
| 889 | 148 | PHE0001171_1261 | 1 | soy TU8-like 1 |
| 890 | 149 | PHE0001172_1262 | 1 | soy TU8-like protein 2 |
| 891 | 150 | PHE0001179_1269 | 1 | corn AXR2-like 2 |
| 892 | 151 | PHE0001198_1288 | 1 | soybean G175-like 2 |
| 893 | 152 | PHE0001199_1289 | 1 | soybean G175-like 1 |
| 894 | 153 | PHE0001208_1298 | 1 | soybean G22-like 1 |
| 895 | 154 | PHE0001211_1301 | 1 | soybean G867-like 1 |
| 896 | 155 | PHE0001212_1302 | 1 | STE11-NP_013466 |
| 897 | 156 | PHE0001214_1304 | 1 | soybean STE11-like 1 |
| 898 | 157 | PHE0001215_1305 | 1 | soybean G1836-like 1 |
| 899 | 158 | PHE0001220_1310 | 1 | soybean AtGSK3-like 2 |
| 900 | 159 | PHE0001221_1311 | 1 | maize AtGSK3-like 1 |
| 901 | 160 | PHE0001225_1315 | 1 | maize catalase-like 1 |
| 902 | 161 | PHE0001238_1328 | 1 | maize cup1a-like 1 |
| 903 | 162 | PHE0001239_1329 | 1 | maize AtGSK3-like 2 |
| 904 | 163 | PHE0001241_1331 | 1 | Putative RNA Binding protein- |
| 905 | 164 | PHE0001242_1332 | 1 | Protein Disulfide Isomerase- |
| 906 | 165 | PHE0001244_1334 | 1 | LIM DOmain Transcription Factor SF3- |
| 907 | 166 | PHE0001245_1335 | 1 | Unkown Protein |
| 908 | 167 | PHE0001246_1337 | 1 | soy CIP8-like 1 |
| 909 | 168 | PHE0001254_1345 | 1 | Arabidopsis hypothetical protein |
| | | | | [NM_111482] |
| 910 | 169 | PHE0001258_1349 | 1 | corn NADH:ubiquinone oxidoreductase |
| 911 | 170 | PHE0001259_1350 | 1 | soy NADH:ubiquinone oxidoreductase |
| 912 | 171 | PHE0001279_1369 | 1 | corn cysteine proteinase inhibitor |
| 913 | 172 | PHE0001280_1370 | 1 | soy cysteine proteinase inhibitor |
| 914 | 173 | PHE0001282_1372 | 1 | corn fatty acid elongase 1 |
| 915 | 174 | PHE0001284_1374 | 1 | soy ADP-ribosylation factor 1 GTPase |
| | | | | activating protein- |
| 916 | 175 | PHE0001285_1375 | 1 | corn ADP-ribosylation factor 1 GTPase |
| | | | | activating protein- |
| 917 | 176 | PHE0001291_1381 | 1 | soy hypothetical protein |
| 918 | 177 | PHE0001292_1382 | 1 | corn hypothetical protein |
| 919 | 178 | PHE0001297_1387 | 1 | corn cryptochrome like protein 1 |
| 920 | 179 | PHE0001299_1389 | 1 | corn cryptochrome like protein 5 |
| 921 | 180 | PHE0001303_1393 | 1 | rice Pra2-like protein 2 |
| 922 | 181 | PHE0001306_1396 | 1 | soy Pra2-like protein 1 |
| 923 | 182 | PHE0001312_1402 | 1 | corn CGPG 1145-like protein 1 |
| 924 | 183 | PHE0001313_1403 | 1 | corn CGPG 1145-like protein 2 |
| 925 | 184 | PHE0001314_1404 | 1 | corn CGPG 1145-like protein 3 |
| 926 | 185 | PHE0001332_1423 | 1 | rice SNF1-like protein 4-BAB61199 |
| 927 | 186 | PHE0001333_1424 | 1 | rice SNF1-like protein 5-BAA96628 |
| 928 | 187 | PHE0001334_1425 | 1 | rice SNF1-like protein 6 [OSK1]-D82039 |
| 929 | 188 | PHE0001337_1428 | 1 | rice AKIN-like protein 1 |
| 930 | 189 | PHE0001339_1430 | 1 | soy AKIN-like protein 1 |
| 931 | 190 | PHE0001343_1434 | 1 | soy SNF1-like protein 3 |
| 932 | 191 | PHE0001346_1437 | 1 | corn SNF1-like protein 1 |
| 933 | 192 | PHE0001347_1438 | 1 | corn SNF1-like protein 2 |
| 934 | 193 | PHE0001349_1440 | 1 | corn SNF1-like protein 4 |
| 935 | 194 | PHE0001350_1441 | 1 | corn SNF1-like protein 5 |
| 936 | 195 | PHE0001354_1445 | 1 | corn SNF1-like protein 9 |
| 937 | 196 | PHE0001355_1446 | 1 | corn SNF1-like protein 10 |
| 938 | 197 | PHE0001356_1447 | 1 | corn SNF1-like protein 11 |
| 939 | 198 | PHE0001357_1448 | 1 | corn SNF1-like protein 13 |
| 940 | 199 | PHE0001358_1449 | 1 | corn SNF1-like protein 14 |
| 941 | 200 | PHE0001359_1450 | 1 | corn SNF1-like protein 15 |
| 942 | 201 | PHE0001360_1451 | 1 | Corn Putative CCR4 Associated Factor |
| 943 | 202 | PHE0001361_1452 | 1 | Soy Putative AP2 Domain Transcription |
| | | | | Factor |
| 944 | 203 | PHE0001362_1453 | 1 | Soy GATA Binding Transcription Factor |
| 945 | 204 | PHE0001363_1455 | 1 | NAC1 type transcriptional activator- |
| 946 | 205 | PHE0001364_1456 | 1 | Corn Putative Transcriptional Regulator X2 |
| 947 | 206 | PHE0001375_1467 | 1 | Corn PRL 1 |
| 948 | 207 | PHE0001377_1469 | 1 | rice PINOID-like protein kinase 1 sequence- |
| 949 | 208 | PHE0001380_1472 | 1 | rice PP2A regulatory subunit A RCN1-like 1 |
| | | | | sequence- |
| 950 | 209 | PHE0001385_1477 | 1 | maize IAA-alanine resistance protein IAR1- |
| | | | | like sequence- |
| 951 | 210 | PHE0001386_1478 | 1 | maize IAA-Ala hydrolase like 1 sequence- |
| 952 | 211 | PHE0001389_1481 | 1 | maize nitrilase 1 like 1 sequence- |
| 953 | 212 | PHE0001392_1484 | 1 | soybean root-specific kinase ARSK1 like |
| | | | | sequence- |
| 954 | 213 | PHE0001394_1486 | 1 | soybean MADS-box protein AGL14-like |
| | | | | sequence- |
| 955 | 214 | PHE0001397_1489 | 1 | soybean Ran binding protein RanBP1-like 1 |
| | | | | sequence- |
| 956 | 215 | PHE0001398_1490 | 1 | maize Ran binding protein RanBP1-like 1 |
| | | | | sequence- |
| 957 | 216 | PHE0001405_1497 | 1 | nifU like protein Homolog- |
| 958 | 217 | PHE0001411_1503 | 1 | photosystem-I PSI-F subunit precursor- |
| 959 | 218 | PHE0001416_1508 | 1 | growth-on protein GRO10- |
| 960 | 219 | PHE0001417_1509 | 1 | Transcription Factor Homolog BTF3- |
| 961 | 220 | PHE0001418_1510 | 1 | Putative CGI-19 Protein Homolog- |
| 962 | 221 | PHE0001421_1513 | 1 | glutamate dehydrogenase- |
| 963 | 222 | PHE0001426_1518 | 1 | Rice GATA Factor Homolog- |
| 964 | 223 | PHE0001436_1528 | 1 | Methionine Synthase- |
| 965 | 224 | PHE0001437_1529 | 1 | Putative ABC Transporter- |
| 966 | 225 | PHE0001442_1534 | 1 | corn C-24 sterol methyltransferase |
| 967 | 226 | PHE0001444_1536 | 1 | soy hypothetical protein |
| 968 | 227 | PHE0001452_1544 | 1 | maize root hair Ted2-like sequence- |
| 969 | 228 | PHE0001463_1555 | 1 | soybean homeodomain protein (GLABRA2) |
| | | | | like sequence- |
| 970 | 229 | PHE0001477_1569 | 1 | soybean werewolf (WER) like sequence- |
| 971 | 230 | PHE0001481_1573 | 1 | rice ethylene-response ETR1 like sequence- |
| 972 | 231 | PHE0001483_1575 | 1 | soybean xyloglucan endotransglycosylase |
| | | | | (XET) like 1 sequence- |
| 973 | 232 | PHE0001516_1607 | 1 | Zea Mays SelR Domain protein |
| 974 | 233 | PHE0001522_1613 | 1 | corn LPE1-like permease 6 |
| 975 | 234 | PHE0001539_1630 | 1 | soy glutamate dehydrogenase |
| 976 | 235 | PHE0001577_1676 | 1 | Rice Homolog Putative Transcription Factor |
| | | | | X2- |
| 977 | 236 | PHE0001602_1713 | 1 | rice rac-like GTP binding protein like 1 |
| | | | | sequence |
| 978 | 237 | PHE0001604_1715 | 1 | rice rac-like GTP binding protein like 3 |
| | | | | sequence |
| 979 | 238 | PHE0001605_1716 | 1 | rice rac-like GTP binding protein like 4 |
| | | | | sequence |
| 980 | 239 | PHE0001606_1717 | 1 | rice rac-like GTP binding protein like 5 |
| | | | | sequence |
| 981 | 240 | PHE0001610_1721 | 1 | maize translation initiation factor 3 delta |
| | | | | subunit like sequence |
| 982 | 241 | PHE0001617_1728 | 1 | maize metal transporter ZIP8 like 1 sequence |
| 983 | 242 | PHE0001624_1735 | 1 | maize magnesium transporter, mrs2-2-like 1 |
| | | | | sequence |
| 984 | 243 | PHE0001629_1740 | 1 | maize low temperature and salt responsive |
| | | | | protein LT16A-like 1 sequence |
| 985 | 244 | PHE0001642_1753 | 1 | rice salt-induced-zinc-finger-protein 1 |
| | | | | sequence |
| 986 | 245 | PHE0001649_1760 | 1 | soybean salt-tolerance protein 1 seqeunce |
| 987 | 246 | PHE0002017_2128 | 1 | Corn RING Finger Transcription Factor |
| 988 | 247 | PHE0002023_2134 | 1 | Corn RING Finger Transcription Factor II |
| 989 | 248 | PHE0002024_2135 | 1 | Corn RING Finger Transcription Factor III |
| 990 | 249 | PHE0002027_2138 | 1 | Glutamine Synthetase |
| 991 | 250 | PHE0002041_2151 | 1 | Corn ubiquitin fusion protein/ribosomal |
| | | | | protein S27a |
| 992 | 251 | PHE0002042_2152 | 1 | soybean AtHSP17.6A like 1 sequence |
| 993 | 252 | PHE0002049_2159 | 1 | Ribulose bisphosphate carboxylase small |
| | | | | chain chloroplast precursor |
| 994 | 253 | PHE0002052_2162 | 1 | Corn ligand-gated channel-like protein |
| | | | | precursor |
| 995 | 254 | PHE0002054_2164 | 1 | Corn Nitrilase 1 |
| 996 | 255 | PHE0002055_2165 | 1 | Corn Tic 22 Like protein |
| 997 | 256 | PHE0002067_2177 | 1 | corn G664-like 5 |
| 998 | 257 | PHE0002068_2178 | 1 | corn G664-like 6 |
| 999 | 258 | PHE0002071_2181 | 1 | soy G664-like 2 |
| 1000 | 259 | PHE0002080_4290 | 11 | corn sec61 |
| 1001 | 260 | PHE0002081_2191 | 1 | soy sec61 |
| 1002 | 261 | PHE0002088_2198 | 1 | rice osr8 |
| 1003 | 262 | PHE0002095_2205 | 1 | Corn Calmodulin EF hand |
| 1004 | 263 | PHE0002096_2206 | 1 | Rice Calmodulin EF Hand |
| 1005 | 264 | PHE0002099_2209 | 1 | Rice Putative Calmodulin EF Hand Protein |
| 1006 | 265 | PHE0002103_2213 | 1 | rice Bobs kinase 2 |
| 1007 | 266 | PHE0002123_2231 | 4 | rice phosphoenolpyruvate carboxylase 2 |
| 1008 | 267 | PHE0002125_2233 | 1 | corn phosphoenolpyruvate carboxylase 2 |
| 1009 | 268 | PHE0002127_2235 | 1 | soy phosphoenolpyruvate carboxylase 2 |
| 1010 | 269 | PHE0002145_2253 | 1 | Corn Unkown Protein |
| 1011 | 270 | PHE0002148_2256 | 1 | Corn Cytochrome P450 |
| 1012 | 271 | PHE0002163_2270 | 1 | Corn Protein (Similar to peptidyl prolyl |
| | | | | isomerase) |
| 1013 | 272 | PHE0002164_2271 | 1 | Corn Protein (similar to yippie-1 & a peptidyl |
| | | | | prolyl isomerase) |
| 1014 | 273 | PHE0002183_2290 | 1 | soybean Hsp17.7 like 1 sequence |
| 1015 | 274 | PHE0002189_2296 | 1 | maize pyrroline-5-carboxylate synthetase like |
| | | | | 2 sequence |
| 1016 | 275 | PHE0002202_2309 | 1 | soy annexin 1 |
| 1017 | 276 | PHE0002203_2310 | 1 | soy annexin 3 |
| 1018 | 277 | PHE0002204_2311 | 1 | soy annexin 4 |
| 1019 | 278 | PHE0002210_2317 | 1 | soybean drought-induced protein BnD22 like |
| | | | | 1 sequence |
| 1020 | 279 | PHE0002224_2331 | 1 | maize drought-induced protein Di19-like |
| | | | | sequence |
| 1021 | 280 | PHE0002227_2334 | 1 | maize protease inhibitor like 2 sequence |
| 1022 | 281 | PHE0002232_2339 | 1 | soybean ABA-responsive element binding |
| | | | | protein 2 (AREB2) like sequence |
| 1023 | 282 | PHE0002233_2340 | 1 | Arabidopsis ABA-responsive element binding |
| | | | | protein 3 (AREB3) like sequence |
| 1024 | 283 | PHE0002238_2345 | 1 | soybean CPRD14 like 1 sequence |
| 1025 | 284 | PHE0002239_2346 | 1 | Brassica napus CPRD14 like 1 sequence |
| 1026 | 285 | PHE0002240_2347 | 1 | soybean CPRD14 like 2 sequence |
| 1027 | 286 | PHE0002242_2349 | 1 | maize CPRD14 like 1 sequence |
| 1028 | 287 | PHE0002244_2351 | 1 | Arabidopsis drought inducible heat shock |
| | | | | transcription factor like sequence |
| 1029 | 288 | PHE0002246_2353 | 1 | soybean protein phosphatase 2C like sequence |
| 1030 | 289 | PHE0002254_2361 | 1 | soybean CPRD12 like 3 sequence |
| 1031 | 290 | PHE0002268_2371 | 1 | soybean calcium-dependent protein kinase |
| | | | | like 1 sequence |
| 1032 | 291 | PHE0002269_2372 | 1 | rice calcium-dependent protein kinase like 1 |
| | | | | sequence |
| 1033 | 292 | PHE0002272_2375 | 1 | rice calcium-dependent protein kinase like 4 |
| | | | | sequence |
| 1034 | 293 | PHE0002298_2400 | 4 | rice Dehydrin ERD10 like 1 sequence |
| 1035 | 294 | PHE0002299_2401 | 1 | rice drought-induced S-like ribonuclease like |
| | | | | 1 sequence |
| 1036 | 295 | PHE0002321_2422 | 1 | maize Dil9 like sequence |
| 1037 | 296 | PHE0002331_2432 | 1 | soybean PIP like 1 sequence |
| 1038 | 297 | PHE0002340_2441 | 1 | soybean MIP4 like 1 sequence |
| 1039 | 298 | PHE0002354_2455 | 1 | maize glutathione transferase like 2 sequence |
| 1040 | 299 | PHE0002357_2458 | 1 | maize 8-oxoguanine-DNA glycosylase like |
| | | | | sequence |
| 1041 | 300 | PHE0002361_2462 | 1 | rice CEO1 like sequence |
| 1042 | 301 | PHE0002362_2463 | 1 | maize dolichyl-phosphate beta- |
| | | | | glucosyltransferase like sequence |
| 1043 | 302 | PHE0002364_2465 | 1 | maize glutathione reductase (GR2) like 1 |
| | | | | sequence |
| 1044 | 303 | PHE0002365_2466 | 1 | soybean glutathione reductase (GR2) like 1 |
| | | | | sequence |
| 1045 | 304 | PHE0002367_2468 | 1 | rice glutathione reductase (GR2) like 1 |
| | | | | sequence |
| 1046 | 305 | PHE0002370_2471 | 1 | soybean glutathione-peroxidase like 1 |
| | | | | sequence |
| 1047 | 306 | PHE0002372_2473 | 1 | maize glutathione-peroxidase like 2 sequence |
| 1048 | 307 | PHE0002373_2474 | 1 | maize glutathione-peroxidase like 3 sequence |
| 1049 | 308 | PHE0002383_2484 | 1 | maize T-complex polypeptide 1 alpha subunit |
| | | | | like sequence |
| 1050 | 309 | PHE0002390_2491 | 1 | soybean methionine sulfoxide reductase (msr) |
| | | | | like sequence |
| 1051 | 310 | PHE0002414_2514 | 1 | Arabidopsis GAD2 |
| 1052 | 311 | PHE0002431_2531 | 1 | Rice Leucine Zipper Protein similar to At103 |
| | | | | and PNIL34 |
| 1053 | 312 | PHE0002447_2547 | 1 | soybean arabidopsis-heat-shock-TF like 1 |
| | | | | sequence |
| 1054 | 313 | PHE0002449_2549 | 1 | soybean heat shock factor 6 like 1 sequence |
| 1055 | 314 | PHE0002459_2559 | 1 | rice heat shock TF like sequence |
| 1056 | 315 | PHE0002461_2561 | 1 | soybean HSF1 like 1 sequence |
| 1057 | 316 | PHE0002462_2562 | 1 | soybean HSF4 like 1 sequence |
| 1058 | 317 | PHE0002464_2564 | 1 | soybean HSF4 like 3 sequence |
| 1059 | 318 | PHE0002470_2570 | 1 | soybean hsp17.4 like 2 sequence |
| 1060 | 319 | PHE0002471_2571 | 1 | soybean hsp17.4 like 3 sequence |
| 1061 | 320 | PHE0002484_2584 | 1 | Corn Sucrose Synthase |
| 1062 | 321 | PHE0002486_2586 | 1 | Corn Putative protein phosphatase 2A |
| | | | | regulatory subunit B |
| 1063 | 322 | PHE0002489_2589 | 1 | Arabidopsis CycD2 |
| 1064 | 323 | PHE0002490_2590 | 1 | Arabidopsis CycD3 |
| 1065 | 324 | PHE0002490_3963 | 16 | Arabidopsis CycD3 |
| 1066 | 325 | PHE0002491_2591 | 1 | Arabidopsis CycB1 |
| 1067 | 326 | PHE0002498_2598 | 1 | Corn Protein Kinase like protein |
| 1068 | 327 | PHE0002502_2602 | 1 | Corn 20 KDA CHAPERONIN, |
| | | | | CHLOROPLAST PRECURSOR (PROTEIN |
| | | | | CPN21) |
| 1069 | 328 | PHE0002506_2606 | 1 | Corn protein of unknown function wi PFAM |
| | | | | domain similar to human Reticulon and may |
| | | | | associate with ER |
| 1070 | 329 | PHE0002507_2607 | 1 | Corn Transfactor like protein |
| 1071 | 330 | PHE0002514_2614 | 1 | Corn branched-chain alpha keto-acid |
| | | | | dehydrogenase E1 alpha subunit |
| 1072 | 331 | PHE0002515_2615 | 1 | Corn haloacid dehalogenase-like hydrolase |
| | | | | Putative ripening related protein |
| 1073 | 332 | PHE0002530_2630 | 1 | Rice Dof zinc finger protein |
| 1074 | 333 | PHE0002532_2632 | 1 | Corn ribosomal protein L1p/L10e family |
| 1075 | 334 | PHE0002536_2636 | 1 | Rice auxin response transcription factor 3-like |
| | | | | protein |
| 1076 | 335 | PHE0002540_2640 | 1 | Oryza Sativa auxin response factor 2 |
| 1077 | 336 | PHE0002547_2647 | 1 | Corn homolog to Arabidopsis protein wi |
| | | | | hydrolase fold |
| 1078 | 337 | PHE0002551_2651 | 1 | Corn TF with 3 leucine zippers & PFAM |
| | | | | mTERF domain |
| 1079 | 338 | PHE0002552_2652 | 1 | Corn protein with Duf210 PFAM domain |
| 1080 | 339 | PHE0002565_2664 | 1 | Corn metallothionein-like protein |
| 1081 | 340 | PHE0002581_2680 | 1 | maize hsp60 like 1 sequence |
| 1082 | 341 | PHE0002582_2681 | 1 | maize hsp60 like 2 sequence |
| 1083 | 342 | PHE0002583_2682 | 1 | maize hsp60 like 3 sequence |
| 1084 | 343 | PHE0002586_2685 | 1 | rice hsp60 like 1 sequence |
| 1085 | 344 | PHE0002588_2687 | 1 | Corn protein with “Universal Stress Protein |
| | | | | Family” PFAM domain |
| 1086 | 345 | PHE0002591_2690 | 1 | Corn protein similar to Yersinia pestis |
| | | | | membrane protein and Bacillus Subtilis yuxK |
| 1087 | 346 | PHE0002596_2695 | 1 | Corn Myb related Transcription Factor |
| 1088 | 347 | PHE0002604_2703 | 1 | Rice putative putative photoreceptor- |
| | | | | interacting protein-like protein |
| 1089 | 348 | PHE0002608_2707 | 1 | Arabidopsis sigma factor 2 |
| 1090 | 349 | PHE0002615_2714 | 1 | corn sigA binding protein 2 |
| 1091 | 350 | PHE0002622_2739 | 1 | Arabidopsis GLK1 |
| 1092 | 351 | PHE0002629_2748 | 1 | rice trehalose-6-P synthase like 1 sequence |
| 1093 | 352 | PHE0002634_2753 | 1 | rice DL-glycerol-3-phosphatase like 1 |
| | | | | sequence |
| 1094 | 353 | PHE0002639_2758 | 1 | rice GH3 protein 8 |
| 1095 | 354 | PHE0002640_2759 | 1 | rice GH3 protein 3 |
| 1096 | 355 | PHE0002643_2762 | 1 | rice GH3 protein 2 |
| 1097 | 356 | PHE0002644_2763 | 1 | rice GH3 protein 9 |
| 1098 | 357 | PHE0002649_2768 | 1 | wheat AGL21-like 1 |
| 1099 | 358 | PHE0002661_2781 | 5 | Synechocystis sp. PCC 6803 ADP-glucose |
| | | | | pyrophosphorylase |
| 1100 | 359 | PHE0002664_2787 | 6 | rice ADP-glucose pyrophosphorylase 5 |
| 1101 | 360 | PHE0002688_2821 | 1 | rice beta-3 tubulin like 1 sequence |
| 1102 | 361 | PHE0002689_2822 | 1 | rice beta-3 tubulin like 2 sequence |
| 1103 | 362 | PHE0002690_2823 | 1 | Corn protein similar to cell division related |
| | | | | protein kinase |
| 1104 | 363 | PHE0002703_2836 | 1 | rice VTC2 like 1 sequence |
| 1105 | 364 | PHE0002710_2843 | 1 | Zea Mays cytoplasmic malate dehydrogenase |
| 1106 | 365 | PHE0002715_2848 | 1 | Oryza sativa putative thiolase |
| 1107 | 366 | PHE0002717_2850 | 1 | Corn Translation Elongation factor EF1-beta |
| 1108 | 367 | PHE0002721_2854 | 4 | Maize fructose-bisphosphate aldolase |
| 1109 | 368 | PHE0002724_2859 | 1 | Corn L19 Like ribosomal protein |
| 1110 | 369 | PHE0002728_2861 | 1 | maize sucrose transport protein SUC2 like 1 |
| | | | | sequence |
| 1111 | 370 | PHE0002729_2863 | 1 | Corn 60S ribosomal protein L10 (probable |
| | | | | transcription factor) |
| 1112 | 371 | PHE0002733_2866 | 1 | Corn Ribosomal protein S11 |
| 1113 | 372 | PHE0002734_2867 | 1 | Corn Ribosomal protein S12 |
| 1114 | 373 | PHE0002749_2882 | 1 | rice sucrase-like 1 sequence |
| 1115 | 374 | PHE0002751_2884 | 1 | Corn Ribosomal protein S14 |
| 1116 | 375 | PHE0002752_2885 | 1 | Corn Homolog to putative 40S Ribosomal |
| | | | | protein |
| 1117 | 376 | PHE0002771_2904 | 1 | [Zea mays] beta-glucosidase aggregating |
| | | | | factor precursor |
| 1118 | 377 | PHE0002790_2925 | 1 | rice CYP72A5 like 1 sequence |
| 1119 | 378 | PHE0002846_2981 | 1 | Zea Mays trehalose-6-phosphate phosphatase |
| 1120 | 379 | PHE0002864_2999 | 1 | soy CDKA 8 |
| 1121 | 380 | PHE0002869_3004 | 1 | corn CDKD 12 |
| 1122 | 381 | PHE0002875_3010 | 1 | Corn homolog to Arabidopsis unknown |
| | | | | expressed protein |
| 1123 | 382 | PHE0002889_3024 | 1 | soy dsPTP 3 |
| 1124 | 383 | PHE0002896_3031 | 1 | rice dsPTP 1 |
| 1125 | 384 | PHE0002918_3053 | 1 | Oryza Sativa putative Hexose Transporter IV |
| | | | | (distant homology to Yeast Maltose |
| | | | | permease) |
| 1126 | 385 | PHE0002946_3096 | 1 | Zea mays Putative Polyamine Transporter |
| 1127 | 386 | PHE0002963_3113 | 1 | Zea Mays Dual Specificity Phosphatase I |
| | | | | (similar to human YVH1) |
| 1128 | 387 | PHE0002966_3116 | 1 | Oryza Sativa Dual Specificity Phosphatase II |
| 1129 | 388 | PHE0002984_3134 | 1 | Zea mays 3-phosphoinositide-dependent |
| | | | | protein kinase |
| 1130 | 389 | PHE0003061_3211 | 1 | [Oryza sativa] Putative integral membrane |
| | | | | protein |
| 1131 | 390 | PHE0003074_3224 | 1 | Zea Mays RNA Binding Protein |
| 1132 | 391 | PHE0003101_3969 | 16 | soy nonsymbiotic hemoglobin |
| 1133 | 392 | PHE0003101_76 | 3 | soy nonsymbiotic hemoglobin |
| 1134 | 393 | PHE0003124_3270 | 1 | soy adenylylsulfate kinase like |
| 1135 | 394 | PHE0003138_3292 | 6 | rice fructokinase 1 |
| 1136 | 395 | PHE0003139_3295 | 1 | corn fructokinase 1 |
| 1137 | 396 | PHE0003190_3389 | 1 | soy G1225-like 2 |
| 1138 | 397 | PHE0003196_3395 | 1 | Arabidopsis seven-in-absentia 1 |
| 1139 | 398 | PHE0003198_3397 | 1 | corn seven-in-absentia 5 |
| 1140 | 399 | PHE0003211_3417 | 4 | Arabidopsis AVP1 |
| 1141 | 400 | PHE0003211_3967 | 16 | Arabidopsis AVP1 |
| 1142 | 401 | PHE0003217_3423 | 1 | soy G200-like 13 |
| 1143 | 402 | PHE0003224_3432 | 1 | corn-CGPG1897-like5 |
| 1144 | 403 | PHE0003228_3444 | 16 | soy SAG13 full length |
| 1145 | 404 | PHE0003229_3445 | 16 | soy SAG13 truncated |
| 1146 | 405 | PHE0003237_3453 | 1 | rice-CGPG1264-like1 |
| 1147 | 406 | PHE0003240_3456 | 1 | soy-CGPG1857-like1 |
| 1148 | 407 | PHE0003243_3459 | 1 | corn-CGPG867-like4 |
| 1149 | 408 | PHE0003253_3470 | 1 | soy-CGPG1276-like1 |
| 1150 | 409 | PHE0003257_3474 | 1 | soy-CGPG1294-like2 |
| 1151 | 410 | PHE0003273_3492 | 1 | soy-CGPG1287-like1 |
| 1152 | 411 | PHE0003276_3495 | 1 | Arabidopsis dehydrogenase |
| 1153 | 412 | PHE0003282_3501 | 1 | corn-CGPG241-like3[DUF6] |
| 1154 | 413 | PHE0003286_3505 | 1 | rice CTP synthase |
| 1155 | 414 | PHE0003287_3506 | 1 | corn CTP synthase |
| 1156 | 415 | PHE0003304_3524 | 6 | corn unknown protein |
| 1157 | 416 | PHE0003309_3528 | 1 | soy-CGPG1307-like1 |
| 1158 | 417 | PHE0003312_3531 | 1 | Arabidopsis nitrate transporter |
| 1159 | 418 | PHE0003321_3540 | 1 | Arabidopsis PDK1 like 2 |
| 1160 | 419 | PHE0003327_3546 | 1 | corn NEK like-1 |
| 1161 | 420 | PHE0003328_3547 | 1 | corn NEK like-2 |
| 1162 | 421 | PHE0003330_3549 | 1 | Arabidopsis eIFiso4E like p28 subunit |
| 1163 | 422 | PHE0003333_3552 | 1 | soy eIF4E like 1 |
| 1164 | 423 | PHE0003333_4250 | 17 | soy eIF4E like 1 |
| 1165 | 424 | PHE0003336_3555 | 1 | Arabidopsis eIF4E |
| 1166 | 425 | PHE0003353_3572 | 1 | corn seven-in-absentia 5 C57S |
| 1167 | 426 | PHE0003361_3584 | 1 | malate dehydrogenase |
| 1168 | 427 | PHE0003362_3585 | 1 | hexokinase |
| 1169 | 428 | PHE0003364_3587 | 1 | aminotransferase-like protein |
| 1170 | 429 | PHE0003369_3592 | 4 | glyceraldehyde 3-phosphate dehydrogenase |
| 1171 | 430 | PHE0003373_3596 | 1 | putative purple acid phosphatase |
| 1172 | 431 | PHE0003381_3604 | 1 | hypothetical protein2 |
| 1173 | 432 | PHE0003385_3608 | 1 | expressed protein2 |
| 1174 | 433 | PHE0003391_3614 | 1 | serine/threonine protein kinase like protein |
| 1175 | 434 | PHE0003392_3615 | 1 | lipase-like protein |
| 1176 | 435 | PHE0003393_3616 | 1 | Putative Squalene monooxygenase |
| 1177 | 436 | PHE0000132_222 | 2 | FUS5 |
| 1178 | 437 | PHE0000285_375 | 1 | sorghum mLIP15 |
| 1179 | 438 | PHE0000668_771 | 1 | Arabidopsis glycerol-3-phosphate |
| | | | | acyltransferase-D00673 |
| 1180 | 439 | PHE0000790_863 | 1 | soy agl1 1-like 1 delta MADS-box |
| 1181 | 440 | PHE0001074_1164 | 1 | Arabidopsis NAC domain transcription factor |
| 1182 | 441 | PHE0001078_1168 | 1 | Arabidopsis NAC domain transcription factor |
| 1183 | 442 | PHE0001079_1169 | 1 | Arabidopsis NAC domain transcription factor |
| 1184 | 443 | PHE0001082_1172 | 1 | Arabidopsis NAC domain transcription factor |
| 1185 | 444 | PHE0001085_1175 | 1 | Arabidopsis myc domain transcription factor |
| 1186 | 445 | PHE0001113_1203 | 1 | Arabidopsis serine glyoxylate |
| | | | | aminotransferase-AB048945 |
| 1187 | 446 | PHE0001136_1226 | 1 | rice aspartate aminotransferase-D14673 |
| 1188 | 447 | PHE0001142_1232 | 1 | corn caffeoyl-CoA 3-O-methyltransferase |
| 1189 | 448 | PHE0001216_1306 | 1 | maize G1836-like 1 |
| 1190 | 449 | PHE0001240_1330 | 1 | Inorganic pyrophosphatase- |
| 1191 | 450 | PHE0001243_1333 | 1 | Tubby Protein- |
| 1192 | 451 | PHE0001248_1339 | 1 | Arabidopsis hypothetical protein |
| 1193 | 452 | PHE0001257_1348 | 1 | Arabidopsis NADH:ubiquinone |
| | | | | oxidoreductase |
| 1194 | 453 | PHE0001263_1354 | 1 | Arabidopsis hypothetical protein |
| | | | | [NM_114619] |
| 1195 | 454 | PHE0001266_1357 | 1 | corn hypothetical protein |
| 1196 | 455 | PHE0001283_1373 | 1 | Arabidopsis Asp1 |
| 1197 | 456 | PHE0001286_1376 | 1 | Arabidopsis putative inositol hexaphosphate |
| | | | | kinase- |
| 1198 | 457 | PHE0001301_1391 | 1 | Arabidopsis Pra2-like protein-AAF97325 |
| 1199 | 458 | PHE0001304_1394 | 1 | corn Pra2-like protein 1 |
| 1200 | 459 | PHE0001316_1406 | 1 | rice CGPG 1145-like protein 1 |
| 1201 | 460 | PHE0001317_1407 | 1 | rice CGPG 1145-like protein 2 |
| 1202 | 461 | PHE0001319_1409 | 1 | Arabidopsis hypothetical protein |
| 1203 | 462 | PHE0001323_1413 | 1 | Arabidopsis hypothetical protein |
| | | | | [NM_111447] |
| 1204 | 463 | PHE0001324_1414 | 1 | Arabidopsis expressed protein |
| 1205 | 464 | PHE0001466_1558 | 1 | Arabidopsis thaliana ROOT HAIRLESS 1 |
| | | | | (RHL1)-AAC23500.1 |
| 1206 | 465 | PHE0001564_1663 | 4 | Xanthomonas campestris asparagine synthase |
| 1207 | 466 | PHE0002094_2204 | 1 | Corn serine/threonine kinase |
| 1208 | 467 | PHE0002121_2229 | 1 | corn G664-like 4 |
| 1209 | 468 | PHE0002225_2332 | 1 | rice drought-induced protein Di19-like |
| | | | | sequence |
| 1210 | 469 | PHE0002226_2333 | 1 | maize protease inhibitor like 1 sequence |
| 1211 | 470 | PHE0002293_2395 | 1 | soybean drought-inducible cysteine proteinase |
| | | | | like 1 sequence |
| 1212 | 471 | PHE0002338_2439 | 1 | maize MIP3 like 3 sequence |
| 1213 | 472 | PHE0002524_2624 | 1 | Rice putative thioredoxin like protein |
| 1214 | 473 | PHE0002567_2666 | 1 | Arabidopsis VPE1 |
| 1215 | 474 | PHE0002607_2706 | 1 | Arabidopsis Sigma factor 1 |
| 1216 | 475 | PHE0002609_2708 | 1 | Arabidopsis sigma factor 3 |
| 1217 | 476 | PHE0002625_2744 | 1 | rice waxy like 1 sequence |
| 1218 | 477 | PHE0002939_3089 | 1 | Oryza Sativa Proline oxidase (proline |
| | | | | dehydrogenase) |
| 1219 | 478 | PHE0003057_3207 | 1 | Schizosaccharomyces pombe orb6 |
| 1220 | 479 | PHE0003182_3341 | 1 | Neurospora_crassa Glycogen synthase like |
| | | | | sequence |
| 1221 | 480 | PHE0003232_3448 | 1 | soy-CGPG1264-like3 |
| 1222 | 481 | PHE0003234_3450 | 1 | rice-CGPG1464-like3 |
| 1223 | 482 | PHE0003239_3455 | 1 | corn-CGPG1857-like2 |
| 1224 | 483 | PHE0003246_3462 | 1 | Arabidopsis hypothetical protein |
| 1225 | 484 | PHE0003259_3476 | 1 | Arabidopsis Microtubule associated protein |
| 1226 | 485 | PHE0003267_3486 | 1 | Arabidopsis Glycine-tRNA |
| 1227 | 486 | PHE0003269_3488 | 1 | Arabidopsis unknown protein |
| 1228 | 487 | PHE0003274_3493 | 1 | Arabidopsis Unknown protein |
| 1229 | 488 | PHE0003285_3504 | 1 | rice-CGPG566-like1 |
| 1230 | 489 | PHE0003289_3508 | 8 | corn aluminium-induced protein |
| 1231 | 490 | PHE0003311_3530 | 1 | Arabidopsis hypothetical protein |
| 1232 | 491 | PHE0003360_3583 | 1 | 3-ketoacyl-ACP synthase |
| 1233 | 492 | PHE0003365_3588 | 1 | putative chlorophyll synthase |
| 1234 | 493 | PHE0003374_3597 | 1 | putative xyloglucan endotransglycosylase |
| 1235 | 494 | PHE0003375_3598 | 1 | Tryptophan synthase alpha chain |
| 1236 | 495 | PHE0003378_3601 | 1 | MtN3 |
| 1237 | 496 | PHE0003379_3602 | 1 | hypothetical protein1 |
| 1238 | 497 | PHE0003383_3606 | 1 | expressed protein1 |
| 1239 | 498 | PHE0003387_3610 | 4 | nodulin-like protein |
| 1240 | 499 | PHE0003401_3624 | 1 | corn sterol 5 alpha desaturase |
| 1241 | 500 | PHE0003417_3657 | 19 | soy G1634-like 1 |
| 1242 | 501 | PHE0003418_3658 | 19 | Arabidopsis isocitrate lyase |
| 1243 | 502 | PHE0003433_3642 | 1 | corn dehalogenase-phosphatase 1 |
| 1244 | 503 | PHE0003450_3681 | 1 | soy G1274 like 1 |
| 1245 | 504 | PHE0003458_3689 | 1 | corn G1660 like 1 |
| 1246 | 505 | PHE0003459_3690 | 1 | corn G1730 like 1 |
| 1247 | 506 | PHE0003476_3707 | 1 | soy G1988 like 1 |
| 1248 | 507 | PHE0003484_3730 | 1 | corn G2035 like1 |
| 1249 | 508 | PHE0003491_3737 | 1 | soy G2063 like2 |
| 1250 | 509 | PHE0003499_3745 | 1 | rice G2207 like2 |
| 1251 | 510 | PHE0003518_3764 | 1 | soy G2505 like1 |
| 1252 | 511 | PHE0003524_3770 | 1 | soy G2536 like1 |
| 1253 | 512 | PHE0003570_3816 | 1 | soy G922 like1 |
| 1254 | 513 | PHE0003576_3822 | 1 | soy G975 like1 |
| 1255 | 514 | PHE0003583_3829 | 1 | corn G728 like1 |
| 1256 | 515 | PHE0003587_3833 | 1 | soy G3083 like1 |
| 1257 | 516 | PHE0003599_3845 | 1 | corn G2981 like1 |
| 1258 | 517 | PHE0003600_3846 | 4 | corn R-S |
| 1259 | 518 | PHE0003630_3888 | 1 | corn G1206 like 2 |
| 1260 | 519 | PHE0003641_3899 | 4 | corn G2998 like2 |
| 1261 | 520 | PHE0003644_3902 | 1 | corn G303 like2 |
| 1262 | 521 | PHE0003650_3908 | 1 | soy G355 like2 |
| 1263 | 522 | PHE0003678_3936 | 1 | corn G1052 like2 |
| 1264 | 523 | PHE0003686_3961 | 16 | soy NDPK2 |
| 1265 | 524 | PHE0003740_4042 | 16 | soy G481-like 3 |
| 1266 | 525 | PHE0003742_4044 | 4 | corn UDP-glucose 4-epimerase CGPG1003 |
| | | | | like2 |
| 1267 | 526 | PHE0003743_4046 | 4 | rice UDP-glucose 4-epimerase CGPG1003 |
| | | | | like1 |
| 1268 | 527 | PHE0003825_4192 | 4 | soy G1412 like1 |
| 1269 | 528 | PHE0003839_4208 | 4 | rice G2604 like1 |
| 1270 | 529 | PHE0003885_4266 | 4 | wheat hemoglobin |
| 1271 | 530 | PHE0003886_4267 | 4 | Arabidopsis hemoglobin-like 1 |
| 1272 | 531 | PHE0003927_4321 | 4 | Zea mays AIH-like |
| 1273 | 532 | PHE0003959_4544 | 4 | Glycine max CGPG7857 pseudo-response |
| | | | | regulator |
| 1274 | 533 | PHE0003961_4662 | 14 | maize Kas I |
| 1275 | 534 | PHE0003965_4553 | 4 | corn ABI5 like |
| 1276 | 535 | PHE0003966_4554 | 4 | Corn AtUPS1-like |
| 1277 | 536 | PHE0003977_4565 | 4 | corn AfMONFEED001213 uroporphyrinogen |
| | | | | III synthase |
| 1278 | 537 | PHE0003978_4566 | 4 | corn AfMONFEED000902 |
| | | | | proteophosphoglycan |
| 1279 | 538 | PHE0003982_4569 | 4 | corn AfMONFEED000317 serine |
| | | | | carboxypeptidase II-1 |
| 1280 | 539 | PHE0003993_4579 | 4 | corn AfMONFEED001219 putative |
| | | | | Ca2+/H+-exchanging protein |
| 1281 | 540 | PHE0003994_4580 | 4 | corn AfMONFEED001169 putative |
| | | | | pathogenesis related protein |
| 1282 | 541 | PHE0003996_4582 | 4 | corn AfMONFEED000906 auxin-induced |
| | | | | protein-related |
| 1283 | 542 | PHE0003997_4583 | 4 | corn AfMONFEED001366 isoflavone |
| | | | | reductase homolog |
| 1284 | 543 | PHE0003999_4585 | 4 | corn AfMONFEED000689 proline-rich |
| | | | | protein |
| 1285 | 544 | PHE0004000_4586 | 4 | corn AfMONFEED000920 nodulin-related |
| | | | | protein |
| 1286 | 545 | PHE0004002_4588 | 4 | corn AfMONFEED001475 hypothetical |
| | | | | protein |
| 1287 | 546 | PHE0004003_4589 | 4 | corn AfMONFEED000648 acyltransferase |
| 1288 | 547 | PHE0004004_4590 | 4 | corn AfMONFEED000503 pathogenesis- |
| | | | | related protein 4 |
| 1289 | 548 | PHE0004005_4591 | 4 | corn AfMONFEED001364 unknown protein |
| 1290 | 549 | PHE0004006_4592 | 4 | corn AfMONFEED000623 hydrolase |
| 1291 | 550 | PHE0004009_4595 | 4 | corn AfMONFEED000074 unknown protein |
| 1292 | 551 | PHE0004010_4596 | 4 | corn AfMONFEED000152 Myb-like DNA- |
| | | | | binding domain |
| 1293 | 552 | PHE0004011_4597 | 4 | corn AfMONFEED001001 ACT domain- |
| | | | | containing protein |
| 1294 | 553 | PHE0000788_861 | 4 | corn agl1 1-like 1 delta C-terminus |
| 1295 | 554 | PHE0000789_862 | 4 | corn agl1 1-like 1 delta K-box |
| 1296 | 555 | PHE0000795_868 | 1 | rice MADS3 delta K-box-L37528 |
| 1297 | 556 | PHE0000881_964 | 1 | Synechocystis sp. PCC 6803 Hik33 |
| 1298 | 557 | PHE0001226_1316 | 1 | wheat catalase-like 1 |
| 1299 | 558 | PHE0001253_1344 | 1 | soy arabinoglactan-protein 1 |
| 1300 | 559 | PHE0001335_1426 | 1 | rice SNF1-like protein 8 [OsPK7]- |
| | | | | BAA83689 |
| 1301 | 560 | PHE0001381_1473 | 1 | rice ANR1-like |
| 1302 | 561 | PHE0001521_1612 | 1 | soy LPE1-like permease 2 |
| 1303 | 562 | PHE0001630_1741 | 1 | maize low temperature and salt responsive |
| | | | | protein LTI6A-like 2 sequence |
| 1304 | 563 | PHE0001659_1770 | 1 | corn SDD1-like 1 pre-pro domain |
| 1305 | 564 | PHE0002079_2189 | 1 | rice sec61 |
| 1306 | 565 | PHE0002185_2292 | 1 | rice Hsp17.7 like 1 sequence |
| 1307 | 566 | PHE0002229_2336 | 1 | rice protease inhibitor like 1 sequence |
| 1308 | 567 | PHE0002392_2493 | 1 | rice copper chaperone like 1 sequence |
| 1309 | 568 | PHE0002410_2510 | 1 | E. coli gadA |
| 1310 | 569 | PHE0002411_2511 | 1 | Synechocystis sp. PCC 6803 gad |
| 1311 | 570 | PHE0002485_2585 | 4 | Corn Amino Acid Transport Protein |
| 1312 | 571 | PHE0002496_2596 | 1 | Corn Glutamate-1-semialdehyde |
| | | | | aminotransferase |
| 1313 | 572 | PHE0002611_2710 | 1 | soy sigma factor 1 |
| 1314 | 573 | PHE0002616_2723 | 1 | Arabidopsis proline/glycine betaine |
| | | | | transporter 1 |
| 1315 | 574 | PHE0002670_2793 | 5 | Streptomyces coelicolor glucose-1-phosphate |
| | | | | adenylyltransferase |
| 1316 | 575 | PHE0002727_2860 | 1 | rice sucrose synthase-1 like 2 sequence |
| 1317 | 576 | PHE0002950_3100 | 1 | Yeast protein involved in resistance to H2O2 |
| 1318 | 577 | PHE0002997_3147 | 1 | Yeast Protein of unknown function, has WD |
| | | | | (WD-40) repeats |
| 1319 | 578 | PHE0003011_3161 | 1 | Yeast Protein (wi Carbonic anhydrase PFAM |
| | | | | domain) involved in protection against |
| | | | | oxidative damage |
| 1320 | 579 | PHE0003021_3171 | 1 | Yeast protein of unknown function (may be |
| | | | | involved in cell damage) |
| 1321 | 580 | PHE0003200_3399 | 1 | Arabidopsis seven-in-absentia 2 |
| 1322 | 581 | PHE0003227_3443 | 16 | soy G1820 like |
| 1323 | 582 | PHE0003261_3478 | 1 | rice-CGPG1517-like2 |
| 1324 | 583 | PHE0003270_3489 | 1 | soy-CGPG1191-like1 |
| 1325 | 584 | PHE0003277_3496 | 1 | soy-CGPG1430-like1 |
| 1326 | 585 | PHE0003352_3571 | 1 | Corn ethylene receptor 1 |
| 1327 | 586 | PHE0003366_3589 | 1 | transaldolase |
| 1328 | 587 | PHE0003370_3593 | 4 | Circulin B |
| 1329 | 588 | PHE0003526_3772 | 1 | soy G2567 like2 |
| 1330 | 589 | PHE0003581_3827 | 1 | rice G2603 like2 |
| 1331 | 590 | PHE0003681_3942 | 4 | rice G1792-like 3 |
| 1332 | 591 | PHE0003683_3944 | 16 | soy SUC1-like 1 |
| 1333 | 592 | PHE0003683_3945 | 18 | soy SUC1-like 1 |
| 1334 | 593 | PHE0003815_4288 | 4 | Synechocystis NblS-like |
| 1335 | 594 | PHE0003986_4573 | 4 | corn AfMONFEED001223 putative stearoyl- |
| | | | | acyl-carrier protein desaturase |
| 1336 | 595 | PHE0003989_4575 | 4 | corn AfMONFEED000878 remorin-like |
| | | | | protein |
| 1337 | 596 | PHE0003990_4576 | 4 | corn AfMONFEED001231 unknown protein |
| 1338 | 597 | PHE0003995_4581 | 4 | corn AfMONFEED001033 putative succinate |
| | | | | dehydrogenase |
| 1339 | 598 | PHE0004022_4657 | 4 | Arabidopsis Alfin-like |
| 1340 | 599 | PHE0000667_770 | 1 | chimeric glycerol-3-phosphate acyltransferase |
| 1341 | 600 | PHE0000784_857 | 1 | Arabidopsis agl11 delta MADS-box |
| 1342 | 601 | PHE0000880_963 | 1 | Nostoc sp. PCC7120 Hik33 |
| 1343 | 602 | PHE0000883_966 | 1 | Synechocystis sp. 6803 Rer1 |
| 1344 | 603 | PHE0001063_1153 | 1 | yeast SNF4-Z72637 |
| 1345 | 604 | PHE0001065_1155 | 1 | yeast SIP2-Z72730 |
| 1346 | 605 | PHE0001069_1159 | 1 | Arabidopsis bZIP domain transcription factor |
| 1347 | 606 | PHE0001076_1166 | 1 | Arabidopsis NAC domain transcription factor |
| 1348 | 607 | PHE0001080_1170 | 1 | Arabidopsis AP2 domain transcription factor |
| 1349 | 608 | PHE0001087_1177 | 1 | Arabidopsis homeodomain transcription |
| | | | | factor |
| 1350 | 609 | PHE0001094_1184 | 1 | Arabidopsis zinc finger protein |
| 1351 | 610 | PHE0001100_1190 | 1 | Arabidopsis bZIP domain transcription factor |
| 1352 | 611 | PHE0001112_1202 | 1 | yeast alanine aminotransferase 2-CAA88665 |
| 1353 | 612 | PHE0001131_1221 | 5 | yeast acetohydroxyacid reductoisomerase- |
| | | | | AAB67753 |
| 1354 | 613 | PHE0001143_1233 | 1 | corn putative isoprenylated protein |
| 1355 | 614 | PHE0001149_1239 | 1 | corn HvB12D homolog |
| 1356 | 615 | PHE0001169_1259 | 1 | Arabidopsis TFL2-AF387639 |
| 1357 | 616 | PHE0001176_1266 | 5 | Synechocystis sp. PCC 6803 heme |
| | | | | oxygenase-1651897 |
| 1358 | 617 | PHE0001177_1267 | 5 | Nostoc sp. PCC 7120 heme oxygenase- |
| | | | | 17132210 |
| 1359 | 618 | PHE0001184_1274 | 1 | rice SHY2-like 1-BAA92982 |
| 1360 | 619 | PHE0001256_1347 | 1 | Arabidopsis protease HhoA precursor |
| 1361 | 620 | PHE0001264_1355 | 1 | Arabidopsis hypothetical protein |
| 1362 | 621 | PHE0001265_1356 | 1 | Arabidopsis hypothetical protein |
| | | | | [NM_105159] |
| 1363 | 622 | PHE0001268_1359 | 1 | Arabidopsis hypothetical protein |
| 1364 | 623 | PHE0001270_1361 | 1 | yeast ero1-CAA90553 |
| 1365 | 624 | PHE0001311_1401 | 1 | Arabidopsis expressed protein [NM_126228] |
| 1366 | 625 | PHE0001322_1412 | 1 | Arabidopsis expressed protein |
| 1367 | 626 | PHE0001519_1610 | 1 | rice WD domain protein |
| 1368 | 627 | PHE0001523_1614 | 1 | rice LPE1-like permase 1-BAB61205 |
| 1369 | 628 | PHE0001527_1618 | 1 | Nostoc sp. PCC 7120 GDH |
| 1370 | 629 | PHE0001528_1619 | 1 | Nostoc punctiforme GDH |
| 1371 | 630 | PHE0001533_1624 | 1 | Streptomyces coelicolor GDH |
| 1372 | 631 | PHE0001541_1632 | 1 | rice glutamate dehydrogenase-15787849 |
| 1373 | 632 | PHE0001567_1666 | 1 | Bacillus halodurans asparagine synthase- |
| | | | | 10174030 |
| 1374 | 633 | PHE0001568_1667 | 4 | Corynebacterium glutamicum Asparagine |
| | | | | synthase-19551250 |
| 1375 | 634 | PHE0001611_1722 | 1 | rice translation initiation factor 3 delta subunit |
| | | | | like sequence |
| 1376 | 635 | PHE0001632_1743 | 1 | arabidopsis magnesium/proton exchanger |
| | | | | AtMHX |
| 1377 | 636 | PHE0002029_2140 | 1 | Corn Axi 1 |
| 1378 | 637 | PHE0002078_2188 | 1 | yeast sec61 |
| 1379 | 638 | PHE0002084_2194 | 1 | Corn cyclic nucleotide and calmodulin- |
| | | | | regulated ion channel |
| 1380 | 639 | PHE0002089_2199 | 1 | Agrobacterium tumefaciens hypothetical |
| | | | | protein |
| 1381 | 640 | PHE0002090_2200 | 1 | Nostoc sp. PCC 7120 stress induced |
| | | | | hydrophobic peptide |
| 1382 | 641 | PHE0002151_2259 | 1 | Corn Kaurene Synthase |
| 1383 | 642 | PHE0002194_2301 | 1 | maize Betaine-aldehyde dehydrogenase like 1 |
| | | | | sequence |
| 1384 | 643 | PHE0002483_2583 | 1 | maize heat shock TF like sequence |
| 1385 | 644 | PHE0002494_2594 | 1 | Arabidopsis ascorbate oxidase |
| 1386 | 645 | PHE0002509_2609 | 1 | cORN Hsp20/alpha crystallin family” |
| 1387 | 646 | PHE0002523_2623 | 1 | Corn putative thioredoxin protein |
| 1388 | 647 | PHE0002527_2627 | 1 | Hypothetical Nostoc protein similar to cobW |
| 1389 | 648 | PHE0002546_2646 | 1 | Corn dual-specificity protein |
| | | | | phosphatase-like protein |
| 1390 | 649 | PHE0002557_2657 | 1 | soy G1792-like 10 |
| 1391 | 650 | PHE0002566_2665 | 1 | Ribosomal protein L39 |
| 1392 | 651 | PHE0002614_2713 | 1 | Brassica napus sigA binding protein 2 |
| 1393 | 652 | PHE0002648_2767 | 1 | soy AGL21-like 2 |
| 1394 | 653 | PHE0002652_2771 | 1 | Corn GDP-mannose pyrophosphorylase A |
| 1395 | 654 | PHE0002791_2926 | 1 | rice CYP72A5 like 2 sequence |
| 1396 | 655 | PHE0002849_2984 | 1 | Oryza Sativa Putative Glucosyl Transferase |
| 1397 | 656 | PHE0002855_2990 | 1 | Synechocystis sp unknown protein wi/ ABC1 |
| | | | | PFAM domain (putative novel chaperonin) |
| 1398 | 657 | PHE0002856_2991 | 1 | Nostoc sp unknown protein wi/ ABC1 PFAM |
| | | | | domain(putative novel chaperonin) |
| 1399 | 658 | PHE0002886_3021 | 1 | Brassica dsPTP 1 |
| 1400 | 659 | PHE0002888_3023 | 1 | soy dsPTP 2 |
| 1401 | 660 | PHE0002893_3028 | 1 | Corn dsPTP 3 |
| 1402 | 661 | PHE0002901_3036 | 1 | Yeast GAT2 GATA Zinc Finger TF |
| 1403 | 662 | PHE0002906_3041 | 1 | Zea Mays Zinc Finger Transcription Factor |
| | | | | IV |
| 1404 | 663 | PHE0002910_3045 | 1 | Yeast IKS1 |
| 1405 | 664 | PHE0002923_3058 | 1 | Yeast GGA1 protein (mediator of protein |
| | | | | traficking between Trans golgi network and |
| | | | | vacoule) |
| 1406 | 665 | PHE0002928_3065 | 1 | Yeast Zinc Finger protein (DNA damage |
| | | | | responsive repressor of PHR1) |
| 1407 | 666 | PHE0002941_3091 | 1 | Yeast SET6p putative Transcription Factor |
| 1408 | 667 | PHE0002947_3097 | 1 | Zea Mays Sugar & other (polyamine) |
| | | | | transporter like protein |
| 1409 | 668 | PHE0002948_3098 | 1 | Yeast Xylulokinase |
| 1410 | 669 | PHE0002987_3137 | 1 | Yeast oxidoreductase of unknown function |
| | | | | (PFAM NAD-binding Rossmann fold & C- |
| | | | | terminal alpha/beta domain) |
| 1411 | 670 | PHE0003017_3167 | 1 | Yeast Transketolase 2 |
| 1412 | 671 | PHE0003018_3168 | 1 | Yeast Ubiquitin polyprotein |
| 1413 | 672 | PHE0003023_3173 | 1 | Yeast unknown protein (uncharacterized |
| | | | | protein family) |
| 1414 | 673 | PHE0003025_3175 | 1 | Yeast Protein of unknown function, (similar |
| | | | | to mouse MPV17 a putative integral |
| | | | | membrane peroxisomal protein) |
| 1415 | 674 | PHE0003026_3176 | 1 | Yeast Protein of unknown function, putative |
| | | | | paralog of Ecm4p, a cell wall biogenesis |
| | | | | protein |
| 1416 | 675 | PHE0003027_3177 | 1 | Yeast Protein of unknown function (Pfam |
| | | | | Domain YjeF-related protein N-terminus) |
| 1417 | 676 | PHE0003028_3178 | 1 | Yeast Potential alpha-ketoisocaproate |
| | | | | reductase |
| 1418 | 677 | PHE0003030_3180 | 1 | Yeast Yapsin 6, GPI-anchored aspartyl |
| | | | | protease |
| 1419 | 678 | PHE0003062_3212 | 1 | soy G571 |
| 1420 | 679 | PHE0003088_3240 | 1 | Arabidopsis AtPK1 |
| 1421 | 680 | PHE0003089_3237 | 1 | Arabidopsis AtPK19b |
| 1422 | 681 | PHE0003090_3238 | 1 | soy S6K1 |
| 1423 | 682 | PHE0003091_3239 | 1 | corn S6K1 |
| 1424 | 683 | PHE0003102_3244 | 6 | Clostridium acetobutylicum fructokinase |
| 1425 | 684 | PHE0003102_3579 | 16 | Clostridium acetobutylicum fructokinase |
| 1426 | 685 | PHE0003121_3267 | 1 | Arabidopsis cyt P450 like |
| 1427 | 686 | PHE0003130_3276 | 1 | Arabidopsis glucosyltransferase-like |
| 1428 | 687 | PHE0003134_3280 | 1 | Arabidopsis microtubule-associated protein |
| | | | | EB1-like protein like |
| 1429 | 688 | PHE0003194_3393 | 1 | corn seven-in-absentia 2 |
| 1430 | 689 | PHE0003195_3394 | 1 | soy seven-in-absentia 1 |
| 1431 | 690 | PHE0003199_3398 | 1 | rice seven-in-absentia 2 |
| 1432 | 691 | PHE0003201_3400 | 1 | soy seven-in-absentia 4 |
| 1433 | 692 | PHE0003208_3410 | 7 | soy triose phosphate translocator 1 |
| 1434 | 693 | PHE0003213_3419 | 1 | yeast TAT2 |
| 1435 | 694 | PHE0003214_3420 | 1 | yeast CUP1a |
| 1436 | 695 | PHE0003222_3428 | 1 | Corynebacterium glutamicum glutamate 5- |
| | | | | kinase |
| 1437 | 696 | PHE0003238_3454 | 1 | Arabidopsis fructose-bisphosphate aldolase |
| 1438 | 697 | PHE0003242_3458 | 1 | Arabidopsis ABC transporter |
| 1439 | 698 | PHE0003251_3468 | 1 | Arabidopsis unknown protein |
| 1440 | 699 | PHE0003260_3477 | 1 | soy-CGPG1517-like1 |
| 1441 | 700 | PHE0003272_3491 | 1 | Arabidopsis hypothetical protein |
| 1442 | 701 | PHE0003275_3494 | 1 | Brassica-CGPG1391-like1 |
| 1443 | 702 | PHE0003302_3523 | 1 | Arabidopsis hypothetical protein |
| 1444 | 703 | PHE0003308_3527 | 1 | Arabidopsis Yippee putative zinc-binding |
| | | | | protein |
| 1445 | 704 | PHE0003315_3534 | 1 | Arabidopsis unknown protein |
| 1446 | 705 | PHE0003320_3539 | 1 | Arabidopsis PDK1 like 1 |
| 1447 | 706 | PHE0003344_3562 | 1 | corn ADC like-1 |
| 1448 | 707 | PHE0003347_3565 | 1 | Arabidopsis GCN5 |
| 1449 | 708 | PHE0003354_3573 | 1 | corn G571 long splice form A196P, A197P |
| 1450 | 709 | PHE0003380_3603 | 1 | putative laccase |
| 1451 | 710 | PHE0003388_3611 | 4 | Caffeic acid 3-O-methyltransferase |
| 1452 | 711 | PHE0003395_3618 | 4 | hypothetical protein4 |
| 1453 | 712 | PHE0003397_3620 | 1 | hypothetical protein5 |
| 1454 | 713 | PHE0003403_3626 | 16 | potato twin LOV protein |
| 1455 | 714 | PHE0003411_3634 | 1 | Putative NAM protein |
| 1456 | 715 | PHE0003414_3654 | 19 | Streptococcus mutans gtfA (dicot codon |
| | | | | modified) |
| 1457 | 716 | PHE0003415_3655 | 18 | Synechococcus sp. PCC 7942 IctB with |
| | | | | RuBisCO small subunit 1b CTP |
| 1458 | 717 | PHE0003431_3639 | 4 | Streptomyces coelicolor trehalose synthase 1 |
| 1459 | 718 | PHE0003434_3643 | 1 | corn dehalogenase-phosphatase 2 |
| 1460 | 719 | PHE0003436_3645 | 1 | Nostoc sp. PCC 7120 dehalogenase- |
| | | | | phosphatase |
| 1461 | 720 | PHE0003437_3646 | 1 | yeast dehalogenase-phosphatase |
| 1462 | 721 | PHE0003447_3678 | 1 | soy DUF296 |
| 1463 | 722 | PHE0003483_3729 | 1 | soy G2035 like1 |
| 1464 | 723 | PHE0003519_3765 | 1 | corn G2505 like2 |
| 1465 | 724 | PHE0003582_3828 | 1 | soy G2999 like1 |
| 1466 | 725 | PHE0003586_3832 | 1 | Brassica G2763 like1 |
| 1467 | 726 | PHE0003588_3834 | 1 | soy G2776 like1 |
| 1468 | 727 | PHE0003598_3844 | 1 | soy G2981 like1 |
| 1469 | 728 | PHE0003606_3852 | 1 | soy G2898 like1 |
| 1470 | 729 | PHE0003613_3860 | 1 | corn GAD1-1 |
| 1471 | 730 | PHE0003617_3866 | 1 | Brassica G2839 like1 |
| 1472 | 731 | PHE0003632_3890 | 1 | rice G2982 like 1 |
| 1473 | 732 | PHE0003636_3894 | 1 | soy G2992 like1 |
| 1474 | 733 | PHE0003676_3934 | 1 | soy G1052 like2 |
| 1475 | 734 | PHE0003682_3943 | 4 | wheat wpk4 |
| 1476 | 735 | PHE0003788_4122 | 4 | corn NAC1-like 1 |
| 1477 | 736 | PHE0003899_4284 | 17 | CGPG2101 Arabidopsis PHD-finger protein |
| 1478 | 737 | PHE0003948_4528 | 17 | Arabidopsis CGPG3676 |
| 1479 | 738 | PHE0003981_4568 | 4 | corn AfMONFEED000388 pyruvate kinase |
| 1480 | 739 | PHE0003984_4571 | 4 | corn AfMONFEED000668 unknown protein |
| 1481 | 740 | PHE0003998_4584 | 4 | corn AfMONFEED000499 putative indole-3- |
| | | | | acetic acid-regulated protein |
| 1482 | 741 | PHE0004008_4594 | 4 | corn AtMONFEED000474 serine protease- |
| | | | | like protein |

Selection Methods for Transgenic Plants with Enhanced Agronomic Trait

Within a population of transgenic plants regenerated from plant cells transformed with the recombinant DNA many plants that survive to fertile transgenic plants that produce seeds and progeny plants will not exhibit an enhanced agronomic trait. Selection from the population is necessary to identify one or more transgenic plant cells that can provide plants with the enhanced trait. Transgenic plants having enhanced traits are selected from populations of plants regenerated or derived from plant cells transformed as described herein by evaluating the plants in a variety of assays to detect an enhanced trait, e.g. enhanced water use efficiency, enhanced cold tolerance, increased yield, enhanced nitrogen use efficiency, enhanced seed protein and enhanced seed oil. These assays also may take many forms including, but not limited to, direct screening for the trait in a greenhouse or field trial or by screening for a surrogate trait. Such analyses can be directed to detecting changes in the chemical composition, biomass, physiological properties, morphology of the plant. Changes in chemical compositions such as nutritional composition of grain can be detected by analysis of the seed composition and content of protein, free amino acids, oil, free fatty acids, starch or tocopherols. Changes in biomass characteristics can be made on greenhouse or field grown plants and can include plant height, stem diameter, root and shoot dry weights; and, for corn plants, ear length and diameter. Changes in physiological properties can be identified by evaluating responses to stress conditions, for example assays using imposed stress conditions such as water deficit, nitrogen deficiency, cold growing conditions, pathogen or insect attack or light deficiency, or increased plant density. Changes in morphology can be measured by visual observation of tendency of a transformed plant with an enhanced agronomic trait to also appear to be a normal plant as compared to changes toward bushy, taller, thicker, narrower leaves, striped leaves, knotted trait, chlorosis, albino, anthocyanin production, or altered tassels, ears or roots. Other selection properties include days to pollen shed, days to silking, leaf extension rate, chlorophyll content, leaf temperature, stand, seedling vigor, internode length, plant height, leaf number, leaf area, tillering, brace roots, stay green, stalk lodging, root lodging, plant health, barreness/prolificacy, green snap, and pest resistance. In addition, phenotypic characteristics of harvested grain may be evaluated, including number of kernels per row on the ear, number of rows of kernels on the ear, kernel abortion, kernel weight, kernel size, kernel density and physical grain quality. Although the plant cells and methods of this invention can be applied to any plant cell, plant, seed or pollen, e.g. any fruit, vegetable, grass, tree or ornamental plant, the various aspects of the invention are preferably applied to corn, soybean, cotton, canola, alfalfa, wheat and rice plants. In many cases the invention is applied to corn plants that are inherently resistant to disease from the Mal de Rio Cuarto virus or the Puccina sorghi fungus or both.

The following examples are included to demonstrate aspects of the invention, those of skill in the art should, in light of the present disclosure, appreciate that many changes can be made in the specific aspects which are disclosed and still obtain a like or similar results without departing from the spirit and scope of the invention.

### EXAMPLE 1

### Plant Expression Constructs

### A. Plant Expression Constructs for Corn Transformation

This example illustrates the construction of plasmids for transferring recombinant DNA into plant cells which can be regenerated into transgenic plants of this invention.

A base plant transformation vector pMON65154, as set forth in SEQ ID NO: 52768 was fabricated for use in preparing recombinant DNA for transformation into corn tissue using GATEWAY™ Destination plant expression vector systems (available from Invitrogen Life Technologies, Carlsbad, Calif.). With reference to the elements described in Table 3 below, pMON65154 comprises a selectable marker expression cassette and a template recombinant DNA expression cassette. The marker expression cassette comprises a CaMV 35S promoter operably linked to a gene encoding neomycin phosphotransferase II (nptII) followed by a 3′ region of an Agrobacterium tumefaciens nopaline synthase gene (nos). The template recombinant DNA expression cassette is positioned tail to tail with the marker expression cassette. The template recombinant DNA expression cassette comprises 5′ regulatory DNA including a rice actin 1 promoter, exon and intron, followed by a GATEWAY™ insertion site for recombinant DNA, followed by a 3′ region of a potato proteinase inhibitor II (pinII) gene. Once recombinant DNA has been inserted into the insertion site, the plasmid is useful for plant transformation, e.g. by microprojectile bombardment.

TABLE 3

| FUNCTION | ELEMENT | REFERENCE |
|---|---|---|
| Plant gene of interest | Rice actin 1 promoter | U.S. Pat. No. 5,641,876 |
| expression cassette | Rice actin 1 exon 1, intron 1 | U.S. Pat. No. 5,641,876 |
| | enhancer | |
| Gene of interest | AttR1 | GATEWAY ™ Cloning Technology |
| insertion site | | Instruction Manual |
| | CmR gene | GATEWAY ™ Cloning Technology |
| | | Instruction Manual |
| | ccdA, ccdB genes | GATEWAY ™ Cloning Technology |
| | | Instruction Manual |
| | attR2 | GATEWAY ™ Cloning Technology |
| | | Instruction Manual |
| Plant gene of interest | Potato pinII 3′ region | An et al. (1989) Plant Cell 1: 115-122 |
| expression cassette | | |
| Plant selectable | CaMV 35S promoter | U.S. Pat. No. 5,858,742 |
| marker expression | nptII selectable marker | U.S. Pat. No. 5,858,742 |
| cassette | nos 3′ region | U.S. Pat. No. 5,858,742 |
| Maintenance in E. coli | ColE1 origin of replication | |
| | F1 origin of replication | |
| | Bla ampicillin resistance | |

A similar base vector plasmid pMON72472 (SEQ ID NO: 52769) was constructed for use in Agrobacterium-mediated methods of plant transformation similar to pMON65154 except (a) the 5′ regulatory DNA in the template recombinant DNA expression cassette was a rice actin promoter and a rice actin intron, (b) left and right T-DNA border sequences from Agrobacterium are added with the right border sequence is located 5′ to the rice actin 1 promoter and the left border sequence is located 3′ to the 35S promoter and (c) DNA is added to facilitate replication of the plasmid in both E. coli and Agrobacterium tumefaciens. The DNA added to the plasmid outside of the T-DNA border sequences includes an oriV wide host range origin of DNA replication functional in Agrobacterium, a pBR322 origin of replication functional in E. coli, and a spectinomycin/streptomycin resistance gene for selection in both E. coli and Agrobacterium.

Other base vectors similar to those described above were also constructed as listed in Table 4. See Table 4 for a summary of base vector plasmids and base vector ID's which are referenced in Table 1. Also see Table 5 for a summary of regulatory elements used in the gene expression cassette for these base vectors and SEQ ID NOs for elements.

TABLE 4

| Base Vector ID | |
|---|---|
| | Base Vector |
| | for Corn |
| 1 | pMON72472 |
| 2 | pMON65154 |
| 3 | pMON84109 |
| 4 | pMON82060 |
| 5 | pMON74430 |
| 6 | pMON84107 |
| 7 | pMON81244 |
| 8 | pMON76274 |
| 9 | pMON74575 |
| 10 | pMON92667 |
| 11 | pMON84108 |
| 12 | pMON74582 |
| 13 | pMON74579 |
| 14 | pMON74577 |
| | Base Vector |
| | for Soybean |
| 15 | pMON74552 |
| 16 | pMON74532 |
| 17 | pMON82053 |
| 18 | pMON74537 |
| 19 | pMON74536 |
| 20 | pMON74548 |

TABLE 5

| | | SEQ | | SEQ | | SEQ | transit | SEQ | | SEQ |
|---|---|---|---|---|---|---|---|---|---|---|
| Vector | promoter | ID NO | leader | ID NO | intron | ID NO | peptide | ID NO | terminator | ID NO |
| pMON65154 | P-Os.Act1-1:4:27 | 52789 | L-Os.Act1-1:1:3 | 52777 | I-Os.Act1-1:1:40 | 52772 | NONE | / | T-St.Pis4-1:4:3 | 52801 |
| pMON72472 | P-Os.Act1-1:1:8 | 52788 | L-Os.Act1-1:1:5 | 52778 | I-Os.Act1-1:1:3 | 52771 | NONE | / | T-St.Pis4-1:4:1 | 52800 |
| pMON74577 | P-Hv.Per1-1:1:7 | 52787 | L-Hv.Per1-1:1:1 | 52776 | I-Zm.DnaK-1:1:1 | 52773 | NONE | / | T-St.Pis4-1:4:1 | 52800 |
| pMON74579 | P-Zm.NAS2- | 52793 | L-Zm.NAS2- | 52781 | I-Zm.DnaK-1:1:1 | 52773 | NONE | / | T-St.Pis4-1:4:1 | 52800 |
| | 1:1:1 | | 1:1:1 | | | | | | | |
| pMON74582 | P-Zm.PPDK- | 52795 | L-Zm.PPDK- | 52782 | I-Zm.DnaK-1:1:1 | 52773 | NONE | / | T-St.Pis4-1:4:1 | 52800 |
| | 1:1:12 | | 1:1:2 | | | | | | | |
| pMON76274 | P-Os.GT1-1:1:10 | 52790 | L-Os.GT1-1:1:3 | 52779 | I-Zm.DnaK-1:1:1 | 52773 | NONE | / | T-St.Pis4-1:4:1 | 52800 |
| pMON81244 | P-Zm.PPDK- | 52794 | L-Zm.PPDK- | 52782 | I-Zm.DnaK-1:1:1 | 52773 | NONE | / | T-St.Pis4-1:4:3 | 52801 |
| | 1:1:10 | | 1:1:2 | | | | | | | |
| pMON82060 | P-Os.Act1-1:1:8 | 52788 | L-Os.Act1-1:1:5 | 52778 | I-Os.Act1-1:1:3 | 52771 | NONE | / | T-St.Pis4-1:4:1 | 52800 |
| pMON84109 | P-Os.Act1-1:1:8 | 52788 | L-Os.Act1-1:1:5 | 52778 | I-Os.Act1-1:1:3 | 52771 | TS-At.ShkG- | 52799 | T-St.Pis4-1:4:1 | 52800 |
| | | | | | | | CTP2-1:1:1 | | | |
| pMON74430 | P-Os.Act1-1:1:8 | 52788 | L-Os.Act1-1:1:5 | 52778 | I-Os.Act1-1:1:3 | 52771 | TS-At.ShkG- | 52799 | T-St.Pis4-1:4:1 | 52800 |
| | | | | | | | CTP2-1:1:1 | | | |
| pMON84107 | P-Os.GT1-1:1:18 | 52791 | NONE | | I-Zm.DnaK-1:1:1 | 52773 | NONE | / | T-St.Pis4-1:4:1 | 52800 |
| pMON74575 | P-Zm.FDA-1:1:5 | 52792 | L-Zm.FDA-1:1:1 | 52780 | I-Zm.DnaK-1:1:1 | 52773 | NONE | / | T-St.Pis4-1:4:1 | 52800 |
| pMON92667 | P-Zm.SzeinC1- | 52797 | L-Zm.SceinC1- | 52783 | I-Zm.DnaK-1:1:1 | 52773 | NONE | / | T-St.Pis4-1:4:1 | 52800 |
| | 1:1:1 | | 1:1:1 | | | | | | | |
| pMON74532 | P-CaMV.35S- | 52786 | NONE | / | NONE | / | NONE | | T-Gb.E6- | 52798 |
| | enh- | | | | | | | | 3b:1:1 | |
| | 1:1:11 | | | | | | | | | |
| pMON74552 | P-CaMV.35S- | 52786 | NONE | / | NONE | / | TS-At.ShkG- | 52799 | T-Gb.E6- | 52798 |
| | enh- | | | | | | CTP2-1:1:1 | | 3b:1:1 | |
| | 1:1:11 | | | | | | | | | |
| pMON82053 | P-CaMV.35S- | 52786 | NONE | / | NONE | / | NONE | / | T-Gb.E6- | 52798 |
| | enh- | | | | | | | | 3b:1:1 | |
| | 1:1:11 | | | | | | | | | |
| pMON74537 | P-At.RbcS4-1:1:2 | 52784 | L-At.RbcS4-1:1:3 | 52774 | NONE | / | NONE | / | T-Gb.E6- | 52798 |
| | | | | | | | | | 3b:1:1 | |
| pMON74536 | P-Br.Snap2-1:1:1 | 52785 | L-Br.Snap2-1:1:1 | 52775 | NONE | / | NONE | / | T-Gb.E6- | 52798 |
| | | | | | | | | | 3b:1:1 | |
| pMON74548 | P-Gm.Sphas1- | 52796 | L-Gm.Sphas1- | 52802 | NONE | / | NONE | / | T-Gb.E6- | 52798 |
| | 1:1:1 | | 1:1:1 | | | | | | 3b:1:1 | |
| pMON74539 | P-At.RbcS4-1:1:2 | 52784 | L-At.RbcS4-1:1:3 | 52774 | NONE | / | TS-At.ShkG- | 52799 | T-Gb.E6- | 52798 |
| | | | | | | | CTP2-1:1:1 | | | 3b:1:1 |

Plasmids for use in transformation of soybean were also prepared. Elements of an exemplary common expression vector plasmid pMON74532 are shown in Table 6 below.

TABLE 6

| Function | Element | Reference |
|---|---|---|
| Agro transformation | B-ARGtu.right border | Depicker, A. et al (1982) |
| | | Mol Appl Genet 1: 561-573 |
| Antibiotic resistance | CR-Ec.aadA-SPC/STR | |
| Repressor of primers from the ColE1 | CR-Ec.rop | |
| plasmid | | |
| Origin of replication | OR-Ec.oriV-RK2 | |
| Agro transformation | B-ARGtu.left border | Barker, R. F. et al (1983) |
| | | Plant Mol Biol 2: 335-350 |
| Plant selectable marker expression | Promoter with intron and | McDowell et al. (1996) |
| cassette | 5′UTR of Arabidopsis act 7 | Plant Physiol. 111: 699-711. |
| | gene (AtAct7) | |
| | 5′ UTR of Arabidopsis act 7 | |
| | gene | |
| | Intron in 5′UTR of AtAct7 | |
| | Transit peptide region of | Klee, H. J. et al (1987) |
| | Arabidopsis EPSPS | MGG 210: 437-442 |
| | Synthetic CP4 coding region | |
| | with dicot preferred codon | |
| | usage | |
| | A 3′ UTR of the nopaline | U.S. Pat. No. 5,858,742 |
| | synthase gene of | |
| | Agrobacterium tumefaciens | |
| | Ti plasmid | |
| Plant gene of interest expression | Promoter for 35S RNA from | U.S. Pat. No. 5,322,938 |
| cassette | CaMV containing a | |
| | duplication of the −90 to −350 | |
| | region | |
| | Gene of interest insertion site | |
| | Cotton E6 3′ end | GenBank accession |
| | | U30508 |

Primers for PCR amplification of protein coding nucleotides of recombinant DNA were designed at or near the start and stop codons of the coding sequence, in order to eliminate most of the 5′ and 3′ untranslated regions. Each recombinant DNA coding for a protein identified in Table 1 was amplified by PCR prior to insertion into the insertion site of one of the base vectors as referenced in Table 1.

### EXAMPLE 2

### Corn Transformation

This example illustrates plant cell transformation methods useful in producing transgenic corn plant cells, plants, seeds and pollen of this invention and the production and identification of transgenic corn plants and seed with an enhanced trait, i.e. enhanced water use efficiency, enhanced cold tolerance, increased yield, enhanced nitrogen use efficiency, enhanced seed protein and enhanced seed oil. Plasmid vectors were prepared by cloning DNA identified in Table 1 in the identified base vectors for use in corn transformation of corn plant cells to produce transgenic corn plants and progeny plants, seed and pollen.

For Agrobacterium-mediated transformation of corn embryo cells corn plants of a readily transformable line (designated LH59) is grown in the greenhouse and ears harvested when the embryos are 1.5 to 2.0 mm in length. Ears are surface sterilized by spraying or soaking the ears in 80% ethanol, followed by air drying. Immature embryos are isolated from individual kernels on surface sterilized ears. Prior to inoculation of maize cells, Agrobacterium cells are grown overnight at room temperature. Immature maize embryo cells are inoculated with Agrobacterium shortly after excision, and incubated at room temperature with Agrobacterium for 5-20 minutes. Immature embryo plant cells are then co-cultured with Agrobacterium for 1 to 3 days at 23° C. in the dark. Co-cultured embryos are transferred to selection media and cultured for approximately two weeks to allow embryogenic callus to develop. Embryogenic callus is transferred to culture medium containing 100 mg/L paromomycin and subcultured at about two week intervals. Transformed plant cells are recovered 6 to 8 weeks after initiation of selection.

For Agrobacterium-mediated transformation of maize callus immature embryos are cultured for approximately 8-21 days after excision to allow callus to develop. Callus is then incubated for about 30 minutes at room temperature with the Agrobacterium suspension, followed by removal of the liquid by aspiration. The callus and Agrobacterium are co-cultured without selection for 3-6 days followed by selection on paromomycin for approximately 6 weeks, with biweekly transfers to fresh media, and paromomycin resistant callus identified as containing the recombinant DNA in an expression cassette.

For transformation by microprojectile bombardment immature maize embryos are isolated and cultured 3-4 days prior to bombardment. Prior to microprojectile bombardment, a suspension of gold particles is prepared onto which the desired recombinant DNA expression cassettes are precipitated. DNA is introduced into maize cells as described in U.S. Pat. Nos. 5,550,318 and 6,399,861 using the electric discharge particle acceleration gene delivery device. Following microprojectile bombardment, tissue is cultured in the dark at 27 degrees C. Additional transformation methods and materials for making transgenic plants of this invention, for example, various media and recipient target cells, transformation of immature embryos and subsequence regeneration of fertile transgenic plants are disclosed in U.S. Pat. Nos. 6,194,636 and 6,232,526 and U.S. patent application Ser. No. 09/757,089, which are incorporated herein by reference.

To regenerate transgenic corn plants a callus of transgenic plant cells resulting from transformation is placed on media to initiate shoot development in plantlets which are transferred to potting soil for initial growth in a growth chamber at 26 degrees C. followed by a mist bench before transplanting to 5 inch pots where plants are grown to maturity. The regenerated plants are self fertilized and seed is harvested for use in one or more methods to select seed, seedlings or progeny second generation transgenic plants (R2 plants) or hybrids, e.g. by selecting transgenic plants exhibiting an enhanced trait as compared to a control plant.

Transgenic corn plant cells are transformed with recombinant DNA from each of the genes identified in Table 1. Progeny transgenic plants and seed of the transformed plant cells are screened for enhanced water use efficiency, enhanced cold tolerance, increased yield, enhanced nitrogen use efficiency, enhanced seed protein and enhanced seed oil as reported in Example 5.

### EXAMPLE 3

### Soybean Transformation

This example illustrates plant transformation useful in producing the transgenic soybean plants of this invention and the production and identification of transgenic seed for transgenic soybean having enhanced water use efficiency, enhanced cold tolerance, increased yield, enhanced nitrogen use efficiency, enhanced seed protein and enhanced seed oil.

For Agrobacterium mediated transformation, soybean seeds are germinated overnight and the meristem explants excised. The meristems and the explants are placed in a wounding vessel. Soybean explants and induced Agrobacterium cells from a strain containing plasmid DNA with the gene of interest cassette and a plant selectable marker cassette are mixed no later than 14 hours from the time of initiation of seed germination and wounded using sonication. Following wounding, explants are placed in co-culture for 2-5 days at which point they are transferred to selection media for 6-8 weeks to allow selection and growth of transgenic shoots. Trait positive shoots are harvested approximately 6-8 weeks and placed into selective rooting media for 2-3 weeks. Shoots producing roots are transferred to the greenhouse and potted in soil. Shoots that remain healthy on selection, but do not produce roots are transferred to non-selective rooting media for an additional two weeks. Roots from any shoots that produce roots off selection are tested for expression of the plant selectable marker before they are transferred to the greenhouse and potted in soil. Additionally, a DNA construct can be transferred into the genome of a soybean cell by particle bombardment and the cell regenerated into a fertile soybean plant as described in U.S. Pat. No. 5,015,580, herein incorporated by reference.

Transgenic soybean plant cells are transformed with recombinant DNA from each of the genes identified in Table 1. Progeny transgenic plants and seed of the transformed plant cells are screened for enhanced water use efficiency, enhanced cold tolerance, increased yield, enhanced nitrogen use efficiency, enhanced seed protein and enhanced seed oil as reported in Example 5.

### EXAMPLE 4

### Homolog Identification

This example illustrates the identification of homologs of proteins encoded by the DNA identified in Table 1 which is used to provide transgenic seed and plants having enhanced agronomic traits. From the sequence of the homologs, homologous DNA sequence can be identified for preparing additional transgenic seeds and plants of this invention with enhanced agronomic traits.

An “All Protein Database” was constructed of known protein sequences using a proprietary sequence database and the National Center for Biotechnology Information (NCBI) non-redundant amino acid database (nr.aa). For each organism from which a polynucleotide sequence provided herein was obtained, an “Organism Protein Database” was constructed of known protein sequences of the organism; it is a subset of the All Protein Database based oil the NCBI taxonomy ID for the organism.

The All Protein Database was queried using amino acid sequences provided herein as SEQ ID NO: 142 through SEQ ID NO:1482 using NCBI “blastp” program with E-value cutoff of 1e−8. Up to 1000 top hits were kept, and separated by organism names. For each organism other than that of the query sequence, a list was kept for hits from the query organism itself with a more significant E-value than the best hit of the organism. The list contains likely duplicated genes of the polynucleotides provided herein, and is referred to as the Core List. Another list was kept for all the hits from each organism, sorted by E-value, and referred to as the Hit List.

The Organism Protein Database was queried using polypeptide sequences provided herein as SEQ ID NO: 142 through SEQ ID NO:1482 using NCBI “blastp” program with E-value cutoff of 1e−4. Up to 1000 top hits were kept. A BLAST searchable database was constructed based on these hits, and is referred to as “SubDB”. SubDB was queried with each sequence in the Hit List using NCBI “blastp” program with E-value cutoff of 1e−8. The hit with the best E-value was compared with the Core List from the corresponding organism. The hit is deemed a likely ortholog if it belongs to the Core List, otherwise it is deemed not a likely ortholog and there is no further search of sequences in the Hit List for the same organism. Homologs from a large number of distinct organisms were identified and are reported by amino acid sequences of SEQ ID NO: 1483 through SEQ ID NO: 52767. These relationship of proteins of SEQ ID NO: 742 through 166 and homologs of SEQ ID NO: 1482 through 52767 is identified in Table 2. The source organism for each homolog is found in the Sequence Listing.

### EXAMPLE 5

### Selection of Transgenic Plants with Enhanced Agronomic Trait(s)

This example illustrates identification of plant cells of the invention by screening derived plants and seeds for enhanced trait. Transgenic corn seed and plants with recombinant DNA identified in Table 1 were prepared by plant cells transformed with DNA that was stably integrated into the genome of the corn cell. The transgenic seed, plantlets and progeny plants were selected using the methods that measure Transgenic corn plant cells were transformed with recombinant DNA from each of the genes identified in Table 1. Progeny transgenic plants and seed of the transformed plant cells were screened for enhanced water use efficiency, enhanced cold tolerance, increased yield, enhanced nitrogen use efficiency, enhanced seed protein and enhanced seed oil as compared to control plants.

### A. Selection for Enhanced Nitrogen Use Efficiency

The physiological efficacy of transgenic corn plants (tested as hybrids) can be tested for nitrogen use efficiency (NUE) traits in a high-throughput nitrogen (N) selection method. The collected data are compared to the measurements from wildtype controls using a statistical model to determine if the changes are due to the transgene. Raw data were analyzed by SAS software. Results shown herein are the comparison of transgenic plants relative to the wildtype controls.

### (1) Media Preparation for Planting a NUE Protocol

Planting materials used: Metro Mix 200 (vendor: Hummert) Cat. # 10-0325, Scotts Micro Max Nutrients (vendor: Hummert) Cat. # 07-6330, OS 4⅓″×3⅞″ pots (vendor: Hummert) Cat. # 16-1415, OS trays (vendor: Hummert) Cat. # 16-1515, Hoagland's macronutrients solution, Plastic 5″ stakes (vendor: Hummert) yellow Cat. # 49-1569, white Cat. # 49-1505, Labels with numbers indicating material contained in pots. Fill 500 pots to rim with Metro Mix 200 to a weight of ˜140 g/pot. Pots are filled uniformly by using a balancer. Add 0.4 g of Micro Max nutrients to each pot. Stir ingredients with spatula to a depth of 3 inches while preventing material loss.

### (2) Planting a NUE Selection in the Greenhouse

(a) Seed Germination—Each pot is lightly altered twice using reverse osmosis purified water. The first watering is scheduled to occur just before planting; and the second watering, after the seed has been planted in the pot. Ten Seeds of each entry (1 seed per pot) are planted to select eight healthy uniform seedlings. Additional wild type controls are planted for use as border rows. Alternatively, 15 seeds of each entry (1 seed per pot) are planted to select 12 healthy uniform seedlings (this larger number of plantings is used for the second, or confirmation, planting). Place pots on each of the 12 shelves in the Conviron growth chamber for seven days. This is done to allow more uniform germination and early seedling growth. The following growth chamber settings are 25° C./day and 22° C./night, 14 hours light and ten hours dark, humidity ˜80%, and light intensity ˜350 μmol/m²/s (at pot level). Watering is done via capillary matting similar to greenhouse benches with duration of ten minutes three times a day.

(b) Seedling transfer—After seven days, the best eight or 12 seedlings for the first or confirmation pass runs, respectively, are chosen and transferred to greenhouse benches. The pots are spaced eight inches apart (center to center) and are positioned on the benches using the spacing patterns printed on the capillary matting. The Vattex matting creates a 384-position grid, randomizing all range, row combinations. Additional pots of controls are placed along the outside of the experimental block to reduce border effects.

Plants are allowed to grow for 28 days under the low N run or for 23 days under the high N run. The macronutrients are dispensed in the form of a macronutrient solution (see composition below) containing precise amounts of N added (2 mM NH₄NO₃ for limiting N selection and 20 mM NH₄NO₃ for high N selection runs). Each pot is manually dispensed 100 ml of nutrient solution three times a week on alternate days starting at eight and ten days after planting for high N and low N runs, respectively. On the day of nutrient application, two 20 min waterings at 05:00 and 13:00 are skipped. The vattex matting should be changed every third run to avoid N accumulation and buildup of root matter. Table 7 shows the amount of nutrients in the nutrient solution for either the low or high nitrogen selection.

TABLE 7

| | 2 mM NH₄NO₃ | 20 mM NH₄NO₃ |
|---|---|---|
| | (Low Nitrogen | (high |
| | Growth | Nitrogen Growth |
| | Condition, | Condition, |
| | Low N) | High N) |
| Nutrient Stock | mL/L | mL/L |
| 1 M NH₄N0₃ | 2 | 20 |
| 1 M KH₂PO₄ | 0.5 | 0.5 |
| 1 M MgSO₄•7H₂O | 2 | 2 |
| 1 M CaCl₂ | 2.5 | 2.5 |
| 1 M K₂SO₄ | 1 | 1 |

Note:
Adjust pH to 5.6 with HCl or KOH

(c) Harvest Measurements and Data Collection—After 28 days of plant growth for low N runs and 23 days of plant growth for high N runs, the following measurements are taken (phenocodes in parentheses): total shoot fresh mass (g) (SFM) measured by Sartorius electronic balance, V6 leaf chlorophyll measured by Minolta SPAD meter (relative units) (LC), V6 leaf area (cm²) (LA) measured by a Li-Cor leaf area meter, V6 leaf fresh mass (g) (LFM) measured by Sartorius electronic balance, and V6 leaf dry mass (g) (LDM) measured by Sartorius electronic balance. Raw data were analyzed by SAS software. Results shown are the comparison of transgenic plants relative to the wildtype controls.

To take a leaf reading, samples were excised from the V6 leaf. Since chlorophyll meter readings of corn leaves are affected by the part of the leaf and the position of the leaf on the plant that is sampled, SPAD meter readings were done on leaf six of the plants. Three measurements per leaf were taken, of which the first reading was taken from a point one-half the distance between the leaf tip and the collar and halfway from the leaf margin to the midrib while two were taken toward the leaf tip. The measurements were restricted in the area from ½ to ¾ of the total length of the leaf (from the base) with approximately equal spacing between them. The average of the three measurements was taken from the SPAD machine.

Leaf fresh mass is recorded for an excised V6 leaf, the leaf is placed into a paper bag. The paper bags containing the leaves are then placed into a forced air oven at 80° C. for 3 days. After 3 days, the paper bags are removed from the oven and the leaf dry mass measurements are taken.

From the collected data, two derived measurements are made: (1) Leaf chlorophyll area (LCA), which is a product of V6 relative chlorophyll content and its leaf area (relative units). Leaf chlorophyll area=leaf chlorophyll X leaf area. This parameter gives an indication of the spread of chlorophyll over the entire leaf area; (2) specific leaf area (LSA) is calculated as the ratio of V6 leaf area to its dry mass (cm²/g dry mass), a parameter also recognized as a measure of NUE. The data are shown in Table 8.

### Nitrogen Use Field Efficacy Assay

Level I. Transgenic plants provided by the present invention are planted in field without any nitrogen source being applied. Transgenic plants and control plants are grouped by genotype and construct with controls arranged randomly within genotype blocks. Each type of transgenic plants are tested by 3 replications and across 5 locations. Nitrogen levels in the fields are analyzed in early April pre-planting by collecting 30 sample soil cores from 0-24″ and 24 to 48″ soil layer. Soil samples are analyzed for nitrate-nitrogen, phosphorus (P), Potassium (K), organic matter and pH to provide baseline values. P, K and micronutrients are applied based upon soil test recommendations.

Level II. Transgenic plants provided by the present invention are planted in field with three levels of nitrogen (N) fertilizer being applied, i.e. low level (0 N), medium level (80 lb/ac) and high level (180 lb/ac). Liquid 28% or 32% UAN (Urea, Ammonium Nitrogen) are used as the N source and apply by broadcast boom and incorporate with a field cultivator with rear rolling basket in the same direction as intended crop rows. Although there is no N applied to the 0 N treatment the soil should still be disturbed in the same fashion as the treated area. Transgenic plants and control plants are grouped by genotype and construct with controls arranged randomly within genotype blocks. Each type of transgenic plants is tested by 3 replications and across 4 locations. Nitrogen levels in the fields are analyzed in early April pre-planting by collecting 30 sample soil cores from 0-24″ and 24 to 48″ soil layer. Soil samples are analyzed for nitrate-nitrogen, phosphorus (P), Potassium (K), organic matter and pH to provide baseline values. P. K and micronutrients are applied based upon soil test recommendations.

### B. Selection for Increased Yield

Many transgenic plants of this invention exhibit improved yield as compared to a control plant. Improved yield can result from enhanced seed sink potential, i.e. the number and size of endosperm cells or kernels and/or enhanced sink strength, i.e. the rate of starch biosynthesis. Sink potential can be established very early during kernel development, as endosperm cell number and size are determined within the first few days after pollination.

Much of the increase in corn yield of the past several decades has resulted from an increase in planting density. During that period, corn yield has been increasing at a rate of 2.1 bushels/acre/year, but the planting density has increased at a rate of 250 plants/acre/year. A characteristic of modern hybrid corn is the ability of these varieties to be planted at high density. Many studies have shown that a higher than current planting density should result in more biomass production, but current germplasm does not perform. well at these higher densities. One approach to increasing yield is to increase harvest index (HI), the proportion of biomass that is allocated to the kernel compared to total biomass, in high density plantings.

Effective yield selection of enhanced yielding transgenic corn events uses hybrid progeny of the transgenic event over multiple locations with plants grown under optimal production management practices, and maximum pest control. A useful target for improved yield is a 5% to 10% increase in yield as compared to yield produced by plants grown from seed for a control plant. Selection methods may be applied in multiple and diverse geographic locations, for example up to 16 or more locations, over one or more plating seasons, for example at least two planting seasons to statistically distinguish yield improvement from natural environmental effects. It is to plant multiple transgenic plants, positive and negative control plants, and pollinator plants in standard plots, for example 2 row plots, 20 feet long by 5 feet wide with 30 inches distance between rows and a 3 foot alley between ranges. Transgenic events can be grouped by recombinant DNA constructs with groups randomly placed in the field. A pollinator plot of a high quality corn line is planted for every two plots to allow open pollination when using male sterile transgenic events. A useful planting density is about 30,000 plants/acre. High planting density is greater than 30,000 plants/acre, preferably about 40,000 plants/acre, more preferably about 42,000 plants/acre, most preferably about 45,000 plants/acre. Surrogate indicators for yield improvement include source capacity (biomass), source output (sucrose and photosynthesis), sink components (kernel size, ear size, starch in the seed), development (light response, height, density tolerance), maturity, early flowering trait and physiological responses to high density planting, for example at 45,000 plants per acre, for example as illustrated in Table 10 and 11.

TABLE 8

| Timing | Evaluation | Description | comments |
|---|---|---|---|
| V2-3 | Early stand | Can be taken any time after | |
| | | germination and prior to | |
| | | removal of any plants. | |
| Pollen shed | GDU to 50% shed | GDU to 50% plants shedding | |
| | | 50% tassel. | |
| Silking | GDU to 50% silk | GDU to 50% plants showing | |
| | | silks. | |
| Maturity | Plant height | Height from soil surface to | 10 plants per plot - Yield |
| | | flag leaf attachment (inches). | team assistance |
| Maturity | Ear height | Height from soil surface to | 10 plants per plot - Yield |
| | | primary ear attachment node. | team assistance |
| Maturity | Leaves above ear | visual scores: erect, size, | |
| | | rolling | |
| Maturity | Tassel size | Visual scores +/− vs. WT | |
| Pre-Harvest | Final Stand | Final stand count prior to | |
| | | harvest, exclude tillers | |
| Pre-Harvest | Stalk lodging | No. of stalks broken below | |
| | | the primary ear attachment. | |
| | | Exclude leaning tillers | |
| Pre-Harvest | Root lodging | No. of stalks leaning >45° | |
| | | angle from perpendicular. | |
| Pre-Harvest | Stay green | After physiological maturity | |
| | | and when differences among | |
| | | genotypes are evident: Scale | |
| | | 1 (90-100% tissue green)-9 | |
| | | (0-19% tissue green). | |
| Harvest | Grain Yield | Grain yield/plot (Shell | |
| | | weight) | |

TABLE 9

| Timing | Evaluation | Description |
|---|---|---|
| V8-V12 | Chlorophyll | |
| V12-VT | Ear leaf area | |
| V15-15DAP | Chl fluorescence | |
| V15-15DAP | CER | |
| 15-25 DAP | Carbohydrates | sucrose, starch |
| Pre-Harvest | 1st internode diameter | |
| Pre-Harvest | Base 3 internode diameter | |
| Pre-Harvest | Ear internode diameter | |
| Maturity | Ear traits | diameter, length, kernel |
| | | number, kernel weight |

Electron transport rates (ETR) and CO₂ exchange rates (CER): ETR and CER are measured with Li6400LCF (Licor, Lincoln, Nebr.) around V9-R1 stages. Leaf chlorophyll fluorescence is a quick way to monitor the source activity and is reported to be highly correlated with CO₂ assimilation under varies conditions (Photosyn Research, 37: 89-102). The youngest fully expanded leaf or 2 leaves above the ear leaf is measured with actinic light 1500 (with 10% blue light) micromol m⁻² s⁻¹, 28° C., CO₂ levels 450 ppm. Ten plants are measured in each event. There were 2 readings for each plant.

A hand-held chlorophyll meter SPAD-502 (Minolta—Japan) is used to measure the total chlorophyll level on live transgenic plants and the wild type counterparts a. Three trifoliates from each plant are analyzed, and each trifoliate were analyzed three times. Then 9 data points are averaged to obtain the chlorophyll level. The number of analyzed plants of each genotype ranges from 5 to 8.

When selecting for yield improvement a useful statistical measurement approach comprises three components, i.e. modeling spatial autocorrelation of the test field separately for each location, adjusting traits of recombinant DNA events for spatial dependence for each location, and conducting an across location analysis. The first step in modeling spatial autocorrelation is estimating the covariance parameters of the semivariogram. A spherical covariance model is assumed to model the spatial autocorrelation. Because of the size and nature of the trial, it is likely that the spatial autocorrelation may change. Therefore, anisotropy is also assumed along with spherical covariance structure. The following set of equations describes the statistical form of the anisotropic spherical covariance model.

$$C ( h ; θ ) = vI ( h = 0 ) + {σ}^{2} ( 1 - \frac{3}{2} h + \frac{1}{2} {h}^{3} ) I ( h < 1 ) ,$$

where I(•) is the indicator function, h=\sqrt{\dot{x}²+\dot{y}²}, and

\dot{x}=[cos(ρπ/180)(x₁−x₂)−sin(ρπ/180)(y₁−y₂)]/ωₓ

\dot{y}=[sin(ρπ/180)(x₁−x₂)+cos(ρπ/180)(y₁−y₂)]/ω_{y}

where s₁=(x₁, y₁) are the spatial coordinates of one location and s₂=(x₂, y₂) are the spatial coordinates of the second location. There are 5 covariance parameters, θ=(ν, σ², ρ, ωₙ, ωⱼ), where ν is the nugget effect, σ² is the partial sill, ρ is a rotation in degrees clockwise from north, ωₙ is a scaling parameter for the minor axis and ωⱼ is a scaling parameter for the major axis of an anisotropical ellipse of equal covariance. The five covariance parameters that defines the spatial trend will then be estimated by using data from heavily replicated pollinator plots via restricted maximum likelihood approach. In a multi-location field trial, spatial trend are modeled separately for each location.

After obtaining the variance parameters of the model, a variance-covariance structure is generated for the data set to be analyzed. This variance-covariance structure contains spatial information required to adjust yield data for spatial dependence. In this case, a nested model that best represents the treatment and experimental design of the study is used along with the variance-covariance structure to adjust the yield data. During this process the nursery or the seed batch effects can also be modeled and estimated to adjust the yields for any yield parity caused by seed batch differences. After spatially adjusted data from different locations are generated, all adjusted data is combined and analyzed assuming locations as replications. In this analysis, intra and inter-location variances are combined to estimate the standard error of yield from transgenic plants and control plants. Relative mean comparisons are used to indicate statistically significant yield improvements.

### C. Selection for Enhanced Water Use Efficiency (WUE)

Described in this example is a high-throughput method for greenhouse selection of transgenic corn plants to wild type corn plants (tested as inbreds or hybrids) for water use efficiency. This selection process imposes 3 drought/re-water cycles on plants over a total period of 15 days after an initial stress free growth period of 11 days. Each cycle consists of 5 days, with no water being applied for the first four days and a water quenching on the 5th day of the cycle. The primary phenotypes analyzed by the selection method are the changes in plant growth rate as determined by height and biomass during a vegetative drought treatment. The hydration status of the shoot tissues following the drought is also measured. The plant height are measured at three tine points. The first is taken just prior to the onset drought when the plant is 11 days old, which is the shoot initial height (SIH). The plant height is also measured halfway throughout the drought/re-water regimen, on day 18 after planting, to give rise to the shoot mid-drought height (SMH). Upon the completion of the final drought cycle on day 26 after planting, the shoot portion of the plant is harvested and measured for a final height, which is the shoot wilt height (SWH) and also measured for shoot wilted biomass (SWM). The shoot is placed in water at 40 degree Celsius in the dark. Three days later, the shoot is weighted to give rise to the shoot turgid weight (STM). After drying in an oven for four days, the shoots are weighted for shoot dry biomass (SDM). The shoot average height (SAH) is the mean plant height across the 3 height measurements. The procedure described above may be adjusted for +/−˜one day for each step given the situation.

To correct for slight differences between plants, a size corrected growth value is derived from SIH and SWH. This is the Relative Growth Rate (RGR). Relative Growth Rate (RGR) is calculated for each shoot using the formula [RGR %=(SWH−SIH)/((SWH+SIH)/2)*100]. Relative water content (RWC) is a measurement of how much (%) of the plant was water at harvest. Water Content (RwC) is calculated for each shoot using the formula [RWC %=(SWM−SDM)/(STM−SDM)*100]. Fully watered corn plants of this age run around 98% RWC.

### D. Selection for Growth Under Cold Stress

(1) Cold germination assay—Three sets of seeds are used for the assay. The first set consists of positive transgenic events (F1 hybrid) where the genes of the present invention are expressed in the seed. The second seed set is nontransgenic, wild-type negative control made from the same genotype as the transgenic events. The third set consisted of two cold tolerant and one cold sensitive commercial check lines of corn. All seeds are treated with a fungicide “Captan” (MAESTRO® 80DF Fungicide, Arvesta Corporation, San Francisco, Calif., USA). 0.43 mL Captan is applied per 45 g of corn seeds by mixing it well and drying the fungicide prior to the experiment.

Corn kernels are placed embryo side down on blotter paper within an individual cell (8.9×8.9 cm) of a germination tray (54×36 cm). Ten seeds from an event are placed into one cell of the germination tray. Each tray can hold 21 transgenic events and 3 replicates of wildtype (LH244SDms+LH59), which is randomized in a complete block design. For every event there are five replications (five trays). The trays are placed at 9.7 C for 24 days (no light) in a Convrion growth chamber (Conviron Model PGV36, Controlled Environments, Winnipeg, Canada). Two hundred and fifty milliliters of deionized water are added to each germination tray. Germination counts are taken 10th, 11th, 12th, 13th, 14th, 17th, 19th, 21st, and 24th day after start date of the experiment. Seeds are considered germinated if the emerged radical size is 1 cm. From the germination counts germination index is calculated.

The germination index iv calculated as per.

Germination index=(Σ([T+1−nᵢ]*[Pᵢ−Pᵢ₋₁]))/T

Where T is the total number of days for which the germination assay is performed. The number of days after planting is defined by n. “i” indicated the number of times the germination had been counted, including the current day. P is the percentage of seeds germinated during any given rating. Statistical differences are calculated between transgenic events and wild type control. After statistical analysis, the events that show a statistical significance at the p level of less than 0.1 relative to wild-type controls will advance to a secondary cold selection. The secondary cold screen is conducted in the same manner of the primary selection only increasing the number of repetitions to ten. Statistical analysis of the data from the secondary selection is conducted to identify the events that show a statistical significance at the p level of less than 0.05 relative to wild-type controls.

(2) Cold Shock assay—The experimental set-up for the cold shock assay is the same as described in the above cold germination assay except seeds were grown in potted media for the cold shock assay.

The desired numbers of 2.5″ square plastic pots are placed on flats (n=32, 4×8). Pots were filled with Metro Mix 200 soil-less media containing 19:6:12 fertilizer (6 lbs/cubic yard) (Metro Mix, Pots and Flat are obtained from Hummert International, Earth City, Mo.). After planting seeds, pots are placed in a growth chamber set at 23° C., relative humidity of 65% with 12 hour day and night photoperiod (300 uE/m2-min). Planted seeds are watered for 20 minute every other day by sub-irrigation and flats were rotated every third day in a growth chamber for growing corn seedlings.

On the 10^{th} day after planting the transgenic positive and wild-type negative (WT) plants are positioned in flats in an alternating pattern. Chlorophyll fluorescence of plants is measured on the 10^{th} day during the dark period of growth by using a PAM-2000 portable fluorometer as per the manufacturer's instructions (Walz, Germany). After chlorophyll measurements, leaf samples from each event are collected for confirming the expression of genes of the present invention. For expression analysis six V1 leaf tips from each selection are randomly harvested. The flats are moved to a growth chamber set at 5° C. All other conditions Such as humidity, day/night cycle and light intensity are held constant in the growth chamber. The flats are sub-irrigated every day after transfer to the cold temperature. On the 4^{th} day chlorophyll fluorescence is measured. Plants are transferred to normal growth conditions after six days of cold shock treatment and allowed to recover for the next three days. During this recovery period the length of the V3 leaf is measured on the 1^{st} and 3^{rd} days. After two days of recovery V2 leaf damage is determined visually by estimating percent of green V2 leaf.

Statistical differences in V3 leaf growth, V2 leaf necrosis and fluorescence during pre-shock and cold shock can be used for estimation of cold shock damage on corn plants.

(3) Early seedling growth assay—Three sets of seeds are used for the experiment. The first set consists of positive transgenic events (F1 hybrid) where the genes of the present invention are expressed in the seed. The second seed set is nontransgenic, wild-type negative control made from the same genotype as the transgenic events. The third seed set consists of two cold tolerant and two cold sensitive commercial check lines of corn. All seeds are treated with a fungicide “Captan”, (3a,4,7a-tetrahydro-2-[(trichloromethly)thio]-1H-isoindole-1,3-(2H)-dione, Drex Chemical Co. Memphis, Tenn.). Captan (0.43 mL) was applied per 45 g of corn seeds by mixing it well and drying the fungicide prior to the experiment.

Seeds are grown in germination paper for the early seedling growth assay. Three 12″×18″ pieces of germination paper (Anchor Paper #SD7606) are used for each entry in the test (three repetitions per transgenic event). The papers are wetted in a solution of 0.5% KNO₃ and 0.1% Thyram.

For each paper fifteen seeds are placed on the line evenly spaced down the length of the paper. The fifteen seeds are positioned on the paper such that the radical would grow downward, for example longer distance to the paper's edge. The wet paper is rolled up starting from one of the short ends. The paper is rolled evenly and tight enough to hold the seeds in place. The roll is secured into place with two large paper clips, one at the top and one at the bottom. The rolls are incubated in a growth chamber at 23° C. for three days in a randomized complete block design within an appropriate container. The chamber is set for 65% humidity with no light cycle. For the cold stress treatment the rolls are then incubated in a growth chamber at 12° C. for twelve days. The chamber is set for 65% humidity with no light cycle.

After the cold treatment the germination papers are unrolled and the seeds that did not germinate are discarded. The lengths of the radical and coleoptile for each seed are measured through an automated imaging program that automatically collects and processes the images. The imaging program automatically measures the shoot length, root length, and whole seedling length of every individual seedling and then calculates the average of each roll.

After statistical analysis, the events that show a statistical significance at the p level of less than 0.1 relative to wild-type controls will advance to a secondary cold selection. The secondary cold selection is conducted in the same manner of the primary selection only increasing the number of repetitions to five. Statistical analysis of the data from the secondary selection is conducted to identify the events that show a statistical significance at the p level of less than 0.05 relative to wild-type controls.

### 4. Cold Field Efficacy Trial

This example sets forth a cold field efficacy trial to identify gene constructs that confer enhanced cold vigor at germination and early seedling growth under early spring planting field conditions in conventional-till and simulated no-till environments. Seeds are planted into the ground around two weeks before local farmers are beginning to plant corn so that a significant cold stress is exerted onto the crop, named as cold treatment. Seeds also are planted under local optimal planting conditions such that the crop has little or no exposure to cold condition, named as normal treatment. The cold field efficacy trials are carried out in five locations, including Glyndon Minn., Mason Mich., Monmouth Ill., Dayton Iowa, Mystic Conn. At each location, seeds are planted under both cold and normal conditions with 3 repetitions per treatment, 20 kernels per row and single row per plot. Seeds are planted 1.5 to 2 inch deep into soil to avoid muddy conditions. Two temperature monitors are set up at each location to monitor both air and soil temperature daily.

Seed emergence is defined as the point when the growing shoot breaks the soil surface. The number of emerged seedling in each plot is counted everyday from the day the earliest plot begins to emerge until no significant changes in emergence occur. In addition, for each planting date, the latest date when emergence is 0 in all plots is also recorded. Seedling vigor is also rated at V3-V4 stage before the average of corn plant height reaches 10 inches, with 1=excellent early growth, 5=Average growth and 9=poor growth. Days to 50% emergence, maximum percent emergence and seedling vigor are calculated using SAS software for the data within each location or across all locations.

E. Screens for Transgenic Plant Seeds with Increased Protein and/or Oil Levels

This example sets forth a high-throughput selection for identifying plant seeds with improvement in seed composition using the Infratec 1200 series Grain Analyzer, which is a near-infrared transmittance spectrometer used to determine the composition of a bulk seed sample. Near infrared analysis is a non-destructive, high-throughput method that can analyze multiple traits in a single sample scan. An NIR calibration for the analytes of interest is used to predict the values of an unknown sample. The NIR spectrum is obtained for the sample and compared to the calibration using a complex chemometric software package that provides a predicted values as well as information on how well the sample fits in the calibration.

Infratec Model 1221, 1225, or 1227 with transport module by Foss North America is used with cuvette, item # 1000-4033, Foss North America or for small samples with small cell cuvette, Foss standard cuvette modified by Leon Girard Co. Corn and soy check samples of varying composition maintained in check cell cuvettes are supplied by Leon Girard Co. NIT collection software is provided by Maximum Consulting Inc. Software. Calculations are performed automatically by the software. Seed samples are received in packets or containers with barcode labels from the customer. The seed is poured into the cuvettes and analyzed as received.

TABLE 10

| | |
|---|---|
| Typical sample(s): | Whole grain corn and soybean seeds |
| Analytical time to run method: | Less than 0.75 min per sample |
| Total elapsed time per run: | 1.5 minute per sample |
| Typical and minimum sample size: | Corn typical: 50 cc; minimum 30 cc |
| | Soybean typical: 50 cc; |
| | minimum 5 cc |
| Typical analytical range: | Determined in part by the specific |
| | calibration. |
| | Corn - moisture 5-15%, oil 5-20%, |
| | protein 5-30%, starch 50-75%, |
| | and density 1.0-1.3%. |
| | Soybean - moisture 5-15%, |
| | oil 15-25%, |
| | and protein 35-50%. |

### EXAMPLE 6

### Consensus Sequence

This example illustrates the identification of consensus amino acid sequence for the proteins and homologs encoded by DNA that is used to prepare the transgenic seed and plants of this invention having enhanced agronomic traits.

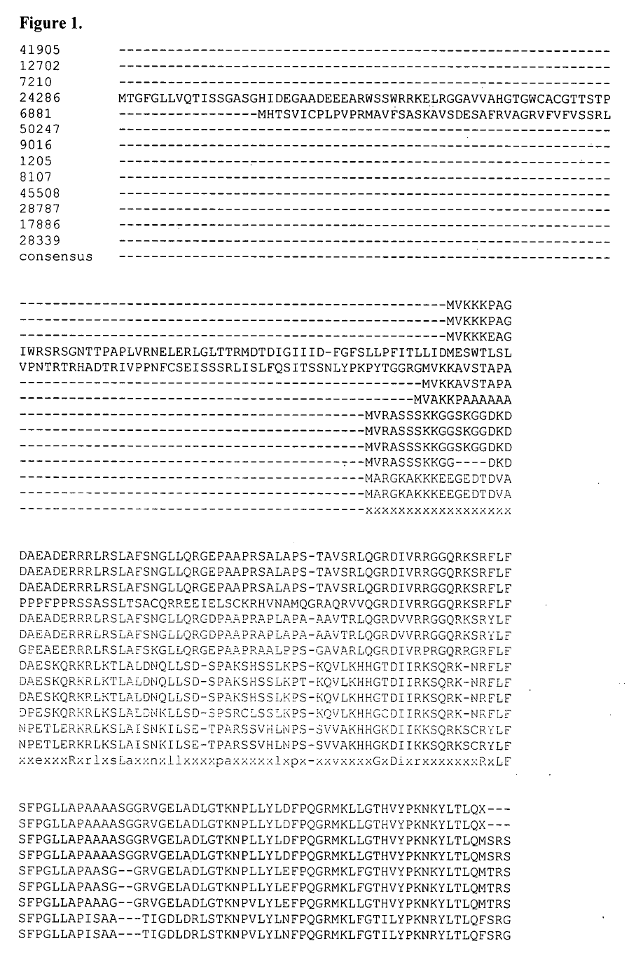

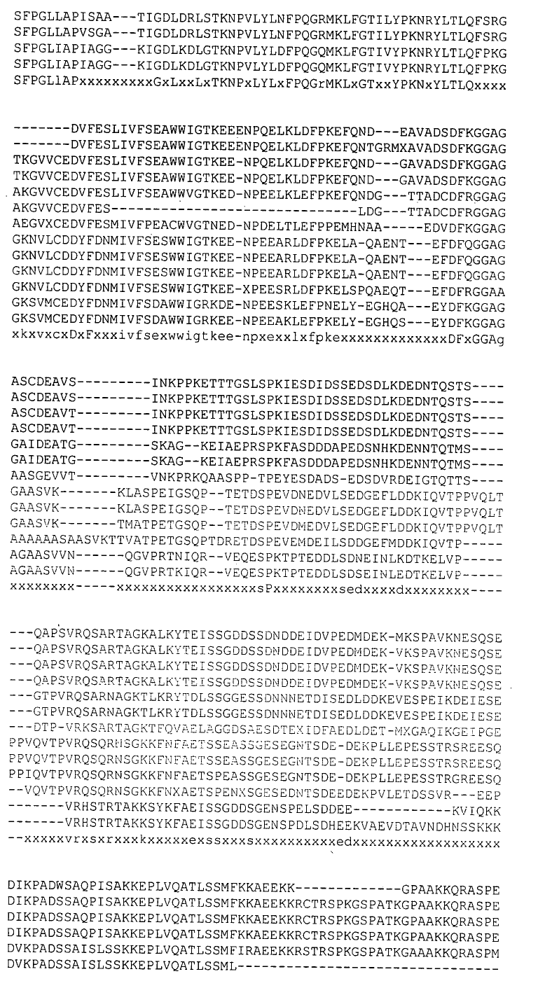

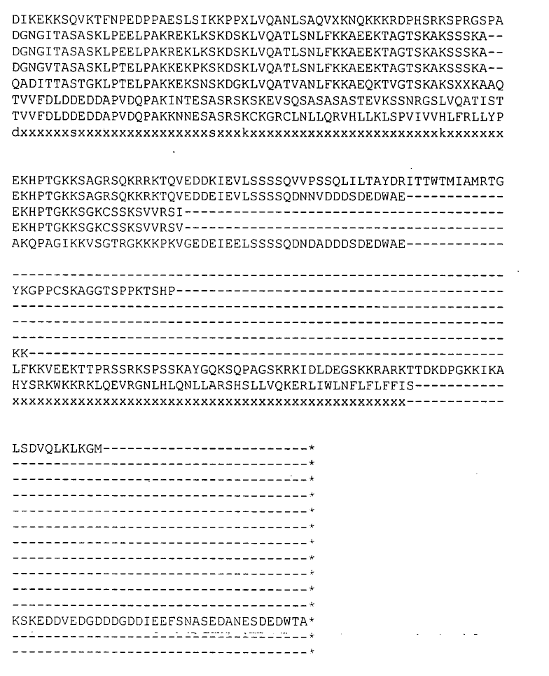

ClustalW program was selected for multiple sequence alignments of the amino acid sequence of SEQ ID NO: 1205 and its 12 homologs. Three major factors affecting the sequence alignments dramatically are (1) protein weight matrices; (2) gap open penalty; (3) gap extension penalty. Protein weight matrices available for ClustalW program include Blosum, Pam and Gonnet series. Those parameters with gap open penalty and gap extension penalty were extensively tested. On the basis of the test results, Blosum weight matrix, gap open penalty of 10 and gap extension penalty of 1 were chosen for multiple sequence alignment. FIG. 2 shows the sequences of SEQ ID NO: 1205, its homologs and the consensus sequence (SEQ ID NO: 52803) at the end. The symbols for consensus sequence are (1) uppercase letters for 100% identity in all positions of multiple sequence alignment output; (2) lowercase letters for >=70% identity; symbol; (3) “X” indicated <70% identity; (4) dashes “−” meaning that gaps were in >=70% sequences.

The consensus amino acid sequence can be used to identify DNA corresponding to the full scope of this invention that is useful in providing transgenic plants, for example corn and soybean plants with enhanced agronomic traits, for example improved nitrogen use efficiency, improved yield, improved water use efficiency and/or improved growth under cold stress, due to the expression in the plants of DNA encoding a protein with amino acid sequence identical to the consensus amino acid sequence.

### EXAMPLE 7

### Identification of Amino Acid Domain by Pfam Analysis

The amino acid sequence of the expressed proteins that were shown to be associated with an enhanced trait were analyzed for Pfam protein family against the current Pfam collection of multiple sequence alignments and hidden Markov models using the HMMER software in the appended computer listing. The Pfam protein families for the proteins of SEQ ID NO: 742 through 1482 are shown in Table 11. The Hidden Markov model databases for the identified patent families are also in the appended computer listing allowing identification of other homologous proteins and their cognate encoding DNA to enable the full breadth of the invention for a person of ordinary skill in the art. Certain proteins are identified by a single Pfam domain and others by multiple Pfam domains. For instance, the protein with amino acids of SEQ ID NO: 817 is characterized by two Pfam domains, i.e. GTP_EFTU, GTP_EFTU_D2 and GTP_EFTU_D3.

TABLE 11

| PEP | | | | | | | |
|---|---|---|---|---|---|---|---|
| SEQ | NUC | | | | | | |
| ID | SEQ | | | | | | |
| NO | ID NO | GENE ID | Pfam domain name | begin | stop | score | E-value |
| 816 | 75 | PHE0000035_61 | bZIP_1 | 15 | 79 | 41.9 | 2.00E−09 |
| 816 | 75 | PHE0000035_61 | bZIP_2 | 15 | 69 | 42.8 | 1.10E−09 |
| 817 | 76 | PHE0000036_62 | GTP_EFTU | 5 | 227 | 359.5 | 4.70E−105 |
| 817 | 76 | PHE0000036_62 | GTP_EFTU_D2 | 248 | 315 | 91.9 | 1.70E−24 |
| 817 | 76 | PHE0000036_62 | GTP_EFTU_D3 | 322 | 430 | 207.6 | 2.60E−59 |
| 818 | 77 | PHE0000130_220 | Jacalin | 185 | 318 | 84.5 | 3.00E−22 |
| 1177 | 436 | PHE0000132_222 | PCI | 63 | 159 | 44.2 | 4.00E−10 |
| 819 | 78 | PHE0000134_224 | LEA_5 | 1 | 113 | 151.2 | 2.40E−42 |
| 820 | 79 | PHE0000135_225 | PP2C | 39 | 281 | 226.2 | 6.60E−65 |
| 821 | 80 | PHE0000136_226 | Hist_deacetyl | 31 | 344 | 579.9 | 2.20E−171 |
| 822 | 81 | PHE0000139_229 | peroxidase | 19 | 227 | 239.9 | 4.80E−69 |
| 823 | 82 | PHE0000141_231 | Thioredoxin | 184 | 295 | −0.9 | 2.00E−05 |
| 824 | 83 | PHE0000142_232 | Ras | 11 | 179 | 280.2 | 3.60E−81 |
| 825 | 84 | PHE0000146_236 | Ras | 13 | 174 | 335 | 1.10E−97 |
| 826 | 85 | PHE0000148_238 | Pkinase | 12 | 267 | 323.2 | 3.90E−94 |
| 826 | 85 | PHE0000148_238 | Pkinase_Tyr | 12 | 265 | 70.9 | 3.70E−18 |
| 826 | 85 | PHE0000148_238 | NAF | 310 | 369 | 116.1 | 9.20E−32 |
| 827 | 86 | PHE0000149_239 | AP2 | 29 | 92 | 128.5 | 1.70E−35 |
| 828 | 87 | PHE0000150_240 | Lectin_legB | 23 | 212 | 8 | 2.60E−11 |
| 828 | 87 | PHE0000150_240 | Lectin_legA | 221 | 265 | 37.6 | 3.80E−08 |
| 828 | 87 | PHE0000150_240 | Pkinase | 344 | 496 | −11.1 | 2.00E−07 |
| 829 | 88 | PHE0000189_282 | HSP20 | 49 | 152 | 169.6 | 7.30E−48 |
| 830 | 89 | PHE0000200_293 | PP2C | 22 | 317 | 296.5 | 4.30E−86 |
| 831 | 90 | PHE0000211_304 | Pkinase | 30 | 288 | 384.1 | 1.90E−112 |
| 832 | 91 | PHE0000213_306 | Pkinase | 64 | 322 | 348.7 | 8.50E−102 |
| 1178 | 437 | PHE0000285_375 | bZIP_1 | 27 | 91 | 50.9 | 3.80E−12 |
| 1178 | 437 | PHE0000285_375 | bZIP_2 | 27 | 81 | 40.8 | 4.00E−09 |
| 833 | 92 | PHE0000368_459 | bZIP_1 | 21 | 85 | 51.2 | 3.10E−12 |
| 833 | 92 | PHE0000368_459 | bZIP_2 | 21 | 75 | 38.5 | 2.00E−08 |
| 834 | 93 | PHE0000369_460 | Pkinase | 17 | 269 | 375.3 | 8.50E−110 |
| 834 | 93 | PHE0000369_460 | Pkinase_Tyr | 17 | 267 | 78.6 | 1.70E−20 |
| 834 | 93 | PHE0000369_460 | UBA | 291 | 330 | 23.4 | 0.0007 |
| 834 | 93 | PHE0000369_460 | KA1 | 459 | 507 | 89.8 | 7.20E−24 |
| 835 | 94 | PHE0000370_461 | Pkinase | 4 | 260 | 298.7 | 9.60E−87 |
| 1340 | 599 | PHE0000667_770 | Acyltransferase | 174 | 337 | 127.2 | 4.00E−35 |
| 1179 | 438 | PHE0000668_771 | Acyltransferase | 207 | 367 | 167.4 | 3.10E−47 |
| 836 | 95 | PHE0000780_853 | SRF-TF | 9 | 59 | 119.7 | 7.20E−33 |
| 836 | 95 | PHE0000780_853 | K-box | 74 | 173 | 147.6 | 3.00E−41 |
| 837 | 96 | PHE0000782_855 | SRF-TF | 9 | 59 | 113.7 | 4.80E−31 |
| 837 | 96 | PHE0000782_855 | K-box | 73 | 174 | 151 | 2.80E−42 |
| 838 | 97 | PHE0000783_856 | SRF-TF | 9 | 59 | 120.2 | 5.30E−33 |
| 838 | 97 | PHE0000783_856 | K-box | 74 | 174 | 145.6 | 1.20E−40 |
| 1341 | 600 | PHE0000784_857 | K-box | 4 | 103 | 147.6 | 3.00E−41 |
| 839 | 98 | PHE0000785_858 | SRF-TF | 9 | 59 | 119.7 | 7.20E−33 |
| 839 | 98 | PHE0000785_858 | K-box | 74 | 173 | 147.6 | 3.00E−41 |
| 1294 | 553 | PHE0000788_861 | SRF-TF | 9 | 59 | 113.7 | 4.80E−31 |
| 1294 | 553 | PHE0000788_861 | K-box | 73 | 174 | 151 | 2.80E−42 |
| 1295 | 554 | PHE0000789_862 | SRF-TF | 9 | 59 | 113.7 | 4.80E−31 |
| 1180 | 439 | PHE0000790_863 | K-box | 4 | 103 | 150.5 | 4.10E−42 |
| 840 | 99 | PHE0000791_864 | SRF-TF | 9 | 59 | 121 | 2.90E−33 |
| 840 | 99 | PHE0000791_864 | K-box | 74 | 173 | 150.5 | 4.10E−42 |
| 841 | 100 | PHE0000792_865 | SRF-TF | 9 | 59 | 121 | 2.90E−33 |
| 842 | 101 | PHE0000794_867 | SRF-TF | 9 | 59 | 120.2 | 5.30E−33 |
| 842 | 101 | PHE0000794_867 | K-box | 74 | 174 | 145.6 | 1.20E−40 |
| 1296 | 555 | PHE0000795_868 | SRF-TF | 9 | 59 | 120.2 | 5.30E−33 |
| 843 | 102 | PHE0000821_896 | DUF6 | 16 | 153 | 32 | 1.90E−06 |
| 843 | 102 | PHE0000821_896 | DUF6 | 190 | 319 | 45.4 | 1.80E−10 |
| 1342 | 601 | PHE0000880_963 | HAMP | 193 | 262 | 64.2 | 3.90E−16 |
| 1342 | 601 | PHE0000880_963 | PAS | 276 | 388 | 36.7 | 7.30E−08 |
| 1342 | 601 | PHE0000880_963 | HisKA | 397 | 465 | 94.4 | 3.00E−25 |
| 1342 | 601 | PHE0000880_963 | HATPase_c | 510 | 629 | 143.2 | 6.10E−40 |
| 1297 | 556 | PHE0000881_964 | HAMP | 200 | 269 | 65.7 | 1.30E−16 |
| 1297 | 556 | PHE0000881_964 | PAS | 283 | 413 | 29.7 | 9.10E−06 |
| 1297 | 556 | PHE0000881_964 | HisKA | 422 | 490 | 95.9 | 1.00E−25 |
| 1297 | 556 | PHE0000881_964 | HATPase_c | 535 | 655 | 138.8 | 1.40E−38 |
| 1343 | 602 | PHE0000883_966 | Response_reg | 276 | 397 | 106.2 | 8.60E−29 |
| 844 | 103 | PHE0001051_1141 | zf-C2H2 | 54 | 76 | 25.1 | 0.00022 |
| 844 | 103 | PHE0001051_1141 | zf-C2H2 | 130 | 152 | 19.2 | 0.014 |
| 845 | 104 | PHE0001053_4292 | NAM | 24 | 155 | 282.4 | 7.60E−82 |
| 846 | 105 | PHE0001054_1144 | KH_1 | 133 | 220 | 13.4 | 0.017 |
| 847 | 106 | PHE0001055_1145 | CBS | 140 | 285 | 37.6 | 3.90E−08 |
| 847 | 106 | PHE0001055_1145 | CBS | 316 | 447 | 48 | 2.80E−11 |
| 849 | 108 | PHE0001057_1147 | Gamma-thionin | 32 | 80 | 77.6 | 3.50E−20 |
| 1344 | 603 | PHE0001063_1153 | CBS | 35 | 175 | 29.2 | 1.30E−05 |
| 1344 | 603 | PHE0001063_1153 | CBS | 193 | 318 | 80.8 | 3.80E−21 |
| 850 | 109 | PHE0001064_1154 | AMPKBI | 304 | 416 | 208.7 | 1.20E−59 |
| 1345 | 604 | PHE0001065_1155 | AMPKBI | 298 | 412 | 210.9 | 2.50E−60 |
| 804 | 63 | PHE0001066_1156 | AMPKBI | 734 | 852 | 209.3 | 7.70E−60 |
| 1346 | 605 | PHE0001069_1159 | bZIP_2 | 78 | 135 | 40.6 | 4.90E−09 |
| 1346 | 605 | PHE0001069_1159 | bZIP_1 | 82 | 142 | 47.6 | 3.70E−11 |
| 851 | 110 | PHE0001071_1161 | AP2 | 68 | 131 | 149.2 | 9.50E−42 |
| 852 | 111 | PHE0001073_1163 | Myb_DNA-binding | 14 | 61 | 44.4 | 3.30E−10 |
| 852 | 111 | PHE0001073_1163 | Myb_DNA-binding | 67 | 112 | 55.5 | 1.60E−13 |
| 1181 | 440 | PHE0001074_1164 | NAM | 19 | 146 | 255.1 | 1.30E−73 |
| 853 | 112 | PHE0001075_1165 | Myb_DNA-binding | 22 | 69 | 47.5 | 4.10E−11 |
| 853 | 112 | PHE0001075_1165 | Myb_DNA-binding | 75 | 120 | 51 | 3.50E−12 |
| 1347 | 606 | PHE0001076_1166 | NAM | 9 | 138 | 293.9 | 2.70E−85 |
| 854 | 113 | PHE0001077_1167 | Myb_DNA-binding | 227 | 278 | 44.3 | 3.60E−10 |
| 1182 | 441 | PHE0001078_1168 | NAM | 8 | 137 | 304.2 | 2.10E−88 |
| 1183 | 442 | PHE0001079_1169 | NAM | 14 | 142 | 309 | 7.60E−90 |
| 1348 | 607 | PHE0001080_1170 | AP2 | 135 | 198 | 137.8 | 2.50E−38 |
| 1184 | 443 | PHE0001082_1172 | NAM | 13 | 143 | 299 | 7.80E−87 |
| 855 | 114 | PHE0001083_1173 | AP2 | 23 | 86 | 147.7 | 2.70E−41 |
| 1185 | 444 | PHE0001085_1175 | HLH | 381 | 430 | 50.8 | 4.10E−12 |
| 1349 | 608 | PHE0001087_1177 | Homeobox | 73 | 127 | 74.6 | 2.90E−19 |
| 1349 | 608 | PHE0001087_1177 | HALZ | 128 | 172 | 81.3 | 2.70E−21 |
| 742 | 1 | PHE0001089_1179 | NAM | 14 | 140 | 289.7 | 4.90E−84 |
| 1350 | 609 | PHE0001094_1184 | zf-C2H2 | 106 | 128 | 19.6 | 0.0097 |
| 856 | 115 | PHE0001095_1185 | bZIP_1 | 23 | 87 | 36.4 | 8.90E−08 |
| 856 | 115 | PHE0001095_1185 | bZIP_2 | 23 | 77 | 28.9 | 1.60E−05 |
| 857 | 116 | PHE0001096_1186 | MFMR | 1 | 201 | 374.1 | 1.90E−109 |
| 857 | 116 | PHE0001096_1186 | bZIP_1 | 303 | 367 | 91.7 | 2.00E−24 |
| 857 | 116 | PHE0001096_1186 | bZIP_2 | 303 | 357 | 31 | 3.80E−06 |
| 858 | 117 | PHE0001097_1187 | bZIP_1 | 42 | 95 | 22.7 | 0.00044 |
| 858 | 117 | PHE0001097_1187 | bZIP_2 | 42 | 92 | 19.3 | 0.0073 |
| 859 | 118 | PHE0001098_1188 | GATA | 160 | 195 | 65.5 | 1.50E−16 |
| 860 | 119 | PHE0001099_1189 | MFMR | 1 | 182 | 333.5 | 3.20E−97 |
| 860 | 119 | PHE0001099_1189 | bZIP_1 | 257 | 321 | 95.2 | 1.80E−25 |
| 860 | 119 | PHE0001099_1189 | bZIP_2 | 257 | 311 | 38.9 | 1.60E−08 |
| 1351 | 610 | PHE0001100_1190 | bZIP_1 | 187 | 245 | 46 | 1.20E−10 |
| 1351 | 610 | PHE0001100_1190 | bZIP_2 | 188 | 241 | 44.6 | 2.90E−10 |
| 861 | 120 | PHE0001101_1191 | GATA | 181 | 216 | 65.5 | 1.50E−16 |
| 862 | 121 | PHE0001107_1197 | Myb_DNA-binding | 24 | 69 | 53.4 | 6.60E−13 |
| 863 | 122 | PHE0001108_1198 | Aminotran_1_2 | 69 | 470 | 61.3 | 2.90E−15 |
| 864 | 123 | PHE0001109_1199 | Aminotran_1_2 | 84 | 469 | 51.8 | 2.00E−12 |
| 865 | 124 | PHE0001110_1200 | Aminotran_1_2 | 61 | 462 | 47.4 | 4.40E−11 |
| 1352 | 611 | PHE0001112_1202 | Aminotran_1_2 | 98 | 491 | 46.2 | 9.80E−11 |
| 1186 | 445 | PHE0001113_1203 | Aminotran_5 | 20 | 363 | 28.9 | 2.40E−09 |
| 866 | 125 | PHE0001115_1205 | Aminotran_1_2 | 92 | 459 | 285.3 | 1.00E−82 |
| 866 | 125 | PHE0001115_1205 | Cys_Met_Meta_PP | 104 | 339 | −275.1 | 0.0034 |
| 867 | 126 | PHE0001120_1210 | Histone | 62 | 137 | 48.9 | 1.50E−11 |
| 867 | 126 | PHE0001120_1210 | CBFD_NFYB_HMF | 68 | 132 | 88.1 | 2.40E−23 |
| 868 | 127 | PHE0001123_1213 | Histone | 92 | 167 | 50.1 | 6.50E−12 |
| 868 | 127 | PHE0001123_1213 | CBFD_NFYB_HMF | 98 | 162 | 89.4 | 1.00E−23 |
| 869 | 128 | PHE0001125_1215 | Histone | 94 | 169 | 51.5 | 2.50E−12 |
| 869 | 128 | PHE0001125_1215 | CBFD_NFYB_HMF | 100 | 164 | 92.7 | 1.00E−24 |
| 870 | 129 | PHE0001126_1216 | Myb_DNA-binding | 123 | 170 | 44.8 | 2.50E−10 |
| 871 | 130 | PHE0001127_1217 | BURP | 299 | 519 | 131.7 | 1.80E−36 |
| 872 | 131 | PHE0001128_1218 | Homeobox | 48 | 103 | 67.3 | 4.40E−17 |
| 872 | 131 | PHE0001128_1218 | HALZ | 104 | 148 | 41.8 | 2.10E−09 |
| 873 | 132 | PHE0001130_1220 | IlvN | 50 | 233 | 112.8 | 9.00E−31 |
| 873 | 132 | PHE0001130_1220 | IlvC | 237 | 522 | 375.3 | 8.50E−110 |
| 1353 | 612 | PHE0001131_1221 | IlvN | 74 | 244 | 189.2 | 8.90E−54 |
| 1353 | 612 | PHE0001131_1221 | IlvC | 248 | 395 | 236.5 | 5.20E−68 |
| 874 | 133 | PHE0001132_1222 | Aminotran_1_2 | 33 | 401 | 489.3 | 4.00E−144 |
| 743 | 2 | PHE0001133_1223 | Aminotran_1_2 | 44 | 419 | 339.8 | 4.20E−99 |
| 744 | 3 | PHE0001134_1224 | Aminotran_1_2 | 44 | 441 | 408.3 | 9.70E−120 |
| 745 | 4 | PHE0001135_1225 | Aminotran_1_2 | 26 | 392 | 492.5 | 4.40E−145 |
| 1187 | 446 | PHE0001136_1226 | Aminotran_1_2 | 31 | 399 | 484.3 | 1.30E−142 |
| 875 | 134 | PHE0001138_1228 | SapB_1 | 59 | 98 | 21.7 | 0.0023 |
| 875 | 134 | PHE0001138_1228 | SapB_2 | 100 | 133 | 23 | 0.00093 |
| 875 | 134 | PHE0001138_1228 | SapB_1 | 145 | 183 | 27.3 | 4.90E−05 |
| 875 | 134 | PHE0001138_1228 | SapB_2 | 186 | 220 | 29.4 | 1.10E−05 |
| 1188 | 447 | PHE0001142_1232 | Methyltransf_3 | 66 | 278 | 415.7 | 5.90E−122 |
| 1354 | 613 | PHE0001143_1233 | HMA | 10 | 70 | 43.3 | 7.20E−10 |
| 876 | 135 | PHE0001145_1235 | F-box | 51 | 104 | 33.3 | 7.30E−07 |
| 877 | 136 | PHE0001146_1236 | Pkinase | 67 | 321 | 269.3 | 6.90E−78 |
| 878 | 137 | PHE0001147_1237 | DUF641 | 22 | 159 | 217.4 | 2.90E−62 |
| 879 | 138 | PHE0001148_1238 | Myb_DNA-binding | 221 | 272 | 48 | 2.80E−11 |
| 1355 | 614 | PHE0001149_1239 | B12D | 2 | 87 | 225.8 | 8.70E−65 |
| 880 | 139 | PHE0001151_1241 | BURP | 355 | 575 | 127.6 | 3.00E−35 |
| 881 | 140 | PHE0001152_1242 | Histone | 58 | 134 | 151.1 | 2.60E−42 |
| 882 | 141 | PHE0001153_1243 | Pkinase | 34 | 292 | 329.8 | 4.30E−96 |
| 883 | 142 | PHE0001154_1244 | Histone | 55 | 127 | 104.9 | 2.10E−28 |
| 885 | 144 | PHE0001157_1247 | DUF760 | 6 | 416 | 592.5 | 3.50E−175 |
| 886 | 145 | PHE0001158_1248 | DUF250 | 243 | 388 | 179.6 | 6.90E−51 |
| 887 | 146 | PHE0001159_1249 | DnaJ | 67 | 134 | 103.9 | 4.20E−28 |
| 888 | 147 | PHE0001167_1257 | YL1 | 11 | 236 | 211.9 | 1.30E−60 |
| 888 | 147 | PHE0001167_1257 | YL1_C | 264 | 293 | 64 | 4.30E−16 |
| 1356 | 615 | PHE0001169_1259 | Chromo | 108 | 158 | 73.2 | 7.20E−19 |
| 889 | 148 | PHE0001171_1261 | Chromo | 62 | 112 | 79.8 | 7.50E−21 |
| 890 | 149 | PHE0001172_1262 | Chromo | 111 | 161 | 73.7 | 5.10E−19 |
| 1357 | 616 | PHE0001176_1266 | Heme_oxygenase | 3 | 205 | 448.6 | 7.40E−132 |
| 1358 | 617 | PHE0001177_1267 | Heme_oxygenase | 3 | 206 | 384.9 | 1.10E−112 |
| 891 | 150 | PHE0001179_1269 | AUX_IAA | 10 | 229 | 299.1 | 7.10E−87 |
| 746 | 5 | PHE0001181_1271 | AUX_IAA | 10 | 228 | 296.6 | 4.10E−86 |
| 1359 | 618 | PHE0001184_1274 | AUX_IAA | 50 | 273 | 324.3 | 1.90E−94 |
| 892 | 151 | PHE0001198_1288 | WRKY | 209 | 267 | 151.2 | 2.40E−42 |
| 892 | 151 | PHE0001198_1288 | WRKY | 381 | 440 | 155.7 | 1.10E−43 |
| 893 | 152 | PHE0001199_1289 | WRKY | 97 | 155 | 144.9 | 1.90E−40 |
| 893 | 152 | PHE0001199_1289 | WRKY | 272 | 331 | 151.5 | 2.00E−42 |
| 894 | 153 | PHE0001208_1298 | AP2 | 119 | 183 | 147.3 | 3.60E−41 |
| 895 | 154 | PHE0001211_1301 | AP2 | 81 | 142 | 117.4 | 3.70E−32 |
| 895 | 154 | PHE0001211_1301 | B3 | 209 | 330 | 110.8 | 3.50E−30 |
| 896 | 155 | PHE0001212_1302 | SAM_2 | 17 | 84 | 65.9 | 1.10E−16 |
| 896 | 155 | PHE0001212_1302 | SAM_1 | 18 | 82 | 36.6 | 7.60E−08 |
| 896 | 155 | PHE0001212_1302 | Pkinase_Tyr | 415 | 710 | 109.7 | 7.70E−30 |
| 896 | 155 | PHE0001212_1302 | Pkinase | 415 | 712 | 292.2 | 8.50E−85 |
| 897 | 156 | PHE0001214_1304 | Pkinase | 400 | 656 | 303.4 | 3.80E−88 |
| 897 | 156 | PHE0001214_1304 | Pkinase_Tyr | 400 | 656 | 147.4 | 3.40E−41 |
| 899 | 158 | PHE0001220_1310 | Pkinase | 40 | 324 | 310.4 | 2.90E−90 |
| 900 | 159 | PHE0001221_1311 | Pkinase | 63 | 347 | 307.5 | 2.10E−89 |
| 901 | 160 | PHE0001225_1315 | Catalase | 18 | 402 | 937.9 | 3.70E−279 |
| 1298 | 557 | PHE0001226_1316 | Catalase | 18 | 401 | 937.7 | 4.30E−279 |
| 747 | 6 | PHE0001227_1317 | Catalase | 27 | 432 | 595.4 | 4.70E−176 |
| 748 | 7 | PHE0001228_1318 | PBD | 336 | 394 | 103.4 | 6.00E−28 |
| 748 | 7 | PHE0001228_1318 | Pkinase | 620 | 871 | 372 | 8.20E−109 |
| 748 | 7 | PHE0001228_1318 | Pkinase_Tyr | 620 | 869 | 109 | 1.30E−29 |
| 902 | 161 | PHE0001238_1328 | Metallothio_2 | 1 | 76 | 138.7 | 1.40E−38 |
| 903 | 162 | PHE0001239_1329 | Pkinase | 67 | 351 | 309.7 | 4.70E−90 |
| 1190 | 449 | PHE0001240_1330 | H_PPase | 9 | 687 | 1072.7 | 0 |
| 904 | 163 | PHE0001241_1331 | YTH | 492 | 582 | 205.8 | 9.10E−59 |
| 905 | 164 | PHE0001242_1332 | Thioredoxin | 31 | 138 | 174.5 | 2.40E−49 |
| 905 | 164 | PHE0001242_1332 | Thioredoxin | 149 | 257 | 187 | 4.20E−53 |
| 905 | 164 | PHE0001242_1332 | ERp29 | 271 | 365 | 172 | 1.30E−48 |
| 1191 | 450 | PHE0001243_1333 | F-box | 53 | 106 | 36.2 | 1.00E−07 |
| 1191 | 450 | PHE0001243_1333 | Tub | 117 | 313 | 182.1 | 1.20E−51 |
| 906 | 165 | PHE0001244_1334 | LIM | 9 | 66 | 54.4 | 3.40E−13 |
| 906 | 165 | PHE0001244_1334 | LIM | 110 | 167 | 66 | 1.10E−16 |
| 907 | 166 | PHE0001245_1335 | W2 | 243 | 319 | 66.1 | 9.70E−17 |
| 908 | 167 | PHE0001246_1337 | zf-C3HC4 | 262 | 302 | 43.2 | 8.10E−10 |
| 749 | 8 | PHE0001247_1338 | Cyclin_N | 67 | 196 | 145.4 | 1.40E−40 |
| 749 | 8 | PHE0001247_1338 | Cyclin_C | 198 | 325 | 52 | 1.80E−12 |
| 1360 | 619 | PHE0001256_1347 | Trypsin | 114 | 309 | 18.9 | 1.80E−05 |
| 1193 | 452 | PHE0001257_1348 | Fer4 | 116 | 139 | 30.6 | 4.90E−06 |
| 1193 | 452 | PHE0001257_1348 | Fer4 | 155 | 178 | 38.5 | 2.10E−08 |
| 910 | 169 | PHE0001258_1349 | Fer4 | 117 | 140 | 28.5 | 1.10E−05 |
| 910 | 169 | PHE0001258_1349 | Fer4 | 156 | 179 | 38.5 | 2.10E−08 |
| 911 | 170 | PHE0001259_1350 | Fer4 | 116 | 139 | 28.6 | 1.00E−05 |
| 911 | 170 | PHE0001259_1350 | Fer4 | 155 | 178 | 38.5 | 2.10E−08 |
| 1362 | 621 | PHE0001265_1356 | efhand | 70 | 98 | 17.9 | 0.034 |
| 1195 | 454 | PHE0001266_1357 | efhand | 77 | 105 | 30.3 | 6.20E−06 |
| 1363 | 622 | PHE0001268_1359 | DUF788 | 1 | 169 | 339.1 | 6.90E−99 |
| 1364 | 623 | PHE0001270_1361 | ERO1 | 64 | 414 | 748.4 | 4.10E−222 |
| 752 | 11 | PHE0001275_1336 | zf-C3HC4 | 257 | 297 | 34.6 | 3.00E−07 |
| 912 | 171 | PHE0001279_1369 | Cystatin | 41 | 129 | 107.9 | 2.60E−29 |
| 913 | 172 | PHE0001280_1370 | Cystatin | 49 | 137 | 80.5 | 4.70E−21 |
| 914 | 173 | PHE0001282_1372 | Chal_sti_synt_C | 342 | 486 | 13.2 | 0.00012 |
| 1196 | 455 | PHE0001283_1373 | ArfGap | 4 | 120 | 173.7 | 4.00E−49 |
| 915 | 174 | PHE0001284_1374 | ArfGap | 4 | 120 | 175.1 | 1.60E−49 |
| 916 | 175 | PHE0001285_1375 | ArfGap | 6 | 122 | 184.5 | 2.30E−52 |
| 1197 | 456 | PHE0001286_1376 | IPK | 17 | 274 | 413.1 | 3.50E−121 |
| 917 | 176 | PHE0001291_1381 | Aa_trans | 27 | 433 | 115.1 | 1.80E−31 |
| 917 | 176 | PHE0001291_1381 | Trp_Tyr_perm | 27 | 368 | −211.7 | 6.50E−05 |
| 918 | 177 | PHE0001292_1382 | Aa_trans | 40 | 446 | 166 | 8.30E−47 |
| 918 | 177 | PHE0001292_1382 | Trp_Tyr_perm | 40 | 443 | −201.1 | 2.20E−05 |
| 919 | 178 | PHE0001297_1387 | DNA_photolyase | 15 | 188 | 237.1 | 3.40E−68 |
| 919 | 178 | PHE0001297_1387 | FAD_binding_7 | 219 | 497 | 502.9 | 3.20E−148 |
| 920 | 179 | PHE0001299_1389 | DNA_photolyase | 5 | 176 | 224 | 2.90E−64 |
| 920 | 179 | PHE0001299_1389 | FAD_binding_7 | 212 | 490 | 464.5 | 1.20E−136 |
| 1198 | 457 | PHE0001301_1391 | Ras | 30 | 192 | 290 | 3.90E−84 |
| 921 | 180 | PHE0001303_1393 | PRA1 | 50 | 203 | 227.8 | 2.20E−65 |
| 1199 | 458 | PHE0001304_1394 | PRA1 | 47 | 197 | 221 | 2.30E−63 |
| 922 | 181 | PHE0001306_1396 | PRA1 | 26 | 177 | 171.5 | 1.90E−48 |
| 1365 | 624 | PHE0001311_1401 | NUDIX | 24 | 160 | 78.9 | 1.50E−20 |
| 923 | 182 | PHE0001312_1402 | NUDIX | 22 | 153 | 62.1 | 1.60E−15 |
| 924 | 183 | PHE0001313_1403 | NUDIX | 19 | 165 | 65 | 2.10E−16 |
| 925 | 184 | PHE0001314_1404 | NUDIX | 19 | 159 | 71.7 | 2.00E−18 |
| 1200 | 459 | PHE0001316_1406 | NUDIX | 20 | 150 | 69.9 | 7.40E−18 |
| 1201 | 460 | PHE0001317_1407 | NUDIX | 19 | 165 | 64.4 | 3.30E−16 |
| 926 | 185 | PHE0001332_1423 | Pkinase | 17 | 272 | 336.5 | 4.00E−98 |
| 926 | 185 | PHE0001332_1423 | NAF | 330 | 389 | 64.3 | 3.50E−16 |
| 927 | 186 | PHE0001333_1424 | Pkinase | 19 | 274 | 341.8 | 1.00E−99 |
| 927 | 186 | PHE0001333_1424 | Pkinase_Tyr | 19 | 274 | 70.1 | 6.30E−18 |
| 927 | 186 | PHE0001333_1424 | NAF | 309 | 369 | 108.9 | 1.30E−29 |
| 928 | 187 | PHE0001334_1425 | Pkinase | 14 | 266 | 382.4 | 6.10E−112 |
| 928 | 187 | PHE0001334_1425 | Pkinase_Tyr | 14 | 264 | 86.3 | 8.10E−23 |
| 928 | 187 | PHE0001334_1425 | UBA | 288 | 327 | 22.3 | 0.0016 |
| 928 | 187 | PHE0001334_1425 | KA1 | 456 | 504 | 101.5 | 2.20E−27 |
| 1300 | 559 | PHE0001335_1426 | Pkinase | 46 | 300 | 359.6 | 4.60E−105 |
| 1300 | 559 | PHE0001335_1426 | NAF | 383 | 440 | 103 | 8.00E−28 |
| 929 | 188 | PHE0001337_1428 | AMPKBI | 190 | 277 | 111.5 | 2.20E−30 |
| 930 | 189 | PHE0001339_1430 | AMPKBI | 194 | 281 | 123.1 | 7.10E−34 |
| 931 | 190 | PHE0001343_1434 | Pkinase | 13 | 268 | 341.6 | 1.20E−99 |
| 931 | 190 | PHE0001343_1434 | NAF | 307 | 367 | 124.7 | 2.30E−34 |
| 932 | 191 | PHE0001346_1437 | Pkinase | 12 | 266 | 351.8 | 1.00E−102 |
| 932 | 191 | PHE0001346_1437 | Pkinase_Tyr | 12 | 264 | 72 | 1.70E−18 |
| 932 | 191 | PHE0001346_1437 | NAF | 313 | 374 | 99.9 | 7.00E−27 |
| 933 | 192 | PHE0001347_1438 | Pkinase | 15 | 269 | 345.1 | 1.00E−100 |
| 933 | 192 | PHE0001347_1438 | Pkinase_Tyr | 15 | 267 | 80.1 | 6.10E−21 |
| 933 | 192 | PHE0001347_1438 | NAF | 319 | 378 | 108.3 | 2.10E−29 |
| 934 | 193 | PHE0001349_1440 | Pkinase | 13 | 268 | 341 | 1.80E−99 |
| 934 | 193 | PHE0001349_1440 | Pkinase_Tyr | 13 | 266 | 74 | 4.30E−19 |
| 934 | 193 | PHE0001349_1440 | NAF | 308 | 369 | 112.8 | 8.60E−31 |
| 935 | 194 | PHE0001350_1441 | Pkinase | 13 | 268 | 334.8 | 1.30E−97 |
| 935 | 194 | PHE0001350_1441 | NAF | 307 | 367 | 132.7 | 8.80E−37 |
| 936 | 195 | PHE0001354_1445 | Pkinase | 21 | 276 | 331.7 | 1.10E−96 |
| 936 | 195 | PHE0001354_1445 | Pkinase_Tyr | 21 | 276 | 65.1 | 8.60E−17 |
| 936 | 195 | PHE0001354_1445 | NAF | 310 | 370 | 94.3 | 3.20E−25 |
| 937 | 196 | PHE0001355_1446 | Pkinase | 17 | 272 | 325.1 | 1.10E−94 |
| 937 | 196 | PHE0001355_1446 | NAF | 300 | 368 | 57.7 | 3.40E−14 |
| 938 | 197 | PHE0001356_1447 | Pkinase | 22 | 283 | 325.8 | 6.90E−95 |
| 938 | 197 | PHE0001356_1447 | NAF | 326 | 385 | 100.6 | 4.10E−27 |
| 939 | 198 | PHE0001357_1448 | Pkinase | 16 | 269 | 302.2 | 8.40E−88 |
| 939 | 198 | PHE0001357_1448 | NAF | 309 | 370 | 98.4 | 1.90E−26 |
| 940 | 199 | PHE0001358_1449 | Pkinase | 14 | 266 | 380.4 | 2.40E−111 |
| 940 | 199 | PHE0001358_1449 | Pkinase_Tyr | 14 | 264 | 84.4 | 3.10E−22 |
| 940 | 199 | PHE0001358_1449 | UBA | 288 | 327 | 22.8 | 0.0011 |
| 940 | 199 | PHE0001358_1449 | KA1 | 454 | 502 | 94.9 | 2.20E−25 |
| 941 | 200 | PHE0001359_1450 | Pkinase_Tyr | 17 | 267 | 72.7 | 1.00E−18 |
| 941 | 200 | PHE0001359_1450 | Pkinase | 17 | 269 | 366.1 | 5.00E−107 |
| 941 | 200 | PHE0001359_1450 | UBA | 291 | 330 | 20.9 | 0.0042 |
| 941 | 200 | PHE0001359_1450 | KA1 | 460 | 508 | 84.8 | 2.40E−22 |
| 942 | 201 | PHE0001360_1451 | CAF1 | 15 | 248 | 399.9 | 3.30E−117 |
| 943 | 202 | PHE0001361_1452 | AP2 | 21 | 84 | 135.2 | 1.60E−37 |
| 944 | 203 | PHE0001362_1453 | GATA | 220 | 255 | 68.2 | 2.30E−17 |
| 945 | 204 | PHE0001363_1455 | NAM | 11 | 135 | 260.6 | 2.80E−75 |
| 946 | 205 | PHE0001364_1456 | CBS | 48 | 262 | 43.1 | 8.40E−10 |
| 946 | 205 | PHE0001364_1456 | CBS | 285 | 422 | 99.1 | 1.20E−26 |
| 947 | 206 | PHE0001375_1467 | WD40 | 159 | 196 | 50.2 | 6.30E−12 |
| 947 | 206 | PHE0001375_1467 | WD40 | 201 | 238 | 34.4 | 3.40E−07 |
| 947 | 206 | PHE0001375_1467 | WD40 | 243 | 280 | 41.8 | 2.10E−09 |
| 947 | 206 | PHE0001375_1467 | WD40 | 285 | 322 | 34.1 | 4.30E−07 |
| 947 | 206 | PHE0001375_1467 | WD40 | 327 | 363 | 15.4 | 0.19 |
| 947 | 206 | PHE0001375_1467 | WD40 | 369 | 405 | 10.4 | 1.4 |
| 947 | 206 | PHE0001375_1467 | WD40 | 419 | 455 | 15.4 | 0.18 |
| 948 | 207 | PHE0001377_1469 | Pkinase | 91 | 407 | 238.5 | 1.30E−68 |
| 949 | 208 | PHE0001380_1472 | HEAT | 82 | 117 | 18.3 | 0.025 |
| 949 | 208 | PHE0001380_1472 | HEAT | 158 | 194 | 23.5 | 0.00069 |
| 949 | 208 | PHE0001380_1472 | HEAT | 197 | 233 | 18.6 | 0.02 |
| 949 | 208 | PHE0001380_1472 | HEAT | 236 | 272 | 32.2 | 1.60E−06 |
| 949 | 208 | PHE0001380_1472 | HEAT | 275 | 311 | 29.8 | 8.50E−06 |
| 949 | 208 | PHE0001380_1472 | HEAT | 314 | 350 | 26.6 | 8.00E−05 |
| 949 | 208 | PHE0001380_1472 | HEAT | 353 | 389 | 28.8 | 1.70E−05 |
| 949 | 208 | PHE0001380_1472 | HEAT | 392 | 428 | 26.4 | 9.20E−05 |
| 949 | 208 | PHE0001380_1472 | HEAT | 431 | 467 | 20 | 0.0074 |
| 949 | 208 | PHE0001380_1472 | HEAT | 470 | 506 | 18 | 0.03 |
| 949 | 208 | PHE0001380_1472 | HEAT | 509 | 545 | 21.7 | 0.0023 |
| 949 | 208 | PHE0001380_1472 | HEAT | 548 | 584 | 22.1 | 0.0018 |
| 1301 | 560 | PHE0001381_1473 | SRF-TF | 9 | 59 | 99.2 | 1.10E−26 |
| 1301 | 560 | PHE0001381_1473 | K-box | 75 | 172 | 79.2 | 1.20E−20 |
| 754 | 13 | PHE0001382_1474 | GRAS | 162 | 502 | 266.8 | 3.80E−77 |
| 950 | 209 | PHE0001385_1477 | Cation_efflux | 1 | 415 | 239.1 | 8.30E−69 |
| 951 | 210 | PHE0001386_1478 | Peptidase_M20 | 107 | 424 | 213.2 | 5.10E−61 |
| 951 | 210 | PHE0001386_1478 | M20_dimer | 214 | 318 | 30.6 | 4.90E−06 |
| 952 | 211 | PHE0001389_1481 | CN_hydrolase | 42 | 219 | 182.1 | 1.20E−51 |
| 953 | 212 | PHE0001392_1484 | Pkinase_Tyr | 89 | 373 | 133.9 | 3.80E−37 |
| 953 | 212 | PHE0001392_1484 | Pkinase | 96 | 373 | 170.8 | 3.10E−48 |
| 954 | 213 | PHE0001394_1486 | SRF-TF | 9 | 59 | 116.9 | 5.10E−32 |
| 954 | 213 | PHE0001394_1486 | K-box | 71 | 174 | 89 | 1.30E−23 |
| 955 | 214 | PHE0001397_1489 | Ran_BP1 | 37 | 158 | 159 | 1.10E−44 |
| 956 | 215 | PHE0001398_1490 | Ran_BP1 | 39 | 160 | 185.6 | 1.10E−52 |
| 957 | 216 | PHE0001405_1497 | NifU_N | 33 | 160 | 273.5 | 3.80E−79 |
| 958 | 217 | PHE0001411_1503 | PSI_PsaF | 49 | 227 | 452.8 | 3.90E−133 |
| 959 | 218 | PHE0001416_1508 | PA | 70 | 171 | 52.7 | 1.10E−12 |
| 959 | 218 | PHE0001416_1508 | Peptidase_A22B | 238 | 524 | 429.6 | 3.80E−126 |
| 960 | 219 | PHE0001417_1509 | NAC | 35 | 92 | 94.3 | 3.20E−25 |
| 962 | 221 | PHE0001421_1513 | ELFV_dehydrog_N | 31 | 161 | 201.2 | 2.10E−57 |
| 962 | 221 | PHE0001421_1513 | ELFV_dehydrog | 176 | 408 | 391.9 | 8.40E−115 |
| 963 | 222 | PHE0001426_1518 | GATA | 91 | 126 | 65.1 | 2.00E−16 |
| 964 | 223 | PHE0001436_1528 | Meth_synt_1 | 2 | 319 | 594.5 | 8.70E−176 |
| 964 | 223 | PHE0001436_1528 | Meth_synt_2 | 433 | 757 | 753.8 | 9.50E−224 |
| 966 | 225 | PHE0001442_1534 | Ubie_methyltran | 40 | 256 | −109 | 0.00017 |
| 966 | 225 | PHE0001442_1534 | CMAS | 42 | 295 | −155.1 | 0.00012 |
| 966 | 225 | PHE0001442_1534 | Methyltransf_11 | 105 | 203 | 105.4 | 1.50E−28 |
| 966 | 225 | PHE0001442_1534 | Methyltransf_12 | 105 | 201 | 49.4 | 1.10E−11 |
| 968 | 227 | PHE0001452_1544 | ADH_N | 30 | 111 | 82.8 | 9.30E−22 |
| 968 | 227 | PHE0001452_1544 | ADH_zinc_N | 142 | 283 | 134 | 3.70E−37 |
| 969 | 228 | PHE0001463_1555 | Homeobox | 18 | 74 | 86.2 | 9.20E−23 |
| 969 | 228 | PHE0001463_1555 | START | 232 | 456 | 280 | 4.20E−81 |
| 805 | 64 | PHE0001471_1563 | Ammonium_transp | 36 | 460 | 578.1 | 7.80E−171 |
| 970 | 229 | PHE0001477_1569 | Myb_DNA-binding | 15 | 62 | 49.1 | 1.30E−11 |
| 970 | 229 | PHE0001477_1569 | Myb_DNA-binding | 68 | 113 | 50.6 | 4.70E−12 |
| 971 | 230 | PHE0001481_1573 | GAF | 195 | 339 | 35.3 | 1.90E−07 |
| 971 | 230 | PHE0001481_1573 | HisKA | 375 | 440 | 36.4 | 8.90E−08 |
| 971 | 230 | PHE0001481_1573 | Response_reg | 641 | 762 | 14.8 | 2.80E−05 |
| 972 | 231 | PHE0001483_1575 | Glyco_hydro_16 | 29 | 210 | 416.9 | 2.60E−122 |
| 972 | 231 | PHE0001483_1575 | XET_C | 234 | 284 | 99.8 | 7.30E−27 |
| 973 | 232 | PHE0001516_1607 | SelR | 87 | 209 | 274.8 | 1.50E−79 |
| 1367 | 626 | PHE0001519_1610 | WD40 | 9 | 47 | 29.3 | 1.20E−05 |
| 1367 | 626 | PHE0001519_1610 | WD40 | 52 | 89 | 34.3 | 3.70E−07 |
| 1367 | 626 | PHE0001519_1610 | WD40 | 94 | 131 | 49.1 | 1.30E−11 |
| 1367 | 626 | PHE0001519_1610 | WD40 | 136 | 173 | 53 | 9.00E−13 |
| 1367 | 626 | PHE0001519_1610 | WD40 | 178 | 215 | 48.8 | 1.60E−11 |
| 1367 | 626 | PHE0001519_1610 | WD40 | 220 | 256 | 8.1 | 2.7 |
| 1302 | 561 | PHE0001521_1612 | Xan_ur_permease | 36 | 443 | 195.6 | 1.00E−55 |
| 974 | 233 | PHE0001522_1613 | Xan_ur_permease | 52 | 463 | 201.3 | 2.10E−57 |
| 1368 | 627 | PHE0001523_1614 | Xan_ur_permease | 31 | 438 | 183.8 | 3.70E−52 |
| 1369 | 628 | PHE0001527_1618 | ELFV_dehydrog_N | 44 | 174 | 245.7 | 8.50E−71 |
| 1369 | 628 | PHE0001527_1618 | ELFV_dehydrog | 190 | 423 | 397.4 | 1.80E−116 |
| 1370 | 629 | PHE0001528_1619 | ELFV_dehydrog_N | 47 | 177 | 253.2 | 4.80E−73 |
| 1370 | 629 | PHE0001528_1619 | ELFV_dehydrog | 193 | 426 | 385.5 | 7.00E−113 |
| 806 | 65 | PHE0001530_1621 | ELFV_dehydrog_N | 43 | 173 | 299.9 | 4.10E−87 |
| 806 | 65 | PHE0001530_1621 | ELFV_dehydrog | 188 | 456 | 446 | 4.50E−131 |
| 755 | 14 | PHE0001531_1622 | ELFV_dehydrog_N | 45 | 178 | 267.3 | 2.70E−77 |
| 755 | 14 | PHE0001531_1622 | ELFV_dehydrog | 194 | 425 | 426.5 | 3.20E−125 |
| 756 | 15 | PHE0001532_1623 | ELFV_dehydrog_N | 39 | 169 | 250.4 | 3.40E−72 |
| 756 | 15 | PHE0001532_1623 | ELFV_dehydrog | 184 | 451 | 456.3 | 3.40E−134 |
| 1371 | 630 | PHE0001533_1624 | ELFV_dehydrog_N | 73 | 203 | 271.9 | 1.20E−78 |
| 1371 | 630 | PHE0001533_1624 | ELFV_dehydrog | 219 | 462 | 480.3 | 2.00E−141 |
| 757 | 16 | PHE0001535_1626 | ELFV_dehydrog_N | 55 | 185 | 277.2 | 3.00E−80 |
| 757 | 16 | PHE0001535_1626 | ELFV_dehydrog | 200 | 443 | 486.1 | 3.80E−143 |
| 758 | 17 | PHE0001536_1627 | ELFV_dehydrog_N | 67 | 197 | 286.3 | 5.10E−83 |
| 758 | 17 | PHE0001536_1627 | ELFV_dehydrog | 213 | 456 | 481.2 | 1.10E−141 |
| 759 | 18 | PHE0001538_1629 | ELFV_dehydrog_N | 33 | 163 | 239.1 | 8.20E−69 |
| 759 | 18 | PHE0001538_1629 | ELFV_dehydrog | 179 | 420 | 400.7 | 1.90E−117 |
| 975 | 234 | PHE0001539_1630 | ELFV_dehydrog_N | 229 | 359 | 216.8 | 4.40E−62 |
| 975 | 234 | PHE0001539_1630 | ELFV_dehydrog | 375 | 622 | 247.2 | 3.10E−71 |
| 1372 | 631 | PHE0001541_1632 | ELFV_dehydrog_N | 245 | 375 | 242.3 | 8.90E−70 |
| 1372 | 631 | PHE0001541_1632 | ELFV_dehydrog | 391 | 638 | 255.7 | 8.70E−74 |
| 1206 | 465 | PHE0001564_1663 | GATase_2 | 2 | 165 | 47 | 1.30E−14 |
| 1206 | 465 | PHE0001564_1663 | Asn_synthase | 214 | 458 | 366.1 | 4.80E−107 |
| 1373 | 632 | PHE0001567_1666 | GATase_2 | 2 | 164 | −6.7 | 1.50E−10 |
| 1373 | 632 | PHE0001567_1666 | Asn_synthase | 241 | 531 | 383.2 | 3.40E−112 |
| 1374 | 633 | PHE0001568_1667 | GATase_2 | 2 | 166 | −20.1 | 1.60E−09 |
| 1374 | 633 | PHE0001568_1667 | Asn_synthase | 249 | 553 | 422.6 | 4.80E−124 |
| 976 | 235 | PHE0001577_1676 | CBS | 48 | 241 | 36.5 | 8.40E−08 |
| 976 | 235 | PHE0001577_1676 | CBS | 264 | 402 | 88.1 | 2.40E−23 |
| 760 | 19 | PHE0001593_1699 | Transketolase_N | 7 | 339 | 846.6 | 1.10E−251 |
| 760 | 19 | PHE0001593_1699 | Transket_pyr | 356 | 533 | 240 | 4.60E−69 |
| 760 | 19 | PHE0001593_1699 | Transketolase_C | 546 | 656 | 73.3 | 6.90E−19 |
| 761 | 20 | PHE0001593_1700 | Transketolase_N | 7 | 339 | 846.6 | 1.10E−251 |
| 761 | 20 | PHE0001593_1700 | Transket_pyr | 356 | 533 | 240 | 4.60E−69 |
| 761 | 20 | PHE0001593_1700 | Transketolase_C | 546 | 656 | 73.3 | 6.90E−19 |
| 977 | 236 | PHE0001602_1713 | Ras | 8 | 179 | 262.7 | 6.60E−76 |
| 978 | 237 | PHE0001604_1715 | Ras | 8 | 179 | 266.7 | 4.10E−77 |
| 979 | 238 | PHE0001605_1716 | Ras | 10 | 181 | 268.9 | 8.80E−78 |
| 980 | 239 | PHE0001606_1717 | Ras | 12 | 183 | 262.9 | 5.60E−76 |
| 981 | 240 | PHE0001610_1721 | WD40 | 7 | 47 | 8.3 | 2.6 |
| 981 | 240 | PHE0001610_1721 | WD40 | 52 | 89 | 30.2 | 6.60E−06 |
| 981 | 240 | PHE0001610_1721 | WD40 | 93 | 130 | 25.8 | 0.00014 |
| 981 | 240 | PHE0001610_1721 | WD40 | 136 | 174 | 10.8 | 1.3 |
| 981 | 240 | PHE0001610_1721 | WD40 | 179 | 214 | 14.7 | 0.3 |
| 981 | 240 | PHE0001610_1721 | WD40 | 219 | 256 | 3.5 | 10 |
| 981 | 240 | PHE0001610_1721 | WD40 | 261 | 298 | 42.6 | 1.20E−09 |
| 1375 | 634 | PHE0001611_1722 | WD40 | 1 | 38 | 12.3 | 0.83 |
| 1375 | 634 | PHE0001611_1722 | WD40 | 43 | 80 | 42.8 | 1.10E−09 |
| 1375 | 634 | PHE0001611_1722 | WD40 | 137 | 174 | 27.1 | 5.60E−05 |
| 1375 | 634 | PHE0001611_1722 | WD40 | 182 | 219 | 27.7 | 3.70E−05 |
| 1375 | 634 | PHE0001611_1722 | WD40 | 279 | 316 | 29.5 | 1.10E−05 |
| 982 | 241 | PHE0001617_1728 | Zip | 60 | 378 | 324.9 | 1.20E−94 |
| 983 | 242 | PHE0001624_1735 | CorA | 16 | 382 | 554.7 | 8.40E−164 |
| 1376 | 635 | PHE0001632_1743 | Na_Ca_ex | 53 | 215 | 62.9 | 9.20E−16 |
| 1376 | 635 | PHE0001632_1743 | Na_Ca_ex | 377 | 531 | 83.8 | 4.60E−22 |
| 985 | 244 | PHE0001642_1753 | zf-C2H2 | 35 | 57 | 17.2 | 0.052 |
| 985 | 244 | PHE0001642_1753 | zf-C2H2 | 84 | 106 | 17.3 | 0.051 |
| 986 | 245 | PHE0001649_1760 | zf-B_box | 2 | 47 | 59.1 | 1.30E−14 |
| 986 | 245 | PHE0001649_1760 | zf-B_box | 54 | 99 | 44.6 | 2.90E−10 |
| 987 | 246 | PHE0002017_2128 | zf-C3HC4 | 332 | 372 | 26.9 | 6.30E−05 |
| 988 | 247 | PHE0002023_2134 | zf-C3HC4 | 336 | 376 | 39.6 | 9.90E−09 |
| 989 | 248 | PHE0002024_2135 | zf-C3HC4 | 338 | 378 | 34.5 | 3.20E−07 |
| 990 | 249 | PHE0002027_2138 | Gln-synt_N | 17 | 98 | 132.2 | 1.20E−36 |
| 990 | 249 | PHE0002027_2138 | Gln-synt_C | 103 | 355 | 330.1 | 3.30E−96 |
| 1377 | 636 | PHE0002029_2140 | DUF246 | 113 | 455 | 712 | 3.70E−211 |
| 991 | 250 | PHE0002041_2151 | ubiquitin | 1 | 74 | 151.2 | 2.50E−42 |
| 991 | 250 | PHE0002041_2151 | Ribosomal_S27 | 101 | 148 | 112.4 | 1.20E−30 |
| 992 | 251 | PHE0002042_2152 | HSP20 | 48 | 152 | 180.1 | 4.80E−51 |
| 993 | 252 | PHE0002049_2159 | RuBisCO_small | 58 | 166 | 249.7 | 5.50E−72 |
| 994 | 253 | PHE0002052_2162 | Lig_chan | 1 | 234 | 179.4 | 7.80E−51 |
| 995 | 254 | PHE0002054_2164 | CN_hydrolase | 31 | 220 | 227.7 | 2.20E−65 |
| 996 | 255 | PHE0002055_2165 | Tic22 | 18 | 280 | 219.3 | 7.70E−63 |
| 997 | 256 | PHE0002067_2177 | Myb_DNA-binding | 14 | 61 | 47.5 | 4.00E−11 |
| 997 | 256 | PHE0002067_2177 | Myb_DNA-binding | 67 | 112 | 55.4 | 1.70E−13 |
| 998 | 257 | PHE0002068_2178 | Myb_DNA-binding | 14 | 61 | 47.5 | 4.00E−11 |
| 998 | 257 | PHE0002068_2178 | Myb_DNA-binding | 67 | 112 | 51.1 | 3.40E−12 |
| 762 | 21 | PHE0002070_2180 | Myb_DNA-binding | 14 | 61 | 47.2 | 5.00E−11 |
| 762 | 21 | PHE0002070_2180 | Myb_DNA-binding | 67 | 112 | 51.9 | 1.90E−12 |
| 999 | 258 | PHE0002071_2181 | Myb_DNA-binding | 14 | 61 | 44.3 | 3.60E−10 |
| 999 | 258 | PHE0002071_2181 | Myb_DNA-binding | 67 | 112 | 54 | 4.30E−13 |
| 1378 | 637 | PHE0002078_2188 | SecY | 108 | 493 | −75.3 | 1.10E−08 |
| 1305 | 564 | PHE0002079_2189 | SecY | 77 | 461 | −18.1 | 2.30E−11 |
| 1000 | 259 | PHE0002080_4290 | SecY | 77 | 461 | −19.2 | 2.60E−11 |
| 1001 | 260 | PHE0002081_2191 | SecY | 77 | 462 | −25.3 | 5.00E−11 |
| 1379 | 638 | PHE0002084_2194 | Ion_trans | 123 | 389 | 69 | 1.30E−17 |
| 1379 | 638 | PHE0002084_2194 | cNMP_binding | 488 | 590 | 40.9 | 4.00E−09 |
| 1002 | 261 | PHE0002088_2198 | UPF0057 | 7 | 58 | 86.8 | 5.70E−23 |
| 1380 | 639 | PHE0002089_2199 | UPF0057 | 29 | 79 | 81.1 | 3.10E−21 |
| 1381 | 640 | PHE0002090_2200 | UPF0057 | 1 | 51 | 104.1 | 3.70E−28 |
| 1207 | 466 | PHE0002094_2204 | Pkinase | 78 | 352 | 113.2 | 6.60E−31 |
| 1207 | 466 | PHE0002094_2204 | Pkinase_Tyr | 78 | 352 | 96.1 | 9.60E−26 |
| 1003 | 262 | PHE0002095_2205 | efhand | 12 | 40 | 41 | 3.60E−09 |
| 1003 | 262 | PHE0002095_2205 | efhand | 48 | 76 | 38.9 | 1.60E−08 |
| 1003 | 262 | PHE0002095_2205 | efhand | 85 | 113 | 42.1 | 1.70E−09 |
| 1003 | 262 | PHE0002095_2205 | efhand | 121 | 149 | 43.2 | 8.10E−10 |
| 1004 | 263 | PHE0002096_2206 | efhand | 12 | 40 | 41 | 3.60E−09 |
| 1004 | 263 | PHE0002096_2206 | efhand | 48 | 76 | 38.9 | 1.60E−08 |
| 1004 | 263 | PHE0002096_2206 | efhand | 85 | 113 | 42.1 | 1.70E−09 |
| 1004 | 263 | PHE0002096_2206 | efhand | 121 | 149 | 43.2 | 8.10E−10 |
| 1005 | 264 | PHE0002099_2209 | efhand | 274 | 302 | 19.3 | 0.013 |
| 1006 | 265 | PHE0002103_2213 | Pkinase | 81 | 358 | 164.6 | 2.20E−46 |
| 1006 | 265 | PHE0002103_2213 | Pkinase_Tyr | 81 | 358 | 129.8 | 6.50E−36 |
| 1208 | 467 | PHE0002121_2229 | Myb_DNA-binding | 76 | 123 | 45.7 | 1.40E−10 |
| 1208 | 467 | PHE0002121_2229 | Myb_DNA-binding | 129 | 174 | 47.2 | 4.90E−11 |
| 1007 | 266 | PHE0002123_2231 | PEPcase | 11 | 966 | 2512.3 | 0 |
| 1008 | 267 | PHE0002125_2233 | PEPcase | 13 | 967 | 2535.1 | 0 |
| 1009 | 268 | PHE0002127_2235 | PEPcase | 11 | 967 | 2556.7 | 0 |
| 763 | 22 | PHE0002128_2236 | PEPcase | 1 | 883 | 767 | 1.00E−227 |
| 1011 | 270 | PHE0002148_2256 | p450 | 38 | 502 | 285.3 | 1.00E−82 |
| 1012 | 271 | PHE0002163_2270 | Yippee | 1 | 104 | 207.1 | 3.60E−59 |
| 1013 | 272 | PHE0002164_2271 | Yippee | 1 | 108 | 207.4 | 2.90E−59 |
| 1014 | 273 | PHE0002183_2290 | HSP20 | 53 | 156 | 186.9 | 4.20E−53 |
| 1306 | 565 | PHE0002185_2292 | HSP20 | 46 | 149 | 183 | 6.50E−52 |
| 1015 | 274 | PHE0002189_2296 | AA_kinase | 15 | 260 | 199.6 | 6.60E−57 |
| 1383 | 642 | PHE0002194_2301 | Aldedh | 52 | 511 | 791.1 | 5.70E−235 |
| 1016 | 275 | PHE0002202_2309 | Annexin | 14 | 80 | 101.3 | 2.60E−27 |
| 1016 | 275 | PHE0002202_2309 | Annexin | 86 | 152 | 89.2 | 1.20E−23 |
| 1016 | 275 | PHE0002202_2309 | Annexin | 169 | 235 | 65.2 | 1.90E−16 |
| 1016 | 275 | PHE0002202_2309 | Annexin | 244 | 310 | 96.1 | 9.50E−26 |
| 1017 | 276 | PHE0002203_2310 | Annexin | 13 | 79 | 99.8 | 7.10E−27 |
| 1017 | 276 | PHE0002203_2310 | Annexin | 85 | 150 | 72.3 | 1.40E−18 |
| 1017 | 276 | PHE0002203_2310 | Annexin | 167 | 233 | 51.8 | 2.00E−12 |
| 1017 | 276 | PHE0002203_2310 | Annexin | 242 | 308 | 85.3 | 1.70E−22 |
| 1018 | 277 | PHE0002204_2311 | Annexin | 14 | 80 | 85.9 | 1.10E−22 |
| 1018 | 277 | PHE0002204_2311 | Annexin | 86 | 153 | 42.1 | 1.70E−09 |
| 1018 | 277 | PHE0002204_2311 | Annexin | 170 | 237 | 101.9 | 1.70E−27 |
| 1018 | 277 | PHE0002204_2311 | Annexin | 246 | 312 | 99.3 | 9.90E−27 |
| 1019 | 278 | PHE0002210_2317 | Kunitz_legume | 33 | 199 | 96.7 | 6.00E−26 |
| 1020 | 279 | PHE0002224_2331 | Di19 | 18 | 236 | 151.1 | 2.60E−42 |
| 1210 | 469 | PHE0002226_2333 | Gamma-thionin | 32 | 80 | 77.6 | 3.50E−20 |
| 1021 | 280 | PHE0002227_2334 | Gamma-thionin | 32 | 78 | 77.9 | 2.90E−20 |
| 1307 | 566 | PHE0002229_2336 | Gamma-thionin | 34 | 80 | 100.7 | 3.90E−27 |
| 1022 | 281 | PHE0002232_2339 | bZIP_2 | 298 | 352 | 48.7 | 1.80E−11 |
| 1022 | 281 | PHE0002232_2339 | bZIP_1 | 298 | 360 | 39.5 | 1.00E−08 |
| 1023 | 282 | PHE0002233_2340 | bZIP_2 | 245 | 299 | 38.7 | 1.80E−08 |
| 1023 | 282 | PHE0002233_2340 | bZIP_1 | 249 | 306 | 39 | 1.40E−08 |
| 1024 | 283 | PHE0002238_2345 | Epimerase | 10 | 253 | 62 | 1.70E−15 |
| 1024 | 283 | PHE0002238_2345 | 3Beta_HSD | 11 | 277 | −51.4 | 2.80E−09 |
| 1024 | 283 | PHE0002238_2345 | NAD_binding_4 | 12 | 237 | 2.9 | 7.20E−09 |
| 1025 | 284 | PHE0002239_2346 | adh_short | 6 | 148 | −26.5 | 0.00023 |
| 1025 | 284 | PHE0002239_2346 | Epimerase | 8 | 253 | 56.9 | 6.00E−14 |
| 1025 | 284 | PHE0002239_2346 | 3Beta_HSD | 9 | 279 | −27.1 | 4.40E−11 |
| 1025 | 284 | PHE0002239_2346 | NAD_binding_4 | 10 | 266 | −24.8 | 4.50E−07 |
| 1026 | 285 | PHE0002240_2347 | adh_short | 3 | 157 | −5.1 | 8.50E−06 |
| 1026 | 285 | PHE0002240_2347 | Epimerase | 5 | 250 | 75.3 | 1.70E−19 |
| 1026 | 285 | PHE0002240_2347 | Polysacc_synt_2 | 5 | 251 | −175.5 | 0.0049 |
| 1026 | 285 | PHE0002240_2347 | 3Beta_HSD | 6 | 276 | −39.5 | 3.70E−10 |
| 1026 | 285 | PHE0002240_2347 | NAD_binding_4 | 7 | 263 | −23.5 | 3.70E−07 |
| 1027 | 286 | PHE0002242_2349 | Epimerase | 7 | 251 | 59 | 1.40E−14 |
| 1027 | 286 | PHE0002242_2349 | NmrA | 7 | 225 | −83.8 | 0.0022 |
| 1027 | 286 | PHE0002242_2349 | 3Beta_HSD | 8 | 273 | −22.7 | 2.10E−11 |
| 1027 | 286 | PHE0002242_2349 | NAD_binding_4 | 9 | 235 | −15.3 | 1.10E−07 |
| 1028 | 287 | PHE0002244_2351 | HSF_DNA-bind | 5 | 178 | 300.9 | 2.10E−87 |
| 1029 | 288 | PHE0002246_2353 | PP2C | 104 | 390 | 288.9 | 8.50E−84 |
| 1030 | 289 | PHE0002254_2361 | adh_short | 22 | 191 | 94.5 | 2.80E−25 |
| 764 | 23 | PHE0002258_69 | PAS | 116 | 230 | 22.8 | 0.0011 |
| 764 | 23 | PHE0002258_69 | PAS | 390 | 504 | 10.5 | 0.036 |
| 764 | 23 | PHE0002258_69 | Pkinase | 582 | 870 | 287.7 | 2.00E−83 |
| 765 | 24 | PHE0002259_68 | PAS | 116 | 230 | 22.8 | 0.0011 |
| 765 | 24 | PHE0002259_68 | PAS | 390 | 504 | 10.5 | 0.036 |
| 765 | 24 | PHE0002259_68 | Pkinase | 582 | 870 | 288.7 | 9.90E−84 |
| 766 | 25 | PHE0002260_70 | PAS | 116 | 230 | 22.8 | 0.0011 |
| 766 | 25 | PHE0002260_70 | PAS | 390 | 504 | 10.5 | 0.036 |
| 766 | 25 | PHE0002260_70 | Pkinase | 582 | 870 | 287.7 | 2.00E−83 |
| 1031 | 290 | PHE0002268_2371 | Pkinase | 30 | 288 | 384.1 | 1.90E−112 |
| 1031 | 290 | PHE0002268_2371 | efhand | 335 | 363 | 39.3 | 1.10E−08 |
| 1031 | 290 | PHE0002268_2371 | efhand | 371 | 399 | 29.9 | 7.90E−06 |
| 1031 | 290 | PHE0002268_2371 | efhand | 407 | 435 | 31.6 | 2.40E−06 |
| 1031 | 290 | PHE0002268_2371 | efhand | 441 | 469 | 33.1 | 8.90E−07 |
| 1032 | 291 | PHE0002269_2372 | Pkinase | 79 | 337 | 343.7 | 2.80E−100 |
| 1032 | 291 | PHE0002269_2372 | efhand | 384 | 412 | 28 | 2.90E−05 |
| 1032 | 291 | PHE0002269_2372 | efhand | 456 | 484 | 23.8 | 0.00056 |
| 1032 | 291 | PHE0002269_2372 | efhand | 490 | 518 | 36.7 | 7.20E−08 |
| 1033 | 292 | PHE0002272_2375 | Pkinase | 73 | 331 | 321.7 | 1.10E−93 |
| 1033 | 292 | PHE0002272_2375 | efhand | 448 | 476 | 25.2 | 0.00021 |
| 1033 | 292 | PHE0002272_2375 | efhand | 482 | 510 | 36 | 1.10E−07 |
| 1211 | 470 | PHE0002293_2395 | Inhibitor_I29 | 36 | 92 | 87.7 | 3.10E−23 |
| 1211 | 470 | PHE0002293_2395 | Peptidase_C1 | 119 | 343 | 408 | 1.20E−119 |
| 1034 | 293 | PHE0002298_2400 | Dehydrin | 21 | 286 | 140 | 5.80E−39 |
| 1035 | 294 | PHE0002299_2401 | Ribonuclease_T2 | 32 | 218 | 96.1 | 9.70E−26 |
| 1036 | 295 | PHE0002321_2422 | Di19 | 9 | 182 | 62.8 | 9.80E−16 |
| 1037 | 296 | PHE0002331_2432 | MIP | 29 | 264 | 432.6 | 4.90E−127 |
| 1212 | 471 | PHE0002338_2439 | MIP | 16 | 237 | 232.5 | 8.10E−67 |
| 1038 | 297 | PHE0002340_2441 | MIP | 31 | 263 | 445.8 | 5.20E−131 |
| 1039 | 298 | PHE0002354_2455 | GST_N | 4 | 77 | 102.5 | 1.10E−27 |
| 1039 | 298 | PHE0002354_2455 | GST_C | 93 | 201 | 107.2 | 4.20E−29 |
| 1041 | 300 | PHE0002361_2462 | PARP | 252 | 445 | −1.5 | 5.00E−07 |
| 1042 | 301 | PHE0002362_2463 | Glycos_transf_2 | 37 | 234 | 90.2 | 5.70E−24 |
| 1043 | 302 | PHE0002364_2465 | Pyr_redox_2 | 23 | 339 | 224.3 | 2.40E−64 |
| 1043 | 302 | PHE0002364_2465 | Pyr_redox | 201 | 293 | 114.9 | 2.10E−31 |
| 1043 | 302 | PHE0002364_2465 | Pyr_redox_dim | 369 | 479 | 170.2 | 4.70E−48 |
| 1044 | 303 | PHE0002365_2466 | HI0933_like | 37 | 358 | −252.7 | 0.0036 |
| 1044 | 303 | PHE0002365_2466 | DAO | 38 | 437 | −32 | 0.0011 |
| 1044 | 303 | PHE0002365_2466 | GIDA | 38 | 362 | −203.5 | 9.40E−05 |
| 1044 | 303 | PHE0002365_2466 | Pyr_redox_2 | 38 | 352 | 244.9 | 1.50E−70 |
| 1044 | 303 | PHE0002365_2466 | Pyr_redox | 209 | 306 | 124.1 | 3.40E−34 |
| 1044 | 303 | PHE0002365_2466 | Pyr_redox_dim | 381 | 490 | 206.4 | 5.80E−59 |
| 1045 | 304 | PHE0002367_2468 | Pyr_redox_2 | 24 | 340 | 235.9 | 8.00E−68 |
| 1045 | 304 | PHE0002367_2468 | Pyr_redox | 202 | 294 | 108.1 | 2.30E−29 |
| 1045 | 304 | PHE0002367_2468 | Pyr_redox_dim | 370 | 480 | 167.2 | 3.60E−47 |
| 1046 | 305 | PHE0002370_2471 | GSHPx | 8 | 117 | 248.2 | 1.50E−71 |
| 1047 | 306 | PHE0002372_2473 | GSHPx | 9 | 118 | 241 | 2.20E−69 |
| 1048 | 307 | PHE0002373_2474 | GSHPx | 11 | 120 | 216.8 | 4.40E−62 |
| 1049 | 308 | PHE0002383_2484 | Cpn60_TCP1 | 40 | 534 | 618.6 | 4.70E−183 |
| 1050 | 309 | PHE0002390_2491 | PMSR | 28 | 181 | 264.3 | 2.30E−76 |
| 1308 | 567 | PHE0002392_2493 | HMA | 6 | 67 | 62.5 | 1.20E−15 |
| 1309 | 568 | PHE0002410_2510 | Pyridoxal_deC | 33 | 383 | 596.4 | 2.30E−176 |
| 1310 | 569 | PHE0002411_2511 | Pyridoxal_deC | 37 | 383 | 354.1 | 2.00E−103 |
| 1051 | 310 | PHE0002414_2514 | Pyridoxal_deC | 33 | 380 | 519.7 | 2.80E−153 |
| 1052 | 311 | PHE0002431_2531 | Rubrerythrin | 133 | 266 | 200.1 | 4.70E−57 |
| 1053 | 312 | PHE0002447_2547 | HSF_DNA-bind | 72 | 234 | 213.5 | 4.20E−61 |
| 1054 | 313 | PHE0002449_2549 | HSF_DNA-bind | 22 | 218 | 162.4 | 1.00E−45 |
| 1055 | 314 | PHE0002459_2559 | F-box | 12 | 57 | 37.3 | 4.60E−08 |
| 1056 | 315 | PHE0002461_2561 | HSF_DNA-bind | 19 | 195 | 351 | 1.70E−102 |
| 1057 | 316 | PHE0002462_2562 | HSF_DNA-bind | 8 | 206 | 179.6 | 6.70E−51 |
| 1058 | 317 | PHE0002464_2564 | HSF_DNA-bind | 9 | 193 | 187.1 | 3.90E−53 |
| 1059 | 318 | PHE0002470_2570 | HSP20 | 50 | 153 | 169.4 | 8.10E−48 |
| 1060 | 319 | PHE0002471_2571 | HSP20 | 49 | 152 | 177.8 | 2.40E−50 |
| 1384 | 643 | PHE0002483_2583 | F-box | 27 | 72 | 22.8 | 0.0011 |
| 1384 | 643 | PHE0002483_2583 | LRR_2 | 182 | 206 | 14.2 | 0.16 |
| 1061 | 320 | PHE0002484_2584 | Sucrose_synth | 6 | 551 | 1368.2 | 0 |
| 1061 | 320 | PHE0002484_2584 | Glycos_transf_1 | 554 | 743 | 100.9 | 3.30E−27 |
| 1311 | 570 | PHE0002485_2585 | Aa_trans | 39 | 424 | 234.8 | 1.70E−67 |
| 1062 | 321 | PHE0002486_2586 | B56 | 64 | 476 | 999.5 | 1.10E−297 |
| 1063 | 322 | PHE0002489_2589 | Cyclin_N | 65 | 197 | 153.6 | 4.80E−43 |
| 1063 | 322 | PHE0002489_2589 | Cyclin_C | 199 | 318 | 33.6 | 6.30E−07 |
| 1064 | 323 | PHE0002490_2590 | Cyclin_N | 61 | 187 | 140.9 | 3.20E−39 |
| 1064 | 323 | PHE0002490_2590 | Cyclin_C | 189 | 312 | 83.5 | 5.70E−22 |
| 1065 | 324 | PHE0002490_3963 | Cyclin_N | 42 | 168 | 140.9 | 3.20E−39 |
| 1065 | 324 | PHE0002490_3963 | Cyclin_C | 170 | 293 | 83.5 | 5.70E−22 |
| 1066 | 325 | PHE0002491_2591 | Cyclin_N | 168 | 293 | 225.4 | 1.10E−64 |
| 1066 | 325 | PHE0002491_2591 | Cyclin_C | 295 | 422 | 168.5 | 1.50E−47 |
| 1385 | 644 | PHE0002494_2594 | Cu-oxidase_3 | 43 | 160 | 228.5 | 1.30E−65 |
| 1385 | 644 | PHE0002494_2594 | Cu-oxidase | 169 | 337 | 251 | 2.10E−72 |
| 1385 | 644 | PHE0002494_2594 | Cu-oxidase_2 | 426 | 564 | 167 | 4.10E−47 |
| 1312 | 571 | PHE0002496_2596 | Aminotran_3 | 83 | 409 | 249.7 | 5.50E−72 |
| 1067 | 326 | PHE0002498_2598 | Pkinase | 139 | 412 | 110.1 | 5.80E−30 |
| 1067 | 326 | PHE0002498_2598 | Pkinase_Tyr | 139 | 412 | 135.1 | 1.70E−37 |
| 1068 | 327 | PHE0002502_2602 | Cpn10 | 54 | 145 | 139.7 | 7.10E−39 |
| 1068 | 327 | PHE0002502_2602 | Cpn10 | 152 | 245 | 150.7 | 3.30E−42 |
| 1069 | 328 | PHE0002506_2606 | Reticulon | 81 | 264 | 295.7 | 7.80E−86 |
| 1070 | 329 | PHE0002507_2607 | Myb_DNA-binding | 49 | 100 | 47.5 | 4.10E−11 |
| 1386 | 645 | PHE0002509_2609 | HSP20 | 46 | 156 | 135.2 | 1.60E−37 |
| 1071 | 330 | PHE0002514_2614 | E1_dh | 150 | 450 | 428.7 | 7.30E−126 |
| 1072 | 331 | PHE0002515_2615 | Hydrolase | 15 | 233 | 45.9 | 1.20E−10 |
| 1387 | 646 | PHE0002523_2623 | Thioredoxin | 104 | 206 | 6.4 | 4.00E−06 |
| 1213 | 472 | PHE0002524_2624 | Thioredoxin | 88 | 195 | 24.7 | 7.30E−08 |
| 1388 | 647 | PHE0002527_2627 | cobW | 11 | 187 | 308.1 | 1.50E−89 |
| 1388 | 647 | PHE0002527_2627 | CobW_C | 228 | 322 | 144.8 | 2.10E−40 |
| 1073 | 332 | PHE0002530_2630 | zf-Dof | 40 | 102 | 137.4 | 3.40E−38 |
| 1074 | 333 | PHE0002532_2632 | Ribosomal_L1 | 9 | 211 | 224.6 | 1.90E−64 |
| 1075 | 334 | PHE0002536_2636 | B3 | 146 | 251 | 105.3 | 1.60E−28 |
| 1075 | 334 | PHE0002536_2636 | Auxin_resp | 273 | 354 | 186.6 | 5.50E−53 |
| 1076 | 335 | PHE0002540_2640 | B3 | 123 | 228 | 108.8 | 1.40E−29 |
| 1076 | 335 | PHE0002540_2640 | Auxin_resp | 250 | 331 | 194 | 3.10E−55 |
| 1076 | 335 | PHE0002540_2640 | AUX_IAA | 593 | 779 | −73.6 | 0.0003 |
| 1389 | 648 | PHE0002546_2646 | DSPc | 36 | 131 | 9.7 | 1.70E−05 |
| 1077 | 336 | PHE0002547_2647 | Abhydrolase_1 | 152 | 377 | 42.4 | 1.40E−09 |
| 1079 | 338 | PHE0002552_2652 | ComA | 20 | 289 | 347 | 2.90E−101 |
| 1390 | 649 | PHE0002557_2657 | AP2 | 15 | 79 | 144.1 | 3.40E−40 |
| 1080 | 339 | PHE0002565_2664 | Metallothio_2 | 2 | 82 | 77.3 | 4.40E−20 |
| 1391 | 650 | PHE0002566_2665 | Ribosomal_L39 | 9 | 51 | 100.6 | 4.20E−27 |
| 1081 | 340 | PHE0002581_2680 | Cpn60_TCP1 | 57 | 561 | 638.5 | 4.90E−189 |
| 1082 | 341 | PHE0002582_2681 | Cpn60_TCP1 | 56 | 559 | 617.9 | 8.00E−183 |
| 1083 | 342 | PHE0002583_2682 | Cpn60_TCP1 | 78 | 582 | 643.9 | 1.20E−190 |
| 1084 | 343 | PHE0002586_2685 | Cpn60_TCP1 | 66 | 570 | 637.4 | 1.10E−188 |
| 1085 | 344 | PHE0002588_2687 | Usp | 6 | 161 | 79 | 1.40E−20 |
| 1086 | 345 | PHE0002591_2690 | DUF393 | 65 | 183 | 109.6 | 8.00E−30 |
| 1087 | 346 | PHE0002596_2695 | Myb_DNA-binding | 59 | 104 | 53.9 | 4.70E−13 |
| 1088 | 347 | PHE0002604_2703 | NPH3 | 213 | 497 | 406 | 4.90E−119 |
| 1215 | 474 | PHE0002607_2706 | Sigma70_r2 | 283 | 356 | 51.5 | 2.50E−12 |
| 1215 | 474 | PHE0002607_2706 | Sigma70_r3 | 359 | 440 | 74.8 | 2.50E−19 |
| 1215 | 474 | PHE0002607_2706 | Sigma70_r4 | 452 | 505 | 75.7 | 1.30E−19 |
| 1089 | 348 | PHE0002608_2707 | Sigma70_r1_2 | 257 | 293 | 34.3 | 3.70E−07 |
| 1089 | 348 | PHE0002608_2707 | Sigma70_r2 | 336 | 406 | 84.7 | 2.60E−22 |
| 1089 | 348 | PHE0002608_2707 | Sigma70_r3 | 410 | 492 | 100.9 | 3.50E−27 |
| 1089 | 348 | PHE0002608_2707 | Sigma70_r4 | 504 | 557 | 90.6 | 4.30E−24 |
| 1216 | 475 | PHE0002609_2708 | Sigma70_r2 | 263 | 333 | 75.4 | 1.50E−19 |
| 1216 | 475 | PHE0002609_2708 | Sigma70_r3 | 337 | 418 | 61.5 | 2.40E−15 |
| 1216 | 475 | PHE0002609_2708 | Sigma70_r4 | 436 | 488 | 41.9 | 2.00E−09 |
| 1313 | 572 | PHE0002611_2710 | Sigma70_r1_2 | 253 | 289 | 29 | 1.50E−05 |
| 1313 | 572 | PHE0002611_2710 | Sigma70_r2 | 332 | 402 | 84.3 | 3.40E−22 |
| 1313 | 572 | PHE0002611_2710 | Sigma70_r3 | 406 | 488 | 96.5 | 7.20E−26 |
| 1313 | 572 | PHE0002611_2710 | Sigma70_r4 | 500 | 553 | 97 | 5.00E−26 |
| 1392 | 651 | PHE0002614_2713 | VQ | 45 | 75 | 43.6 | 5.80E−10 |
| 1090 | 349 | PHE0002615_2714 | VQ | 38 | 68 | 49.3 | 1.10E−11 |
| 1314 | 573 | PHE0002616_2723 | Aa_trans | 26 | 422 | 391.8 | 9.30E−115 |
| 1091 | 350 | PHE0002622_2739 | Myb_DNA-binding | 155 | 205 | 44.2 | 3.90E−10 |
| 1217 | 476 | PHE0002625_2744 | Glycos_transf_1 | 382 | 561 | 50.5 | 5.10E−12 |
| 1092 | 351 | PHE0002629_2748 | Glyco_transf_20 | 77 | 562 | 782.7 | 1.90E−232 |
| 1092 | 351 | PHE0002629_2748 | Trehalose_PPase | 611 | 846 | 311.9 | 1.00E−90 |
| 1093 | 352 | PHE0002634_2753 | Hydrolase | 15 | 204 | 105.6 | 1.30E−28 |
| 1094 | 353 | PHE0002639_2758 | GH3 | 19 | 576 | 880.4 | 7.40E−262 |
| 1095 | 354 | PHE0002640_2759 | GH3 | 8 | 565 | 952 | 2.10E−283 |
| 1096 | 355 | PHE0002643_2762 | GH3 | 12 | 572 | 1048.2 | 0 |
| 1097 | 356 | PHE0002644_2763 | GH3 | 22 | 574 | 1140.9 | 0 |
| 767 | 26 | PHE0002645_2764 | GH3 | 23 | 585 | 1378.6 | 0 |
| 1393 | 652 | PHE0002648_2767 | SRF-TF | 9 | 59 | 109.1 | 1.10E−29 |
| 1393 | 652 | PHE0002648_2767 | K-box | 69 | 169 | 86.4 | 7.90E−23 |
| 1098 | 357 | PHE0002649_2768 | SRF-TF | 9 | 59 | 105.2 | 1.70E−28 |
| 1098 | 357 | PHE0002649_2768 | K-box | 73 | 172 | 120.3 | 5.00E−33 |
| 1394 | 653 | PHE0002652_2771 | NTP_transferase | 9 | 231 | 52.2 | 1.40E−13 |
| 1394 | 653 | PHE0002652_2771 | Hexapep | 294 | 311 | 5.4 | 28 |
| 1394 | 653 | PHE0002652_2771 | Hexapep | 312 | 329 | 15.4 | 0.18 |
| 1394 | 653 | PHE0002652_2771 | Hexapep | 335 | 352 | 13 | 1 |
| 1099 | 358 | PHE0002661_2781 | NTP_transferase | 5 | 275 | 397.9 | 1.40E−116 |
| 1100 | 359 | PHE0002664_2787 | NTP_transferase | 50 | 325 | 377.3 | 2.10E−110 |
| 1315 | 574 | PHE0002670_2793 | NTP_transferase | 3 | 275 | 317 | 3.10E−92 |
| 1101 | 360 | PHE0002688_2821 | Tubulin | 45 | 244 | 380.4 | 2.50E−111 |
| 1101 | 360 | PHE0002688_2821 | Tubulin_C | 246 | 383 | 267.9 | 1.80E−77 |
| 1102 | 361 | PHE0002689_2822 | Tubulin | 49 | 246 | 341.7 | 1.10E−99 |
| 1102 | 361 | PHE0002689_2822 | Tubulin_C | 248 | 393 | 268.1 | 1.60E−77 |
| 1103 | 362 | PHE0002690_2823 | Pkinase | 185 | 469 | 303 | 4.80E−88 |
| 1105 | 364 | PHE0002710_2843 | Ldh_1_N | 6 | 155 | 152.8 | 8.30E−43 |
| 1105 | 364 | PHE0002710_2843 | Ldh_1_C | 157 | 331 | 160.5 | 3.80E−45 |
| 1106 | 365 | PHE0002715_2848 | Thiolase_N | 48 | 306 | 392.7 | 4.90E−115 |
| 1106 | 365 | PHE0002715_2848 | Thiolase_C | 313 | 437 | 235.9 | 7.60E−68 |
| 1107 | 366 | PHE0002717_2850 | EF1_GNE | 140 | 229 | 183.2 | 5.60E−52 |
| 1108 | 367 | PHE0002721_2854 | Glycolytic | 11 | 355 | 936.1 | 1.30E−278 |
| 1109 | 368 | PHE0002724_2859 | Ribosomal_L19e | 2 | 149 | 320.1 | 3.50E−93 |
| 1316 | 575 | PHE0002727_2860 | Sucrose_synth | 11 | 559 | 1379.9 | 0 |
| 1316 | 575 | PHE0002727_2860 | Glycos_transf_1 | 562 | 748 | 111.4 | 2.40E−30 |
| 1110 | 369 | PHE0002728_2861 | MFS_1 | 53 | 486 | 33 | 9.40E−07 |
| 1111 | 370 | PHE0002729_2863 | Ribosomal_L10e | 1 | 176 | 473.5 | 2.30E−139 |
| 1112 | 371 | PHE0002733_2866 | Ribosomal_S17 | 74 | 143 | 147.1 | 4.20E−41 |
| 1113 | 372 | PHE0002734_2867 | Ribosomal_L7Ae | 25 | 120 | 104.7 | 2.50E−28 |
| 1114 | 373 | PHE0002749_2882 | Suc_Fer-like | 55 | 269 | 353.9 | 2.30E−103 |
| 1115 | 374 | PHE0002751_2884 | Ribosomal_S11 | 28 | 146 | 251.9 | 1.20E−72 |
| 1116 | 375 | PHE0002752_2885 | Ribosomal_S2 | 36 | 202 | 314.6 | 1.60E−91 |
| 1117 | 376 | PHE0002771_2904 | Dirigent | 3 | 152 | 37.5 | 3.10E−09 |
| 1117 | 376 | PHE0002771_2904 | Jacalin | 171 | 305 | 116.9 | 5.20E−32 |
| 1118 | 377 | PHE0002790_2925 | p450 | 77 | 520 | 236.7 | 4.40E−68 |
| 1395 | 654 | PHE0002791_2926 | p450 | 73 | 514 | 228.6 | 1.20E−65 |
| 1119 | 378 | PHE0002846_2981 | Trehalose_PPase | 119 | 352 | 316.7 | 3.60E−92 |
| 1396 | 655 | PHE0002849_2984 | UDPGT | 20 | 493 | −63 | 1.70E−07 |
| 1397 | 656 | PHE0002855_2990 | ABC1 | 161 | 279 | 196.7 | 4.80E−56 |
| 1398 | 657 | PHE0002856_2991 | ABC1 | 165 | 283 | 190 | 4.90E−54 |
| 1120 | 379 | PHE0002864_2999 | Pkinase | 4 | 287 | 402 | 7.70E−118 |
| 1121 | 380 | PHE0002869_3004 | Pkinase | 23 | 304 | 338.1 | 1.40E−98 |
| 1122 | 381 | PHE0002875_3010 | DUF581 | 94 | 148 | 82.7 | 1.00E−21 |
| 1399 | 658 | PHE0002886_3021 | DSPc | 30 | 168 | 155.8 | 1.00E−43 |
| 1400 | 659 | PHE0002888_3023 | DSPc | 46 | 179 | 113.5 | 5.40E−31 |
| 1123 | 382 | PHE0002889_3024 | DSPc | 23 | 161 | 152.4 | 1.00E−42 |
| 1401 | 660 | PHE0002893_3028 | DSPc | 159 | 301 | 90.6 | 4.20E−24 |
| 1124 | 383 | PHE0002896_3031 | DSPc | 46 | 184 | 172.7 | 8.00E−49 |
| 1402 | 661 | PHE0002901_3036 | GATA | 472 | 507 | 71.1 | 3.20E−18 |
| 1403 | 662 | PHE0002906_3041 | GATA | 163 | 198 | 62.4 | 1.30E−15 |
| 1404 | 663 | PHE0002910_3045 | Pkinase | 173 | 526 | 105.9 | 1.00E−28 |
| 1125 | 384 | PHE0002918_3053 | Sugar_tr | 65 | 500 | 316.2 | 5.20E−92 |
| 1125 | 384 | PHE0002918_3053 | MFS_1 | 69 | 460 | 62.3 | 1.40E−15 |
| 1405 | 664 | PHE0002923_3058 | VHS | 14 | 161 | 264.6 | 1.70E−76 |
| 1405 | 664 | PHE0002923_3058 | GAT | 231 | 325 | −6 | 0.0035 |
| 1405 | 664 | PHE0002923_3058 | Alpha_adaptinC2 | 437 | 555 | 154.6 | 2.30E−43 |
| 1406 | 665 | PHE0002928_3065 | JmjN | 11 | 63 | 106.6 | 6.40E−29 |
| 1406 | 665 | PHE0002928_3065 | JmjC | 191 | 307 | 184.6 | 2.20E−52 |
| 1406 | 665 | PHE0002928_3065 | zf-C2H2 | 828 | 851 | 24 | 0.00048 |
| 1406 | 665 | PHE0002928_3065 | zf-C2H2 | 857 | 882 | 20.6 | 0.0051 |
| 1218 | 477 | PHE0002939_3089 | Pro_dh | 125 | 474 | 209.4 | 7.10E−60 |
| 807 | 66 | PHE0002940_3090 | Pro_dh | 113 | 464 | 472.4 | 4.80E−139 |
| 1126 | 385 | PHE0002946_3096 | Sugar_tr | 16 | 478 | 52.2 | 1.60E−12 |
| 1126 | 385 | PHE0002946_3096 | MFS_1 | 25 | 444 | 74.4 | 3.20E−19 |
| 1408 | 667 | PHE0002947_3097 | Sugar_tr | 28 | 361 | −47.7 | 1.70E−05 |
| 1408 | 667 | PHE0002947_3097 | MFS_1 | 36 | 382 | 31.8 | 2.20E−06 |
| 1409 | 668 | PHE0002948_3098 | FGGY_N | 22 | 305 | 21.6 | 1.80E−11 |
| 808 | 67 | PHE0002957_3107 | MIP | 339 | 585 | 194.9 | 1.70E−55 |
| 809 | 68 | PHE0002961_3111 | RRM_1 | 197 | 268 | 62.6 | 1.20E−15 |
| 809 | 68 | PHE0002961_3111 | RRM_1 | 542 | 633 | 45.7 | 1.40E−10 |
| 810 | 69 | PHE0002962_3112 | DSPc | 59 | 193 | 198.9 | 1.10E−56 |
| 1127 | 386 | PHE0002963_3113 | DSPc | 1 | 185 | 116 | 9.40E−32 |
| 1128 | 387 | PHE0002966_3116 | DSPc | 696 | 838 | 93.6 | 5.40E−25 |
| 768 | 27 | PHE0002976_3126 | Pkinase | 39 | 351 | 197.5 | 2.90E−56 |
| 1129 | 388 | PHE0002984_3134 | Pkinase | 57 | 323 | 288.3 | 1.30E−83 |
| 1410 | 669 | PHE0002987_3137 | GFO_IDH_MocA | 4 | 127 | 147 | 4.30E−41 |
| 1410 | 669 | PHE0002987_3137 | GFO_IDH_MocA_C | 139 | 251 | 62.5 | 1.20E−15 |
| 1318 | 577 | PHE0002997_3147 | WD40 | 166 | 204 | 6.4 | 4.5 |
| 1318 | 577 | PHE0002997_3147 | WD40 | 235 | 272 | 14.8 | 0.29 |
| 1318 | 577 | PHE0002997_3147 | WD40 | 278 | 316 | 30.9 | 3.90E−06 |
| 1318 | 577 | PHE0002997_3147 | WD40 | 385 | 423 | 38.1 | 2.70E−08 |
| 769 | 28 | PHE0002999_3149 | ADH_N | 26 | 160 | 151.9 | 1.50E−42 |
| 769 | 28 | PHE0002999_3149 | ADH_zinc_N | 189 | 332 | 148 | 2.20E−41 |
| 770 | 29 | PHE0003000_3150 | Aminotran_3 | 29 | 367 | 504.4 | 1.20E−148 |
| 771 | 30 | PHE0003001_3151 | Glyco_hydro_38 | 290 | 560 | 451.7 | 8.40E−133 |
| 771 | 30 | PHE0003001_3151 | Glyco_hydro_38C | 670 | 1080 | 441.3 | 1.20E−129 |
| 772 | 31 | PHE0003002_3152 | TPP_enzyme_N | 25 | 219 | 111.4 | 2.30E−30 |
| 772 | 31 | PHE0003002_3152 | TPP_enzyme_M | 243 | 396 | −7.1 | 0.00019 |
| 772 | 31 | PHE0003002_3152 | TPP_enzyme_C | 443 | 610 | 20.7 | 4.20E−08 |
| 773 | 32 | PHE0003004_3154 | Citrate_synt | 83 | 460 | 749.8 | 1.60E−222 |
| 774 | 33 | PHE0003006_3156 | ALdo_ket_red | 14 | 293 | 471 | 1.30E−138 |
| 775 | 34 | PHE0003007_3157 | PGAM | 11 | 249 | 143.7 | 4.60E−40 |
| 776 | 35 | PHE0003008_3158 | HSP20 | 96 | 207 | 135.9 | 9.80E−38 |
| 1319 | 578 | PHE0003011_3161 | Pro_CA | 45 | 213 | 79.8 | 7.60E−21 |
| 777 | 36 | PHE0003012_3162 | Trehalase_Ca-bi | 106 | 135 | 57.8 | 3.20E−14 |
| 777 | 36 | PHE0003012_3162 | Trehalase | 163 | 721 | 1035.9 | 0 |
| 1411 | 670 | PHE0003017_3167 | Transketolase_N | 7 | 339 | 804.4 | 5.50E−239 |
| 1411 | 670 | PHE0003017_3167 | Transket_pyr | 356 | 533 | 245 | 1.40E−70 |
| 1411 | 670 | PHE0003017_3167 | Transketolase_C | 546 | 657 | 61 | 3.40E−15 |
| 1412 | 671 | PHE0003018_3168 | ubiquitin | 1 | 74 | 149.1 | 1.00E−41 |
| 1412 | 671 | PHE0003018_3168 | ubiquitin | 77 | 150 | 149.1 | 1.00E−41 |
| 1412 | 671 | PHE0003018_3168 | ubiquitin | 153 | 226 | 149.1 | 1.00E−41 |
| 1412 | 671 | PHE0003018_3168 | ubiquitin | 229 | 302 | 149.1 | 1.00E−41 |
| 1412 | 671 | PHE0003018_3168 | ubiquitin | 305 | 378 | 149.1 | 1.00E−41 |
| 779 | 38 | PHE0003022_3172 | Transaldolase | 25 | 329 | 613.5 | 1.60E−181 |
| 1413 | 672 | PHE0003023_3173 | Carb_kinase | 35 | 305 | 496 | 3.80E−146 |
| 1414 | 673 | PHE0003025_3175 | Mpv17_PMP22 | 114 | 184 | 158.9 | 1.20E−44 |
| 1416 | 675 | PHE0003027_3177 | YjeF_N | 24 | 204 | 272.2 | 9.20E−79 |
| 1417 | 676 | PHE0003028_3178 | 2-Hacid_dh | 25 | 113 | 31.6 | 2.50E−06 |
| 1417 | 676 | PHE0003028_3178 | 2-Hacid_dh_C | 124 | 327 | 266.2 | 5.90E−77 |
| 1418 | 677 | PHE0003030_3180 | Asp | 66 | 428 | 180.4 | 4.10E−51 |
| 780 | 39 | PHE0003031_3181 | AMP-binding | 101 | 586 | 262.8 | 6.00E−76 |
| 781 | 40 | PHE0003035_3185 | Hexokinase_1 | 12 | 230 | 456.7 | 2.60E−134 |
| 781 | 40 | PHE0003035_3185 | Hexokinase_2 | 243 | 500 | 501.6 | 8.20E−148 |
| 782 | 41 | PHE0003037_3187 | Aldo_ket_red | 7 | 308 | 501.5 | 8.80E−148 |
| 783 | 42 | PHE0003044_3194 | Pkinase | 47 | 309 | 309.5 | 5.30E−90 |
| 783 | 42 | PHE0003044_3194 | Pkinase_Tyr | 47 | 307 | 91.2 | 2.90E−24 |
| 784 | 43 | PHE0003056_3206 | Pkinase | 352 | 672 | 250.4 | 3.40E−72 |
| 784 | 43 | PHE0003056_3206 | Pkinase_C | 690 | 748 | 33.9 | 4.80E−07 |
| 1219 | 478 | PHE0003057_3207 | Pkinase | 93 | 392 | 231.2 | 1.90E−66 |
| 1219 | 478 | PHE0003057_3207 | Pkinase_C | 410 | 461 | 36.1 | 1.10E−07 |
| 1130 | 389 | PHE0003061_3211 | MatE | 38 | 198 | 128.2 | 2.10E−35 |
| 1130 | 389 | PHE0003061_3211 | MatE | 259 | 422 | 133.6 | 5.00E−37 |
| 1419 | 678 | PHE0003062_3212 | bZIP_1 | 196 | 249 | 28.4 | 2.30E−05 |
| 1419 | 678 | PHE0003062_3212 | bZIP_2 | 196 | 246 | 31.7 | 2.20E−06 |
| 1131 | 390 | PHE0003074_3224 | NTF2 | 10 | 126 | 113.9 | 4.10E−31 |
| 1131 | 390 | PHE0003074_3224 | RRM_1 | 306 | 376 | 39.7 | 8.90E−09 |
| 1420 | 679 | PHE0003088_3240 | Pkinase | 134 | 389 | 335.4 | 8.90E−98 |
| 1420 | 679 | PHE0003088_3240 | Pkinase_C | 409 | 454 | 46.2 | 9.80E−11 |
| 1421 | 680 | PHE0003089_3237 | Pkinase | 140 | 395 | 337.7 | 1.80E−98 |
| 1421 | 680 | PHE0003089_3237 | Pkinase_C | 415 | 460 | 44.2 | 3.90E−10 |
| 1422 | 681 | PHE0003090_3238 | Pkinase | 150 | 406 | 331.2 | 1.60E−96 |
| 1422 | 681 | PHE0003090_3238 | Pkinase_C | 426 | 468 | 33.6 | 5.90E−07 |
| 1423 | 682 | PHE0003091_3239 | Pkinase | 152 | 408 | 324 | 2.40E−94 |
| 1423 | 682 | PHE0003091_3239 | Pkinase_C | 428 | 471 | 48.1 | 2.70E−11 |
| 1133 | 392 | PHE0003101_76 | Globin | 13 | 152 | 113.4 | 5.90E−31 |
| 1132 | 391 | PHE0003101_3969 | Globin | 13 | 152 | 113.4 | 5.90E−31 |
| 1424 | 683 | PHE0003102_3244 | PfkB | 3 | 310 | 292.3 | 8.00E−85 |
| 1425 | 684 | PHE0003102_3579 | PfkB | 3 | 310 | 292.3 | 8.00E−85 |
| 1426 | 685 | PHE0003121_3267 | p450 | 31 | 482 | 286.9 | 3.40E−83 |
| 1134 | 393 | PHE0003124_3270 | APS_kinase | 29 | 184 | 365 | 1.00E−106 |
| 1427 | 686 | PHE0003130_3276 | UDPGT | 9 | 451 | −16.7 | 7.80E−10 |
| 1428 | 687 | PHE0003134_3280 | CH | 15 | 115 | 52.2 | 1.60E−12 |
| 1428 | 687 | PHE0003134_3280 | EB1 | 217 | 264 | 93.1 | 7.50E−25 |
| 1135 | 394 | PHE0003138_3292 | PfkB | 7 | 313 | 371.2 | 1.40E−108 |
| 1136 | 395 | PHE0003139_3295 | PfkB | 18 | 324 | 363.5 | 3.00E−106 |
| 785 | 44 | PHE0003166_3368 | Alpha-amylase | 8 | 429 | −32.5 | 7.70E−08 |
| 1220 | 479 | PHE0003182_3341 | Glycogen_syn | 18 | 661 | 1833.8 | 0 |
| 1137 | 396 | PHE0003190_3389 | HLH | 100 | 151 | 58.5 | 2.00E−14 |
| 786 | 45 | PHE0003191_3390 | HLH | 109 | 160 | 65.5 | 1.50E−16 |
| 1429 | 688 | PHE0003194_3393 | Sina | 130 | 329 | 442 | 6.90E−130 |
| 1430 | 689 | PHE0003195_3394 | Sina | 104 | 303 | 445.2 | 7.60E−131 |
| 1138 | 397 | PHE0003196_3395 | Sina | 105 | 304 | 433.5 | 2.60E−127 |
| 1139 | 398 | PHE0003198_3397 | Sina | 96 | 295 | 414.3 | 1.50E−121 |
| 1431 | 690 | PHE0003199_3398 | Sina | 95 | 294 | 417.1 | 2.10E−122 |
| 1321 | 580 | PHE0003200_3399 | Sina | 102 | 301 | 457.1 | 2.00E−134 |
| 1432 | 691 | PHE0003201_3400 | Sina | 103 | 302 | 434.6 | 1.20E−127 |
| 1433 | 692 | PHE0003208_3410 | DUF250 | 247 | 391 | 172.4 | 1.00E−48 |
| 1140 | 399 | PHE0003211_3417 | H_PPase | 9 | 756 | 1731.2 | 0 |
| 1141 | 400 | PHE0003211_3967 | H_PPase | 9 | 756 | 1731.2 | 0 |
| 1434 | 693 | PHE0003213_3419 | AA_permease | 86 | 546 | 479.9 | 2.80E−141 |
| 1142 | 401 | PHE0003217_3423 | Myb_DNA-binding | 14 | 61 | 49.1 | 1.30E−11 |
| 1142 | 401 | PHE0003217_3423 | Myb_DNA-binding | 67 | 112 | 38.4 | 2.10E−08 |
| 1436 | 695 | PHE0003222_3428 | AA_kinase | 49 | 279 | 234.3 | 2.40E−67 |
| 1436 | 695 | PHE0003222_3428 | PUA | 318 | 392 | 93.1 | 7.60E−25 |
| 1143 | 402 | PHE0003224_3432 | DUF1423 | 102 | 586 | 778.3 | 4.10E−231 |
| 1322 | 581 | PHE0003227_3443 | Histone | 64 | 139 | 44.2 | 4.00E−10 |
| 1322 | 581 | PHE0003227_3443 | CBFD_NFYB_HMF | 70 | 134 | 87.3 | 4.10E−23 |
| 1144 | 403 | PHE0003228_3444 | adh_short | 18 | 186 | 104.4 | 3.00E−28 |
| 1145 | 404 | PHE0003229_3445 | adh_short | 18 | 186 | 103 | 7.70E−28 |
| 1222 | 481 | PHE0003234_3450 | Glycolytic | 11 | 358 | 895.8 | 1.70E−266 |
| 1437 | 696 | PHE0003238_3454 | Glycolytic | 11 | 358 | 927.6 | 4.80E−276 |
| 1223 | 482 | PHE0003239_3455 | FHA | 33 | 108 | 48.2 | 2.40E−11 |
| 1147 | 406 | PHE0003240_3456 | FHA | 32 | 107 | 47.5 | 4.10E−11 |
| 1438 | 697 | PHE0003242_3458 | ABC_tran | 1 | 147 | 111.7 | 2.00E−30 |
| 1148 | 407 | PHE0003243_3459 | ABC_tran | 47 | 233 | 240.5 | 3.20E−69 |
| 1439 | 698 | PHE0003251_3468 | DUF1530 | 110 | 209 | 211.9 | 1.30E−60 |
| 1150 | 409 | PHE0003257_3474 | GRIM-19 | 16 | 141 | 106.8 | 5.50E−29 |
| 1225 | 484 | PHE0003259_3476 | MAP1_LC3 | 14 | 117 | 279.2 | 7.00E−81 |
| 1440 | 699 | PHE0003260_3477 | MAP1_LC3 | 14 | 117 | 274.9 | 1.40E−79 |
| 1323 | 582 | PHE0003261_3478 | MAP1_LC3 | 14 | 117 | 285.4 | 9.50E−83 |
| 787 | 46 | PHE0003266_3485 | ARID | 37 | 146 | 63 | 8.70E−16 |
| 787 | 46 | PHE0003266_3485 | HMG_box | 238 | 305 | 43.6 | 6.20E−10 |
| 1226 | 485 | PHE0003267_3486 | WHEP-TRS | 55 | 107 | 23.7 | 2.60E−05 |
| 1226 | 485 | PHE0003267_3486 | tRNA-synt_2b | 149 | 450 | 262.1 | 9.70E−76 |
| 1226 | 485 | PHE0003267_3486 | HGTP_anticodon | 617 | 705 | 86.2 | 8.80E−23 |
| 1152 | 411 | PHE0003276_3495 | E1_dh | 89 | 393 | 468.9 | 5.60E−138 |
| 1325 | 584 | PHE0003277_3496 | E1_dh | 87 | 392 | 472.3 | 5.20E−139 |
| 815 | 74 | PHE0003280_3499 | DUF6 | 156 | 286 | 34.5 | 3.30E−07 |
| 1153 | 412 | PHE0003282_3501 | DUF6 | 21 | 154 | 47.8 | 3.30E−11 |
| 1153 | 412 | PHE0003282_3501 | DUF6 | 195 | 324 | 31.2 | 3.20E−06 |
| 1229 | 488 | PHE0003285_3504 | PCI | 265 | 364 | 95.1 | 1.90E−25 |
| 1154 | 413 | PHE0003286_3505 | CTP_synth_N | 1 | 287 | 674.4 | 7.50E−200 |
| 1154 | 413 | PHE0003286_3505 | GATase | 311 | 548 | 271.2 | 1.90E−78 |
| 1155 | 414 | PHE0003287_3506 | CTP_synth_N | 1 | 287 | 671.4 | 6.20E−199 |
| 1155 | 414 | PHE0003287_3506 | GATase | 311 | 548 | 264.9 | 1.40E−76 |
| 1156 | 415 | PHE0003304_3524 | BAH | 36 | 156 | 27.2 | 5.00E−05 |
| 1156 | 415 | PHE0003304_3524 | RRM_1 | 366 | 440 | 15.2 | 0.00061 |
| 1444 | 703 | PHE0003308_3527 | Yippee | 1 | 108 | 209.4 | 7.20E−60 |
| 1157 | 416 | PHE0003309_3528 | Yippee | 1 | 108 | 207.1 | 3.70E−59 |
| 1231 | 490 | PHE0003311_3530 | F-box | 25 | 71 | 23.8 | 0.00053 |
| 1231 | 490 | PHE0003311_3530 | FBA_1 | 215 | 418 | 300.3 | 3.10E−87 |
| 1158 | 417 | PHE0003312_3531 | PTR2 | 96 | 499 | 504.3 | 1.20E−148 |
| 1445 | 704 | PHE0003315_3534 | FHA | 32 | 107 | 45.7 | 1.40E−10 |
| 1446 | 705 | PHE0003320_3539 | Pkinase | 45 | 312 | 296.6 | 4.10E−86 |
| 1159 | 418 | PHE0003321_3540 | Pkinase | 44 | 311 | 297.1 | 2.90E−86 |
| 1160 | 419 | PHE0003327_3546 | Pkinase | 4 | 257 | 288.5 | 1.20E−83 |
| 1161 | 420 | PHE0003328_3547 | Pkinase | 4 | 258 | 236.2 | 6.40E−68 |
| 1162 | 421 | PHE0003330_3549 | IF4E | 1 | 198 | 283.9 | 2.70E−82 |
| 1163 | 422 | PHE0003333_3552 | IF4E | 13 | 237 | 411.1 | 1.40E−120 |
| 1164 | 423 | PHE0003333_4250 | IF4E | 13 | 237 | 411.1 | 1.40E−120 |
| 1165 | 424 | PHE0003336_3555 | IF4E | 6 | 235 | 485.9 | 4.10E−143 |
| 1447 | 706 | PHE0003344_3562 | Orn_Arg_deC_N | 73 | 368 | 275.8 | 7.70E−80 |
| 1447 | 706 | PHE0003344_3562 | Orn_DAP_Arg_deC | 371 | 551 | 62.3 | 1.40E−15 |
| 1448 | 707 | PHE0003347_3565 | Acetyltransf_1 | 265 | 343 | 56.1 | 1.10E−13 |
| 1448 | 707 | PHE0003347_3565 | Bromodomain | 460 | 548 | 122.8 | 8.90F-34 |
| 1326 | 585 | PHE0003352_3571 | GAF | 158 | 307 | 90.9 | 3.50E−24 |
| 1326 | 585 | PHE0003352_3571 | HisKA | 343 | 408 | 87.5 | 3.70E−23 |
| 1326 | 585 | PHE0003352_3571 | HATPase_c | 455 | 586 | 123.4 | 5.50E−34 |
| 1166 | 425 | PHE0003353_3572 | Sina | 96 | 295 | 414.3 | 1.50E−121 |
| 1449 | 708 | PHE0003354_3573 | bZIP_1 | 181 | 246 | 21.4 | 0.00062 |
| 789 | 48 | PHE0003358_3581 | p450 | 51 | 492 | 111.4 | 2.30E−30 |
| 1232 | 491 | PHE0003360_3583 | ketoacyl-synt | 126 | 371 | 291.6 | 1.30E−84 |
| 1232 | 491 | PHE0003360_3583 | Ketoacyl-synt_C | 379 | 537 | 218.4 | 1.40E−62 |
| 1167 | 426 | PHE0003361_3584 | malic | 119 | 307 | 392.3 | 6.40E−115 |
| 1167 | 426 | PHE0003361_3584 | Malic_M | 309 | 562 | 459.7 | 3.40E−135 |
| 1168 | 427 | PHE0003362_3585 | Hexokinase_1 | 2 | 203 | 248.6 | 1.20E−71 |
| 1168 | 427 | PHE0003362_3585 | Hexokinase_2 | 210 | 454 | 254.1 | 2.60E−73 |
| 1169 | 428 | PHE0003364_3587 | Aminotran_3 | 1 | 160 | −78.8 | 1.00E−07 |
| 1233 | 492 | PHE0003365_3588 | Rieske | 220 | 317 | 106.9 | 5.30E−29 |
| 1327 | 586 | PHE0003366_3589 | Transaldolase | 78 | 417 | 264.8 | 1.60E−76 |
| 1170 | 429 | PHE0003369_3592 | Gp_dh_N | 4 | 154 | 304.8 | 1.40E−88 |
| 1170 | 429 | PHE0003369_3592 | Gp_dh_C | 159 | 316 | 384.5 | 1.50E−112 |
| 1328 | 587 | PHE0003370_3593 | Cyclotide | 54 | 83 | 48.3 | 2.20E−11 |
| 1171 | 430 | PHE0003373_3596 | Metallophos | 168 | 366 | 87.7 | 3.10E−23 |
| 1234 | 493 | PHE0003374_3597 | Glyco_hydro_16 | 39 | 248 | 321.5 | 1.30E−93 |
| 1234 | 493 | PHE0003374_3597 | XET_C | 280 | 335 | 72.3 | 1.30E−18 |
| 1235 | 494 | PHE0003375_3598 | Trp_syntA | 94 | 347 | 486.6 | 2.60E−143 |
| 1236 | 495 | PHE0003378_3601 | MtN3_slv | 13 | 100 | 138.3 | 1.80E−38 |
| 1236 | 495 | PHE0003378_3601 | MtN3_slv | 134 | 220 | 124 | 3.70E−34 |
| 1450 | 709 | PHE0003380_3603 | Cu-oxidase_3 | 35 | 151 | 248.1 | 1.60E−71 |
| 1450 | 709 | PHE0003380_3603 | Cu-oxidase | 161 | 313 | 207.9 | 2.10E−59 |
| 1450 | 709 | PHE0003380_3603 | Cu-oxidase_2 | 420 | 556 | 190.6 | 3.50E−54 |
| 1172 | 431 | PHE0003381_3604 | F-box | 96 | 141 | 13 | 0.33 |
| 1172 | 431 | PHE0003381_3604 | Kelch_1 | 181 | 223 | 6.9 | 0.59 |
| 1172 | 431 | PHE0003381_3604 | Kelch_1 | 225 | 270 | 44.2 | 3.90E−10 |
| 1172 | 431 | PHE0003381_3604 | Kelch_2 | 225 | 270 | 18.4 | 0.023 |
| 1172 | 431 | PHE0003381_3604 | Kelch_1 | 272 | 319 | 36 | 1.20E−07 |
| 1173 | 432 | PHE0003385_3608 | 3_5_exonuc | 75 | 268 | 75.9 | 1.10E−19 |
| 1173 | 432 | PHE0003385_3608 | KH_1 | 318 | 378 | 41.4 | 2.70E−09 |
| 1239 | 498 | PHE0003387_3610 | DUF125 | 45 | 208 | 196 | 8.10E−56 |
| 1451 | 710 | PHE0003388_3611 | Methyltransf_2 | 1 | 87 | −62.5 | 2.00E−06 |
| 1174 | 433 | PHE0003391_3614 | Pkinase_Tyr | 154 | 428 | 123.4 | 5.60E−34 |
| 1174 | 433 | PHE0003391_3614 | Pkinase | 154 | 428 | 125 | 1.90E−34 |
| 1175 | 434 | PHE0003392_3615 | Lipase_GDSL | 68 | 390 | 191 | 2.50E−54 |
| 1176 | 435 | PHE0003393_3616 | FAD_binding_3 | 68 | 416 | −75.2 | 2.80E−06 |
| 1176 | 435 | PHE0003393_3616 | DAO | 70 | 327 | −18.3 | 0.00014 |
| 1240 | 499 | PHE0003401_3624 | Steroid_dh | 116 | 262 | 187.2 | 3.60E−53 |
| 1456 | 715 | PHE0003414_3654 | Alpha-amylase | 10 | 396 | −67.3 | 8.70E−06 |
| 1457 | 716 | PHE0003415_3655 | Wzy_C | 367 | 433 | 72.1 | 1.50E−18 |
| 790 | 49 | PHE0003416_3656 | UbiA | 106 | 392 | 59.7 | 8.40E−15 |
| 1241 | 500 | PHE0003417_3657 | Myb_DNA-binding | 24 | 73 | 33.8 | 5.40E−07 |
| 1241 | 500 | PHE0003417_3657 | Myb_DNA-binding | 124 | 171 | 46.9 | 6.20E−11 |
| 1242 | 501 | PHE0003418_3658 | ICL | 21 | 551 | 1223.9 | 0 |
| 1458 | 717 | PHE0003431_3639 | Alpha-amylase | 31 | 433 | 275 | 1.30E−79 |
| 1243 | 502 | PHE0003433_3642 | Hydrolase | 7 | 220 | 56.6 | 7.10E−14 |
| 1459 | 718 | PHE0003434_3643 | Hydrolase | 76 | 274 | 119.6 | 8.00E−33 |
| 1460 | 719 | PHE0003436_3645 | Hydrolase | 4 | 214 | 80.7 | 4.20E−21 |
| 1461 | 720 | PHE0003437_3646 | Hydrolase | 21 | 239 | 54.8 | 2.50E−13 |
| 1462 | 721 | PHE0003447_3678 | AT_hook | 41 | 53 | 7.4 | 1.1 |
| 1462 | 721 | PHE0003447_3678 | DUF296 | 68 | 185 | 198.9 | 1.10E−56 |
| 1244 | 503 | PHE0003450_3681 | WRKY | 106 | 165 | 119.4 | 8.80E−33 |
| 791 | 50 | PHE0003457_3688 | Remorin_C | 393 | 504 | 145.6 | 1.20E−40 |
| 1245 | 504 | PHE0003458_3689 | Remorin_C | 402 | 512 | 145 | 1.80E−40 |
| 1246 | 505 | PHE0003459_3690 | zf-C3HC4 | 166 | 207 | 41.3 | 3.00E−09 |
| 1247 | 506 | PHE0003476_3707 | zf-B_box | 3 | 47 | 47.2 | 5.00E−11 |
| 1463 | 722 | PHE0003483_3729 | Ank | 43 | 76 | 17.6 | 0.041 |
| 1463 | 722 | PHE0003483_3729 | Ank | 77 | 107 | 23.3 | 0.00079 |
| 1463 | 722 | PHE0003483_3729 | Ank | 111 | 144 | 26.2 | 0.0001 |
| 1248 | 507 | PHE0003484_3730 | Ank | 28 | 61 | 24.9 | 0.00026 |
| 1249 | 508 | PHE0003491_3737 | SRF-TF | 25 | 75 | 82.8 | 9.50E−22 |
| 1250 | 509 | PHE0003499_3745 | RWP-RK | 526 | 577 | 110.1 | 5.70E−30 |
| 1250 | 509 | PHE0003499_3745 | PB1 | 744 | 827 | 72.3 | 1.30E−18 |
| 792 | 51 | PHE0003502_3748 | zf-C3HC4 | 145 | 186 | 37.6 | 3.70E−08 |
| 793 | 52 | PHE0003506_3752 | Myb_DNA-binding | 65 | 110 | 58.6 | 1.90E−14 |
| 1251 | 510 | PHE0003518_3764 | NAM | 9 | 138 | 304.2 | 2.10E−88 |
| 1464 | 723 | PHE0003519_3765 | NAM | 10 | 139 | 292.4 | 7.70E−85 |
| 794 | 53 | PHE0003522_3768 | NAM | 8 | 143 | 161.4 | 2.10E−45 |
| 1252 | 511 | PHE0003524_3770 | NAM | 18 | 147 | 246.2 | 6.30E−71 |
| 1329 | 588 | PHE0003526_3772 | B3 | 85 | 190 | 110.6 | 4.10E−30 |
| 1329 | 588 | PHE0003526_3772 | Auxin_resp | 255 | 338 | 159.5 | 7.50E−45 |
| 1253 | 512 | PHE0003570_3816 | GRAS | 3 | 352 | 324.6 | 1.50E−94 |
| 1254 | 513 | PHE0003576_3822 | AP2 | 5 | 68 | 129.4 | 9.20E−36 |
| 1330 | 589 | PHE0003581_3827 | F-box | 52 | 105 | 19.8 | 0.0086 |
| 1330 | 589 | PHE0003581_3827 | Tub | 116 | 403 | 582.4 | 3.70E−172 |
| 1465 | 724 | PHE0003582_3828 | ZF-HD_dimer | 179 | 238 | 135.5 | 1.30E−37 |
| 1255 | 514 | PHE0003583_3829 | Myb_DNA-binding | 212 | 263 | 46.2 | 9.90E−11 |
| 1467 | 726 | PHE0003588_3834 | HLH | 146 | 195 | 41.8 | 2.10E−09 |
| 795 | 54 | PHE0003592_3838 | AP2 | 141 | 204 | 126.4 | 7.10E−35 |
| 796 | 55 | PHE0003595_3841 | AP2 | 147 | 211 | 150.9 | 2.90E−42 |
| 1468 | 727 | PHE0003598_3844 | E2F_TDP | 67 | 150 | 145.7 | 1.10E−40 |
| 1257 | 516 | PHE0003599_3845 | E2F_TDP | 5 | 91 | 124.2 | 3.20E−34 |
| 1258 | 517 | PHE0003600_3846 | HLH | 418 | 467 | 51.9 | 1.90E−12 |
| 1469 | 728 | PHE0003606_3852 | HMG_box | 62 | 131 | 73.6 | 5.70E−19 |
| 1470 | 729 | PHE0003613_3860 | Pyridoxal_deC | 33 | 381 | 534.6 | 9.30E−158 |
| 1471 | 730 | PHE0003617_3866 | zf-C2H2 | 80 | 102 | 17.9 | 0.033 |
| 1259 | 518 | PHE0003630_3888 | JmjC | 132 | 240 | 55.4 | 1.70E−13 |
| 1472 | 731 | PHE0003632_3890 | E2F_TDP | 92 | 180 | 149.3 | 9.00E−42 |
| 797 | 56 | PHE0003633_3891 | HLH | 69 | 121 | 58.3 | 2.20E−14 |
| 798 | 57 | PHE0003635_3893 | zf-C2H2 | 209 | 232 | 22 | 0.0019 |
| 1473 | 732 | PHE0003636_3894 | ZF-HD_dimer | 54 | 115 | 134 | 3.60E−37 |
| 1260 | 519 | PHE0003641_3899 | ZF-HD_dimer | 69 | 128 | 136.1 | 8.70E−38 |
| 1261 | 520 | PHE0003644_3902 | HLH | 127 | 178 | 57.7 | 3.40E−14 |
| 1262 | 521 | PHE0003650_3908 | zf-C2H2 | 47 | 69 | 18.7 | 0.018 |
| 1262 | 521 | PHE0003650_3908 | zf-C2H2 | 95 | 117 | 19.7 | 0.0093 |
| 799 | 58 | PHE0003663_3921 | zf-C3HC4 | 329 | 369 | 26 | 0.00012 |
| 800 | 59 | PHE0003670_3928 | AP2 | 20 | 84 | 142.2 | 1.20E−39 |
| 801 | 60 | PHE0003672_3930 | WRKY | 155 | 214 | 135.4 | 1.40E−37 |
| 802 | 61 | PHE0003675_3933 | Response_reg | 16 | 136 | 92.2 | 1.40E−24 |
| 802 | 61 | PHE0003675_3933 | Myb_DNA-binding | 204 | 254 | 48.5 | 2.10E−11 |
| 1474 | 733 | PHE0003676_3934 | bZIP_1 | 192 | 256 | 30.2 | 6.30E−06 |
| 1474 | 733 | PHE0003676_3934 | bZIP_2 | 192 | 246 | 38.6 | 1.90E−08 |
| 1263 | 522 | PHE0003678_3936 | bZIP_1 | 154 | 218 | 31.8 | 2.10E−06 |
| 1263 | 522 | PHE0003678_3936 | bZIP_2 | 154 | 208 | 37.1 | 5.50E−08 |
| 1331 | 590 | PHE0003681_3942 | AP2 | 13 | 77 | 140.4 | 4.20E−39 |
| 1475 | 734 | PHE0003682_3943 | Pkinase | 46 | 300 | 341.2 | 1.60E−99 |
| 1475 | 734 | PHE0003682_3943 | Pkinase_Tyr | 46 | 298 | 66.5 | 7.00E−17 |
| 1475 | 734 | PHE0003682_3943 | NAF | 385 | 442 | 100.7 | 3.70E−27 |
| 1332 | 591 | PHE0003683_3944 | Sugar_tr | 37 | 468 | −65.4 | 6.40E−05 |
| 1332 | 591 | PHE0003683_3944 | MFS_1 | 40 | 463 | 26.4 | 8.80E−05 |
| 1333 | 592 | PHE0003683_3945 | Sugar_tr | 37 | 468 | −65.4 | 6.40E−05 |
| 1333 | 592 | PHE0003683_3945 | MFS_1 | 40 | 463 | 26.4 | 8.80E−05 |
| 1264 | 523 | PHE0003686_3961 | NDK | 80 | 214 | 341.1 | 1.70E−99 |
| 1265 | 524 | PHE0003740_4042 | CBFD_NFYB_HMF | 33 | 98 | 132.1 | 1.40E−36 |
| 1266 | 525 | PHE0003742_4044 | adh_short | 7 | 144 | −11.7 | 2.40E−05 |
| 1266 | 525 | PHE0003742_4044 | RmlD_sub_bind | 8 | 338 | −163.7 | 0.0018 |
| 1266 | 525 | PHE0003742_4044 | Epimerase | 9 | 273 | 266 | 6.90E−77 |
| 1266 | 525 | PHE0003742_4044 | Polysacc_synt_2 | 9 | 330 | −161.6 | 0.0008 |
| 1266 | 525 | PHE0003742_4044 | 3Beta_HSD | 10 | 297 | −69.9 | 6.60E−08 |
| 1266 | 525 | PHE0003742_4044 | NAD_binding_4 | 11 | 251 | −54.1 | 3.60E−05 |
| 1267 | 526 | PHE0003743_4046 | adh_short | 7 | 176 | −21 | 9.70E−05 |
| 1267 | 526 | PHE0003743_4046 | RmlD_sub_bind | 8 | 338 | −131 | 2.10E−05 |
| 1267 | 526 | PHE0003743_4046 | Epimerase | 9 | 273 | 257.7 | 2.10E−74 |
| 1267 | 526 | PHE0003743_4046 | Polysacc_synt_2 | 9 | 323 | −159.7 | 0.00063 |
| 1267 | 526 | PHE0003743_4046 | 3Beta_HSD | 10 | 297 | −57.4 | 7.80E−09 |
| 1267 | 526 | PHE0003743_4046 | NAD_binding_4 | 11 | 248 | −56.6 | 5.20E−05 |
| 1476 | 735 | PHE0003788_4122 | NAM | 2 | 97 | 164.2 | 3.10E−46 |
| 1334 | 593 | PHE0003815_4288 | HAMP | 200 | 269 | 65.7 | 1.30E−16 |
| 1334 | 593 | PHE0003815_4288 | PAS | 283 | 413 | 29.7 | 9.10E−06 |
| 1334 | 593 | PHE0003815_4288 | HisKA | 422 | 490 | 95.9 | 1.00E−25 |
| 1334 | 593 | PHE0003815_4288 | HATPase_c | 535 | 655 | 141.9 | 1.60E−39 |
| 1268 | 527 | PHE0003825_4192 | NAM | 14 | 140 | 297 | 3.10E−86 |
| 1269 | 528 | PHE0003839_4208 | zf-LSD1 | 27 | 51 | 45.6 | 1.50E−10 |
| 1269 | 528 | PHE0003839_4208 | zf-LSD1 | 66 | 90 | 54.8 | 2.50E−13 |
| 1269 | 528 | PHE0003839_4208 | zf-LSD1 | 104 | 128 | 58.4 | 2.10E−14 |
| 1270 | 529 | PHE0003885_4266 | Globin | 14 | 154 | 96.2 | 8.90E−26 |
| 1271 | 530 | PHE0003886_4267 | Globin | 13 | 152 | 113.7 | 4.70E−31 |
| 1477 | 736 | PHE0003899_4284 | PHD | 180 | 227 | 60.2 | 6.20E−15 |
| 1272 | 531 | PHE0003927_4321 | PAD_porph | 14 | 368 | 694.5 | 6.70E−206 |
| 803 | 62 | PHE0003945_4522 | zf-C3HC4 | 146 | 187 | 39.2 | 1.20E−08 |
| 1478 | 737 | PHE0003948_4528 | MFS_1 | 84 | 520 | 28.5 | 2.00E−05 |
| 1273 | 532 | PHE0003959_4544 | Response_reg | 79 | 202 | 93.9 | 4.30E−25 |
| 1273 | 532 | PHE0003959_4544 | CCT | 695 | 736 | 57.3 | 4.60E−14 |
| 1274 | 533 | PHE0003961_4662 | ketoacyl-synt | 47 | 295 | 266.4 | 4.90E−77 |
| 1274 | 533 | PHE0003961_4662 | Ketoacyl-synt_C | 303 | 458 | 209.1 | 9.40E−60 |
| 1275 | 534 | PHE0003965_4553 | bZIP_1 | 277 | 335 | 39.8 | 8.10E−09 |
| 1275 | 534 | PHE0003965_4553 | bZIP_2 | 277 | 334 | 40.9 | 3.80E−09 |
| 1276 | 535 | PHE0003966_4554 | FAE_3-kCoA_syn1 | 20 | 365 | 699 | 3.10E−207 |
| 1277 | 536 | PHE0003977_4565 | HEM4 | 56 | 285 | 85.7 | 1.30E−22 |
| 1479 | 738 | PHE0003981_4568 | PK | 22 | 368 | 809.5 | 1.60E−240 |
| 1479 | 738 | PHE0003981_4568 | PK_C | 382 | 513 | 221.1 | 2.20E−63 |
| 1280 | 539 | PHE0003993_4579 | Na_Ca_ex | 147 | 280 | 119.1 | 1.10E−32 |
| 1338 | 597 | PHE0003995_4581 | Succ_DH_flav_C | 6 | 136 | 265.6 | 8.60E−77 |
| 1283 | 542 | PHE0003997_4583 | Epimerase | 12 | 226 | −40 | 0.001 |
| 1283 | 542 | PHE0003997_4583 | NmrA | 12 | 309 | 475.3 | 6.50E−140 |
| 1481 | 740 | PHE0003998_4584 | Auxin_inducible | 1 | 86 | 77.5 | 3.60E−20 |
| 1287 | 546 | PHE0004003_4589 | Transferase | 22 | 447 | 254.6 | 1.80E−73 |
| 1290 | 549 | PHE0004006_4592 | Abhydrolase_1 | 164 | 425 | 36.8 | 6.60E−08 |
| 1482 | 741 | PHE0004008_4594 | Trypsin | 112 | 272 | 16.7 | 2.50E−05 |
| 1292 | 551 | PHE0004010_4596 | Myb_DNA-binding | 347 | 392 | 43.1 | 8.40E−10 |
| 1339 | 598 | PHE0004022_4657 | PHD | 202 | 252 | 55.9 | 1.20E−13 |

TABLE 12

| | accession | gathering | |
|---|---|---|---|
| Pfam domain name | number | cutoff | domain description |
| 2-Hacid_dh | PF00389.18 | 13.2 | D-isomer specific 2-hydroxyacid |
| | | | dehydrogenase, catalytic domain |
| 2-Hacid_dh_C | PF02826.6 | −75.7 | D-isomer specific 2-hydroxyacid |
| | | | dehydrogenase, NAD binding domain |
| 3Beta_HSD | PF01073.8 | −135.9 | 3-beta hydroxysteroid |
| | | | dehydrogenase/isomerase family |
| 3_5_exonuc | PF01612.10 | −32 | 3′-5′ exonuclease |
| AAA | PF00004.17 | 10 | ATPase family associated with various |
| | | | cellular activities (AAA) |
| AA_kinase | PF00696.16 | −40 | Amino acid kinase family |
| AA_permease | PF00324.10 | −120.8 | Amino acid permease |
| ABC1 | PF03109.6 | −27.6 | ABC1 family |
| ABC_tran | PF00005.14 | 8.6 | ABC transporter |
| ADH_N | PF08240.1 | −14.5 | Alcohol dehydrogenase GroES-like |
| | | | domain |
| ADH_zinc_N | PF00107.15 | 23.8 | Zinc-binding dehydrogenase |
| AMP-binding | PF00501.15 | 0 | AMP-binding enzyme |
| AMPKBI | PF04739.4 | 25 | 5′-AMP-activated protein kinase, beta |
| | | | subunit, complex-interacting region |
| AP2 | PF00847.9 | 0 | AP2 domain |
| APS_kinase | PF01583.9 | 25 | Adenylylsulphate kinase |
| ARID | PF01388.10 | −8 | ARID/BRIGHT DNA binding domain |
| AT_hook | PF02178.7 | 14.2 | AT hook motif |
| AUX_IAA | PF02309.6 | −83 | AUX/IAA family |
| Aa_trans | PF01490.7 | −128.4 | Transmembrane amino acid transporter |
| | | | protein |
| Abhydrolase_1 | PF00561.9 | 5.5 | alpha/beta hydrolase fold |
| Acetyltransf_1 | PF00583.12 | 18.6 | Acetyltransferase (GNAT) family |
| Acyltransferase | PF01553.10 | 6 | Acyltransferase |
| Aldedh | PF00171.11 | −295 | Aldehyde dehydrogenase family |
| Aldo_ket_red | PF00248.10 | −97 | Aldo/keto reductase family |
| Alpha-amylase | PF00128.11 | −93 | Alpha amylase, catalytic domain |
| Alpha_adaptinC2 | PF02883.9 | −12 | Adaptin C-terminal domain |
| Aminotran_1_2 | PF00155.9 | −57.5 | Aminotransferase class I and II |
| Aminotran_3 | PF00202.10 | −207.6 | Aminotransferase class-III |
| Aminotran_5 | PF00266.8 | −92.9 | Aminotransferase class-V |
| Ammonium_transp | PF00909.10 | −144 | Ammonium Transporter Family |
| Ank | PF00023.17 | 21.6 | Ankyrin repeat |
| Annexin | PF00191.8 | 8 | Annexin |
| ArfGap | PF01412.8 | −17 | Putative GTPase activating protein for Arf |
| Asn_synthase | PF00733.10 | −52.8 | Asparagine synthase |
| Asp | PF00026.13 | −186.1 | Eukaryotic aspartyl protease |
| Auxin_inducible | PF02519.4 | −15 | Auxin responsive protein |
| Auxin_resp | PF06507.3 | 25 | Auxin response factor |
| B12D | PF06522.1 | 25 | B12D protein |
| B3 | PF02362.11 | 26.5 | B3 DNA binding domain |
| B56 | PF01603.8 | −210 | Protein phosphatase 2A regulatory B |
| | | | subunit (B56 family) |
| BAH | PF01426.6 | 7 | BAH domain |
| BRO1 | PF03097.6 | 25 | BRO1-like domain |
| BURP | PF03181.5 | −52 | BURP domain |
| Bromodomain | PF00439.13 | 8.9 | Bromodomain |
| CAF1 | PF04857.8 | −100.5 | CAF1 family ribonuclease |
| CBFD_NFYB_HMF | PF00808.12 | 18.4 | Histone-like transcription factor (CBF/NF- |
| | | | Y) and archaeal histone |
| CBS | PF00571.16 | 15.8 | CBS domain pair |
| CCT | PF06203.3 | 25 | CCT motif |
| CH | PF00307.18 | 22.5 | Calponin homology (CH) domain |
| CMAS | PF02353.9 | −177.9 | Cyclopropane-fatty-acyl-phospholipid |
| | | | synthase |
| CN_hydrolase | PF00795.11 | −13.9 | Carbon-nitrogen hydrolase |
| CTP_synth_N | PF06418.2 | 25 | CTP synthase N-terminus |
| CTP_transf_2 | PF01467.15 | −11.8 | Cytidylyltransferase |
| Carb_kinase | PF01256.7 | −66.3 | Carbohydrate kinase |
| Catalase | PF00199.8 | −229 | Catalase |
| Cation_efflux | PF01545.10 | −95.7 | Cation efflux family |
| Chal_sti_synt_C | PF02797.5 | −6.1 | Chalcone and stilbene synthases, C- |
| | | | terminal domain |
| Chromo | PF00385.11 | 27.5 | ‘chromo’ (CHRromatin Organisation |
| | | | MOdifier) domain |
| Citrate_synt | PF00285.10 | −101.5 | Citrate synthase |
| CobW_C | PF07683.3 | 18 | Cobalamin synthesis protein cobW C- |
| | | | terminal domain |
| ComA | PF02679.5 | 25 | (2R)-phospho-3-sulfolactate synthase |
| | | | (ComA) |
| CorA | PF01544.8 | −61.3 | CorA-like Mg2+ transporter protein |
| Cpn10 | PF00166.11 | −7.8 | Chaperonin 10 Kd subunit |
| Cpn60_TCP1 | PF00118.13 | −223.4 | TCP-1/cpn60 chaperonin family |
| Cu-oxidase | PF00394.11 | −18.9 | Multicopper oxidase |
| Cu-oxidase_2 | PF07731.3 | −5.8 | Multicopper oxidase |
| Cu-oxidase_3 | PF07732.4 | 10 | Multicopper oxidase |
| Cyclin_C | PF02984.7 | −13 | Cyclin, C-terminal domain |
| Cyclin_N | PF00134.12 | −14.7 | Cyclin, N-terminal domain |
| Cyclotide | PF03784.3 | 25 | Cyclotide family |
| Cys_Met_Meta_PP | PF01053.9 | −278.4 | Cys/Met metabolism PLP-dependent |
| | | | enzyme |
| Cystatin | PF00031.10 | 17.5 | Cystatin domain |
| DAO | PF01266.11 | −36.5 | FAD dependent oxidoreductase |
| DNA_photolyase | PF00875.7 | −10 | DNA photolyase |
| DSPc | PF00782.9 | −21.8 | Dual specificity phosphatase, catalytic |
| | | | domain |
| DUF125 | PF01988.8 | −10.1 | Integral membrane protein DUF125 |
| DUF1423 | PF07227.1 | 25 | Protein of unknown function (DUF1423) |
| DUF1530 | PF07060.1 | 25 | ProFAR isomerase associated |
| DUF1685 | PF07939.1 | 25 | Protein of unknown function (DUF1685) |
| DUF246 | PF03138.4 | −15 | Plant protein family |
| DUF250 | PF03151.6 | 125 | Domain of unknown function, DUF250 |
| DUF296 | PF03479.4 | −11 | Domain of unknown function (DUF296) |
| DUF393 | PF04134.2 | 25 | Protein of unknown function, DUF393 |
| DUF581 | PF04570.4 | −3.1 | Protein of unknown function (DUF581) |
| DUF6 | PF00892.9 | 30 | Integral membrane protein DUF6 |
| DUF641 | PF04859.2 | 25 | Plant protein of unknown function |
| | | | (DUF641) |
| DUF760 | PF05542.1 | 25 | Protein of unknown function (DUF760) |
| DUF788 | PF05620.1 | 25 | Protein of unknown function (DUF788) |
| Dehydrin | PF00257.8 | −4.4 | Dehydrin |
| Di19 | PF05605.2 | 25 | Drought induced 19 protein (Di19) |
| Dirigent | PF03018.4 | 25 | Dirigent-like protein |
| DnaJ | PF00226.18 | −8 | DnaJ domain |
| E1_dh | PF00676.9 | −90 | Dehydrogenase E1 component |
| E2F_TDP | PF02319.9 | 17 | E2F/DP family winged-helix DNA- |
| | | | binding domain |
| EB1 | PF03271.6 | 25 | EB1-like C-terminal motif |
| EF1_GNE | PF00736.8 | 20 | EF-1 guanine nucleotide exchange domain |
| ELFV_dehydrog | PF00208.10 | −27 | Glutamate/Leucine/Phenylalanine/Valine |
| | | | dehydrogenase |
| ELFV_dehydrog_N | PF02812.7 | 31.8 | Glu/Leu/Phe/Val dehydrogenase, |
| | | | dimerisation domain |
| ERO1 | PF04137.5 | −179.5 | Endoplasmic Reticulum Oxidoreductin 1 |
| | | | (ERO1) |
| ERp29 | PF07749.2 | 10.5 | Endoplasmic reticulum protein ERp29, C- |
| | | | terminal domain |
| Epimerase | PF01370.10 | −46.3 | NAD dependent epimerase/dehydratase |
| | | | family |
| F-box | PF00646.20 | 12.4 | F-box domain |
| FAD_binding_3 | PF01494.8 | −136.6 | FAD binding domain |
| FAD_binding_4 | PF01565.12 | −8.1 | FAD binding domain |
| FAD_binding_7 | PF03441.3 | 25 | FAD binding domain of DNA photolyase |
| FAE_3-kCoA_syn1 | PF07168.1 | 25 | Fatty acid elongase 3-ketoacyl-CoA |
| | | | synthase 1 |
| FA_desaturase | PF00487.13 | −46 | Fatty acid desaturase |
| FBA_1 | PF07734.2 | −39.4 | F-box associated |
| FBPase | PF00316.9 | −170.3 | Fructose-1-6-bisphosphatase |
| FGGY_N | PF00370.10 | −104.7 | FGGY family of carbohydrate kinases, N- |
| | | | terminal domain |
| FHA | PF00498.13 | 25 | FHA domain |
| Fer4 | PF00037.14 | 8 | 4Fe-4S binding domain |
| GAF | PF01590.14 | 23 | GAF domain |
| GAT | PF03127.4 | −7 | GAT domain |
| GATA | PF00320.15 | 28.5 | GATA zinc finger |
| GATase | PF00117.15 | −38.1 | Glutamine amidotransferase class-I |
| GATase_2 | PF00310.10 | −106.2 | Glutamine amidotransferases class-II |
| GFO_IDH_MocA | PF01408.11 | −7.2 | Oxidoreductase family, NAD-binding |
| | | | Rossmann fold |
| GFO_IDH_MocA_C | PF02894.7 | 6 | Oxidoreductase family, C-terminal |
| | | | alpha/beta domain |
| GH3 | PF03321.3 | −336 | GH3 auxin-responsive promoter |
| GIDA | PF01134.11 | −226.7 | Glucose inhibited division protein A |
| GRAS | PF03514.4 | −78 | GRAS family transcription factor |
| GRIM-19 | PF06212.1 | 25 | GRIM-19 protein |
| GSHPx | PF00255.9 | −16 | Glutathione peroxidase |
| GST_C | PF00043.13 | 22.3 | Glutathione S-transferase, C-terminal |
| | | | domain |
| GST_N | PF02798.8 | 14.6 | Glutathione S-transferase, N-terminal |
| | | | domain |
| GTP_EFTU | PF00009.14 | 8 | Elongation factor Tu GTP binding domain |
| GTP_EFTU_D2 | PF03144.13 | 25 | Elongation factor Tu domain 2 |
| GTP_EFTU_D3 | PF03143.6 | 14.3 | Elongation factor Tu C-terminal domain |
| Gamma-thionin | PF00304.10 | 9.6 | Gamma-thionin family |
| Gln-synt_C | PF00120.13 | −124 | Glutamine synthetase, catalytic domain |
| Gln-synt_N | PF03951.8 | 9 | Glutamine synthetase, beta-Grasp domain |
| Globin | PF00042.11 | −8.8 | Globin |
| Glyco_hydro_1 | PF00232.8 | −301.8 | Glycosyl hydrolase family 1 |
| Glyco_hydro_14 | PF01373.7 | −231.4 | Glycosyl hydrolase family 14 |
| Glyco_hydro_16 | PF00722.9 | −65 | Glycosyl hydrolases family 16 |
| Glyco_hydro_38 | PF01074.11 | −125.3 | Glycosyl hydrolases family 38 N-terminal |
| | | | domain |
| Glyco_hydro_38C | PF07748.2 | −93.1 | Glycosyl hydrolases family 38 C-terminal |
| | | | domain |
| Glyco_transf_20 | PF00982.9 | −243.6 | Glycosyltransferase family 20 |
| Glycogen_syn | PF05693.2 | −492.3 | Glycogen synthase |
| Glycolytic | PF00274.8 | −158 | Fructose-bisphosphate aldolase class-I |
| Glycos_transf_1 | PF00534.9 | −7.3 | Glycosyl transferases group 1 |
| Glycos_transf_2 | PF00535.14 | 17.6 | Glycosyl transferase family 2 |
| Glyoxalase | PF00903.14 | 12.1 | Glyoxalase/Bleomycin resistance |
| | | | protein/Dioxygenase superfamily |
| Got1 | PF04178.2 | 25 | Got1-like family |
| Gp_dh_C | PF02800.8 | −64.1 | Glyceraldehyde 3-phosphate |
| | | | dehydrogenase, C-terminal domain |
| Gp_dh_N | PF00044.11 | −74.2 | Glyceraldehyde 3-phosphate |
| | | | dehydrogenase, NAD binding domain |
| HALZ | PF02183.7 | 17 | Homeobox associated leucine zipper |
| HAMP | PF00672.13 | 17 | HAMP domain |
| HATPase_c | PF02518.13 | 22.4 | Histidine kinase-, DNA gyrase B-, and |
| | | | HSP90-like ATPase |
| HEAT | PF02985.9 | 17.6 | HEAT repeat |
| HEM4 | PF02602.5 | −12 | Uroporphyrinogen-III synthase HemD |
| HGTP_anticodon | PF03129.9 | −2 | Anticodon binding domain |
| HI0933_like | PF03486.4 | −255.8 | HI0933-like protein |
| HLH | PF00010.15 | 8.2 | Helix-loop-helix DNA-binding domain |
| HMA | PF00403.14 | 17.4 | Heavy-metal-associated domain |
| HMG_box | PF00505.8 | 4.1 | HMG (high mobility group) box |
| HSF_DNA-bind | PF00447.7 | −70 | HSF-type DNA-binding |
| HSP20 | PF00011.9 | 13 | Hsp20/alpha crystallin family |
| H_PPase | PF03030.5 | −377 | Inorganic H+ pyrophosphatase |
| Heme_oxygenase | PF01126.10 | −58 | Heme oxygenase |
| Hexapep | PF00132.11 | 20 | Bacterial transferase hexapeptide (three |
| | | | repeats) |
| Hexokinase_1 | PF00349.10 | −110.3 | Hexokinase |
| Hexokinase_2 | PF03727.5 | −131.3 | Hexokinase |
| HisKA | PF00512.13 | 10.2 | His Kinase A (phosphoacceptor) domain |
| Hist_deacetyl | PF00850.9 | −71 | Histone deacetylase domain |
| Histone | PF00125.12 | 17.4 | Core histone H2A/H2B/H3/H4 |
| Homeobox | PF00046.17 | −4.1 | Homeobox domain |
| Hpt | PF01627.11 | 25 | Hpt domain |
| Hydrolase | PF00702.13 | 13.6 | haloacid dehalogenase-like hydrolase |
| ICL | PF00463.9 | −234 | Isocitrate lyase family |
| IF4E | PF01652.8 | −35 | Eukaryotic initiation factor 4E |
| IPK | PF03770.6 | 25 | Inositol polyphosphate kinase |
| I1vC | PF01450.8 | −33.8 | Acetohydroxy acid isomeroreductase, |
| | | | catalytic domain |
| I1vN | PF07991.1 | −75.8 | Acetohydroxy acid isomeroreductase, |
| | | | catalytic domain |
| Inhibitor_I29 | PF08246.1 | 4.9 | Cathepsin propeptide inhibitor domain |
| | | | (I29) |
| Ion_trans | PF00520.18 | −4.5 | Ion transport protein |
| Isoamylase_N | PF02922.7 | −6.5 | Isoamylase N-terminal domain |
| Jacalin | PF01419.6 | 20 | Jacalin-like lectin domain |
| JmjC | PF02373.11 | −8 | JmjC domain |
| JmjN | PF02375.6 | 25 | jmjN domain |
| K-box | PF01486.7 | 0 | K-box region |
| KA1 | PF02149.9 | 25 | Kinase associated domain 1 |
| KH_1 | PF00013.17 | 8.1 | KH domain |
| Kelch_1 | PF01344.13 | 20 | Kelch motif |
| Kelch_2 | PF07646.4 | 20 | Kelch motif |
| Ketoacyl-synt_C | PF02801.10 | −54.9 | Beta-ketoacyl synthase, C-terminal |
| | | | domain |
| Kunitz_legume | PF00197.8 | −32 | Trypsin and protease inhibitor |
| LEA_5 | PF00477.7 | 25 | Small hydrophilic plant seed protein |
| LIM | PF00412.10 | 0 | LIM domain |
| LRR_2 | PF07723.2 | 8.7 | Leucine Rich Repeat |
| Lactamase_B | PF00753.15 | 22.3 | Metallo-beta-lactamase superfamily |
| Ldh_1_C | PF02866.6 | −13 | lactate/malate dehydrogenase, alpha/beta |
| | | | C-terminal domain |
| Ldh_1_N | PF00056.11 | −31.3 | lactate/malate dehydrogenase, NAD |
| | | | binding domain |
| Lectin_legA | PF00138.7 | 19 | Legume lectins alpha domain |
| Lectin_legB | PF00139.9 | −77 | Legume lectins beta domain |
| Lig_chan | PF00060.16 | 8.2 | Ligand-gated ion channel |
| Lipase_GDSL | PF00657.11 | 10.9 | GDSL-like Lipase/Acylhydrolase |
| M20_dimer | PF07687.3 | 12 | Peptidase dimerisation domain |
| MAP1_LC3 | PF02991.5 | −18.8 | Microtubule associated protein 1A/1B, |
| | | | light chain 3 |
| MFMR | PF07777.1 | −46.7 | G-box binding protein MFMR |
| MFS_1 | PF07690.4 | 23.5 | Major Facilitator Superfamily |
| MIP | PF00230.8 | −62 | Major intrinsic protein |
| Malic_M | PF03949.4 | −143.9 | Malic enzyme, NAD binding domain |
| MatE | PF01554.8 | 59.6 | MatE |
| Metallophos | PF00149.16 | 22 | Calcineurin-like phosphoesterase |
| Metallothio_2 | PF01439.7 | −3 | Metallothionein |
| Meth_synt_1 | PF08267.1 | −167.8 | Cobalamin-independent synthase, N- |
| | | | terminal domain |
| Meth_synt_2 | PF01717.7 | −155 | Cobalamin-independent synthase, |
| | | | Catalytic domain |
| Methyltransf_11 | PF08241.1 | 17.1 | Methyltransferase domain |
| Methyltransf_12 | PF08242.1 | 21.4 | Methyltransferase domain |
| Methyltransf_2 | PF00891.7 | −103.8 | O-methyltransferase |
| Methyltransf_3 | PF01596.7 | −120.6 | O-methyltransferase |
| Mpv17_PMP22 | PF04117.2 | −5.4 | Mpv17/PMP22 family |
| MtN3_slv | PF03083.5 | −0.8 | MtN3/saliva family |
| Myb_DNA-binding | PF00249.18 | 19.1 | Myb-like DNA-binding domain |
| NAC | PF01849.6 | 0 | NAC domain |
| NAD_binding_4 | PF07993.1 | −87.7 | Male sterility protein |
| NAF | PF03822.4 | 25 | NAF domain |
| NAM | PF02365.5 | −19 | No apical meristem (NAM) protein |
| NDK | PF00334.8 | −59.9 | Nucleoside diphosphate kinase |
| NIF | PF03031.7 | −81 | NL1 interacting factor-like phosphatase |
| NPH3 | PF03000.4 | 25 | NPH3 family |
| NTF2 | PF02136.10 | 6 | Nuclear transport factor 2 (NTF2) domain |
| NTP_transferase | PF00483.12 | −90.5 | Nucleotidyl transferase |
| NUDIX | PF00293.16 | 0 | NUDIX domain |
| Na_Ca_ex | PF01699.12 | 25 | Sodium/calcium exchanger protein |
| NifU_N | PF01592.6 | −13 | NifU-like N terminal domain |
| NmrA | PF05368.2 | −90.6 | NmrA-like family |
| Orn_Arg_deC_N | PF02784.6 | −76 | Pyridoxal-dependent decarboxylase, |
| | | | pyridoxal binding domain |
| Orn_DAP_Arg_deC | PF00278.11 | −34.9 | Pyridoxal-dependent decarboxylase, C- |
| | | | terminal sheet domain |
| Oxidored_FMN | PF00724.8 | −147.7 | NADH:flavin oxidoreductase/NADH |
| | | | oxidase family |
| PA | PF02225.10 | 13 | PA domain |
| PAD_porph | PF04371.4 | −180.8 | Porphyromonas-type peptidyl-arginine |
| | | | deiminase |
| PARP | PF00644.9 | −55.5 | Poly(ADP-ribose) polymerase catalytic |
| | | | domain |
| PAS | PF00989.12 | 20 | PAS fold |
| PB1 | PF00564.12 | 12.1 | PB1 domain |
| PBD | PF00786.16 | 12.1 | P21-Rho-binding domain |
| PCI | PF01399.14 | 25 | PCI domain |
| PDZ | PF00595.11 | 12.1 | PDZ domain (Also known as DHR or |
| | | | GLGF) |
| PEP-utilizers | PF00391.12 | 10 | PEP-utilising enzyme, mobile domain |
| PEP-utilizers_C | PF02896.7 | −173 | PEP-utilising enzyme, TIM barrel domain |
| PEPcase | PF00311.7 | 25 | Phosphoenolpyruvate carboxylase |
| PGAM | PF00300.11 | −3 | Phosphoglycerate mutase family |
| PHD | PF00628.16 | 25.9 | PHD-finger |
| PK | PF00224.10 | −244 | Pyruvate kinase, barrel domain |
| PK_C | PF02887.5 | −44 | Pyruvate kinase, alpha/beta domain |
| PMSR | PF01625.9 | −62 | Peptide methionine sulfoxide reductase |
| PP2C | PF00481.10 | −44 | Protein phosphatase 2C |
| PPDK_N | PF01326.8 | −87 | Pyruvate phosphate dikinase, |
| | | | PEP/pyruvate binding domain |
| PRA1 | PF03208.8 | 25 | PRA1 family protein |
| PSI_PsaF | PF02507.5 | 25 | Photosystem I reaction centre subunit III |
| PTR2 | PF00854.11 | −50 | POT family |
| PUA | PF01472.8 | 2.2 | PUA domain |
| Peptidase_A22B | PF04258.3 | −137.3 | Signal peptide peptidase |
| Peptidase_C1 | PF00112.11 | −115.8 | Papain family cysteine protease |
| Peptidase_C15 | PF01470.7 | −100 | Pyroglutamyl peptidase |
| Peptidase_M20 | PF01546.16 | −14.4 | Peptidase family M20/M25/M40 |
| Peptidase_S10 | PF00450.11 | −198 | Serine carboxypeptidase |
| Peptidase_S41 | PF03572.7 | −25.8 | Peptidase family S41 |
| PfkB | PF00294.12 | −67.8 | pfkB family carbohydrate kinase |
| Phytochrome | PF00360.9 | 11 | Phytochrome region |
| Pkinase | PF00069.14 | −70.8 | Protein kinase domain |
| Pkinase_C | PF00433.11 | 14 | Protein kinase C terminal domain |
| Pkinase_Tyr | PF07714.4 | 65 | Protein tyrosine kinase |
| Polysacc_synt_2 | PF02719.5 | −176 | Polysaccharide biosynthesis protein |
| Pro_CA | PF00484.8 | −45 | Carbonic anhydrase |
| Pro_dh | PF01619.7 | −120.5 | Proline dehydrogenase |
| Pyr_redox | PF00070.16 | 5 | Pyridine nucleotide-disulphide |
| | | | oxidoreductase |
| Pyr_redox_2 | PF07992.2 | −20 | Pyridine nucleotide-disulphide |
| | | | oxidoreductase |
| Pyr_redox_dim | PF02852.11 | −13 | Pyridine nucleotide-disulphide |
| | | | oxidoreductase, dimerisation domain |
| Pyridoxal_deC | PF00282.8 | −158.6 | Pyridoxal-dependent decarboxylase |
| | | | conserved domain |
| RHD3 | PF05879.2 | 25 | Root hair defective 3 GTP-binding protein |
| | | | (RHD3) |
| RIO1 | PF01163.11 | −89.1 | RIO1 family |
| RRM_1 | PF00076.10 | 15.2 | RNA recognition motif. (a.k.a. RRM, |
| | | | RBD, or RNP domain) |
| RTC | PF01137.11 | −36.9 | RNA 3′-terminal phosphate cyclase |
| RTC_insert | PF05189.3 | 25 | RNA 3′-terminal phosphate cyclase |
| | | | (RTC), insert domain |
| RWP-RK | PF02042.5 | 25 | RWP-RK domain |
| Ran_BP1 | PF00638.8 | −38 | RanBP1 domain |
| Ras | PF00071.11 | 18 | Ras family |
| Remorin_C | PF03763.3 | 25 | Remorin, C-terminal region |
| Response_reg | PF00072.11 | −14.4 | Response regulator receiver domain |
| Reticulon | PF02453.7 | −40 | Reticulon |
| Ribonuclease_T2 | PF00445.8 | −53 | Ribonuclease T2 family |
| Ribosomal_L1 | PF00687.10 | −101 | Ribosomal protein L1p/L10e family |
| Ribosomal_L10e | PF00826.7 | 25 | Ribosomal L10 |
| Ribosomal_L12 | PF00542.8 | 25 | Ribosomal protein L7/L12 C-terminal |
| | | | domain |
| Ribosomal_L19e | PF01280.9 | −28 | Ribosomal protein L19e |
| Ribosomal_L39 | PF00832.9 | 25 | Ribosomal L39 protein |
| Ribosomal_L7Ae | PF01248.13 | 6 | Ribosomal protein |
| | | | L7Ae/L30e/S12e/Gadd45 family |
| Ribosomal_S11 | PF00411.7 | −4 | Ribosomal protein S11 |
| Ribosomal_S17 | PF00366.9 | 1.7 | Ribosomal protein S17 |
| Ribosomal_S2 | PF00318.9 | −22 | Ribosomal protein S2 |
| Ribosomal_S27 | PF01599.8 | 50 | Ribosomal protein S27a |
| Rieske | PF00355.15 | −7 | Rieske [2Fe—2S] domain |
| RmlD_sub_bind | PF04321.6 | −171.8 | RmlD substrate binding domain |
| RuBisCO_small | PF00101.9 | −20.1 | Ribulose bisphosphate carboxylase, small |
| | | | chain |
| Rubrerythrin | PF02915.7 | −4.8 | Rubrerythrin |
| SAM_1 | PF00536.17 | 11.3 | SAM domain (Sterile alpha motif) |
| SAM_2 | PF07647.5 | 20 | SAM domain (Sterile alpha motif) |
| SPC25 | PF06703.1 | 25 | Microsomal signal peptidase 25 kDa |
| | | | subunit (SPC25) |
| SPX | PF03105.9 | −20 | SPX domain |
| SRF-TF | PF00319.8 | 11 | SRF-type transcription factor (DNA- |
| | | | binding and dimerisation domain) |
| START | PF01852.8 | 25 | START domain |
| SapB_1 | PF05184.4 | 20 | Saposin-like type B, region 1 |
| SapB_2 | PF03489.5 | 20 | Saposin-like type B, region 2 |
| SecY | PF00344.9 | −210 | eubacterial secY protein |
| SelR | PF01641.8 | −66.5 | SelR domain |
| Sigma70_r1_2 | PF00140.9 | 25 | Sigma-70 factor, region 1.2 |
| Sigma70_r2 | PF04542.3 | 11 | Sigma-70 region 2 |
| Sigma70_r3 | PF04539.4 | 10 | Sigma-70 region 3 |
| Sigma70_r4 | PF04545.5 | 20.7 | Sigma-70, region 4 |
| Sina | PF03145.6 | −48.4 | Seven in absentia protein family |
| Steroid_dh | PF02544.6 | −44.7 | 3-oxo-5-alpha-steroid 4-dehydrogenase |
| Suc_Fer-like | PF06999.2 | −42.4 | Sucrase/ferredoxin-like |
| Succ_DH_flav_C | PF02910.9 | −42 | Fumarate reductase/succinate |
| | | | dehydrogenase flavoprotein C-terminal |
| | | | domain |
| Sucrose_synth | PF00862.9 | −134 | Sucrose synthase |
| Sugar_tr | PF00083.12 | −85 | Sugar (and other) transporter |
| Synaptobrevin | PF00957.9 | 25 | Synaptobrevin |
| TPP_enzyme_C | PF02775.9 | 19.7 | Thiamine pyrophosphate enzyme, C- |
| | | | terminal TPP binding domain |
| TPP_enzyme_M | PF00205.11 | −23.9 | Thiamine pyrophosphate enzyme, central |
| | | | domain |
| TPP_enzyme_N | PF02776.7 | −70 | Thiamine pyrophosphate enzyme, N- |
| | | | terminal TPP binding domain |
| Thiolase_C | PF02803.6 | −30.7 | Thiolase, C-terminal domain |
| Thiolase_N | PF00108.11 | −129.5 | Thiolase, N-terminal domain |
| Thioredoxin | PF00085.8 | −25.7 | Thioredoxin |
| Tic22 | PF04278.2 | 25 | Tic22-like family |
| Transaldolase | PF00923.8 | −49 | Transaldolase |
| Transferase | PF02458.5 | −161.2 | Transferase family |
| Transket_pyr | PF02779.12 | −50 | Transketolase, pyridine binding domain |
| Transketolase_C | PF02780.9 | −15.5 | Transketolase, C-terminal domain |
| Transketolase_N | PF00456.10 | −98 | Transketolase, thiamine diphosphate |
| | | | binding domain |
| Trehalase | PF01204.8 | 25 | Trehalase |
| Trehalase_Ca-bi | PF07492.1 | 20 | Neutral trehalase Ca2+ binding domain |
| Trehalose_PPase | PF02358.6 | −49.4 | Trehalose-phosphatase |
| Trp_Tyr_perm | PF03222.3 | −232.6 | Tryptophan/tyrosine permease family |
| Trp_syntA | PF00290.10 | −149.8 | Tryptophan synthase alpha chain |
| Trypsin | PF00089.13 | −33.2 | Trypsin |
| Tub | PF01167.7 | −98 | Tub family |
| Tubulin | PF00091.14 | −55.7 | Tubulin/FtsZ family, GTPase domain |
| Tubulin_C | PF03953.6 | −10 | Tubulin/FtsZ family, C-terminal domain |
| UBA | PF00627.18 | 20.5 | UBA/TS-N domain |
| UDPGP | PF01704.7 | −265.2 | UTP--glucose-1-phosphate |
| | | | uridylyltransferase |
| UDPGT | PF00201.8 | −151 | UDP-glucoronosyl and UDP-glucosyl |
| | | | transferase |
| UPF0057 | PF01679.7 | 25 | Uncharacterized protein family UPF0057 |
| UbiA | PF01040.8 | −45 | UbiA prenyltransferase family |
| Ubie_methyltran | PF01209.8 | −117 | ubiE/COQ5 methyltransferase family |
| Usp | PF00582.15 | 25.7 | Universal stress protein family |
| VHS | PF00790.8 | −13.2 | VHS domain |
| VQ | PF05678.3 | 25 | VQ motif |
| W2 | PF02020.7 | 25 | eIF4-gamma/eIF5/eIF2-epsilon |
| WD40 | PF00400.19 | 21.4 | WD domain, G-beta repeat |
| WHEP-TRS | PF00458.9 | 10 | WHEP-TRS domain |
| WRKY | PF03106.5 | 25 | WRKY DNA-binding domain |
| Wzy_C | PF04932.4 | 25 | O-Antigen Polymerase |
| XET_C | PF06955.2 | 11.4 | Xyloglucan endo-transglycosylase (XET) |
| | | | C-terminus |
| Xan_ur_permease | PF00860.10 | −151.2 | Permease family |
| YL1 | PF05764.3 | 25 | YL1 nuclear protein |
| YL1_C | PF08265.1 | 18.6 | YL1 nuclear protein C-terminal domain |
| YTH | PF04146.5 | 25 | YT521-B-like family |
| Yippee | PF03226.4 | 25 | Yippee putative zinc-binding protein |
| YjeF_N | PF03853.3 | 25 | YjeF-related protein N-terminus |
| ZF-HD_dimer | PF04770.2 | 25 | ZF-HD protein dimerisation region |
| Zip | PF02535.10 | −28 | ZIP Zinc transporter |
| adh_short | PF00106.13 | −46.6 | short chain dehydrogenase |
| bZIP_1 | PF00170.10 | 16.5 | bZIP transcription factor |
| bZIP_2 | PF07716.4 | 15 | Basic region leucine zipper |
| cNMP_binding | PF00027.17 | 20.6 | Cyclic nucleotide-binding domain |
| cobW | PF02492.8 | −10 | CobW/HypB/UreG, nucleotide-binding |
| | | | domain |
| efhand | PF00036.19 | 17.5 | EF hand |
| ketoacyl-synt | PF00109.14 | −73.6 | Beta-ketoacyl synthase, N-terminal |
| | | | domain |
| malic | PF00390.8 | 25 | Malic enzyme, N-terminal domain |
| p450 | PF00067.11 | −105 | Cytochrome P450 |
| peroxidase | PF00141.12 | −10 | Peroxidase |
| tRNA-synt_2b | PF00587.14 | −40.5 | tRNA synthetase class II core domain (G, |
| | | | H, P, S and T) |
| ubiquitin | PF00240.12 | 19.4 | Ubiquitin family |
| zf-B_box | PF00643.13 | 11.1 | B-box zinc finger |
| zf-C2H2 | PF00096.14 | 19 | Zinc finger, C2H2 type |
| zf-C3HC4 | PF00097.12 | 16.9 | Zinc finger, C3HC4 type (RING finger) |
| zf-Dof | PF02701.5 | 25 | Dof domain, zinc finger |
| zf-LSD1 | PF06943.2 | 25 | LSD1 zinc finger |

### EXAMPLE 8

### Selection of Transgenic Plants with Enhanced Agronomic Trait(s)

This example illustrates the preparation and identification by selection of transgenic seeds and plants derived from transgenic plant cells of this invention where the plants and seed are identified by screening a having an enhanced agronomic trait imparted by expression of a protein selected from the group including the homologous proteins identified in Example 4. Transgenic plant cells of corn, soybean, cotton, canola, wheat and rice are transformed with recombinant DNA for expressing each of the homologs identified in Example 4. Plants are regenerated from the transformed plant cells and used to produce progeny plants and seed that are screened for enhanced water use efficiency, enhanced cold tolerance, increased yield, enhanced nitrogen use efficiency, enhanced seed protein and enhanced seed oil. Plants are identified exhibiting enhanced traits imparted by expression of the homologous proteins.

1-22. (canceled)

23. A plant cell with stably integrated, recombinant DNA comprising a promoter that is functional in plant cells and that is operably linked to DNA encoding a seven-in-absentia protein,
wherein said plant cell is present in a plant or seed that
(a) exhibits an enhanced trait as compared to control plants that do not have said recombinant DNA; and
(b) is derived from a progenitor plant or seed that was selected as having said enhanced trait from a population of plants or seeds that have said recombinant DNA, wherein said enhanced trait is selected from group consisting of enhanced water use efficiency, enhanced cold tolerance, increased yield, enhanced nitrogen use efficiency, enhanced seed protein and enhanced seed oil, and
wherein a portion of said population exhibits said enhanced trait.

24. The plant cell of claim 23 wherein said DNA encoding a seven-in-absentia protein is from a plant, bacteria or yeast and encodes a seven-in-abstentia protein having at least one domain of amino acids in a sequence that exceeds the Pfam gathering cutoff for amino acid sequence alignment with protein domain family identified by the Pfam name Sina; wherein the Pfam gathering cutoff for the Sina protein domain family is 48.4.

25. A plant cell of claim 23 wherein said protein has an amino acid sequence with at least 90% identity to an amino acid sequence in the group consisting of SEQ ID NO: 1138, 1139, 1321, 1429, 1430, 1431 and 1432.

26. A plant cell of claim 23 further comprising DNA expressing a protein that provides tolerance from exposure to an herbicide applied at levels that are lethal to a wild type of said plant cell.

27. A plant cell of claim 26 wherein the agent of said herbicide is a glyphosate, dicamba, or glufosinate compound.

28. A transgenic plant comprising a plurality of the plant cell of claim 23.

29. A transgenic seed comprising a plurality of the plant cell of claim 23.

30. A transgenic seed of claim 29 from a corn, soybean, cotton, canola, alfalfa, wheat or rice plant.

31. A transgenic pollen grain comprising a haploid derivative of a plant cell of claim 23.

32. A method for manufacturing non-natural, transgenic seed that can be used to produce a crop of transgenic plants with an enhanced trait resulting from expression of stably-integrated, recombinant DNA comprising a promoter that is (a) functional in plant cells and (b) is operably linked to DNA from a plant, bacteria or yeast that encodes a seven-in-absentia protein; wherein said enhanced trait is selected from the group of enhanced traits consisting of enhanced water use efficiency, enhanced cold tolerance, increased yield, enhanced nitrogen use efficiency, enhanced seed protein and enhanced seed oil; wherein said method comprises:
(a) screening a population of plants for said enhanced trait and said recombinant DNA, wherein individual plants in said population can exhibit said trait at a level less than, essentially the same as or greater than the level that said trait is exhibited in control plants which do not express the recombinant DNA,
(b) selecting from said population one or more plants that exhibit the trait at a level greater than the level that said trait is exhibited in control plants,
(c) verifying that said recombinant DNA is stably integrated in said selected plants,
(d) analyzing tissue of a selected plant to determine the production of a seven-in-absentia protein in plant cells of claim 23, and
(e) collecting seed from a selected plant.
